(19) United States
(12) Patent Application Publication
(10) Pub. No.: US 2014/0086836 A1
(43) Pub. Date: Mar. 27, 2014

(54) METHOD FOR DETECTION OF A NEUROLOGICAL DISEASE

(71) Applicants: Samantha C. Burnham, Doubleview (AU); Noel Faux, Parkville (AU); Simon M. Laws, Wanneroo (AU)

(72) Inventors: Samantha C. Burnham, Doubleview (AU); Noel Faux, Parkville (AU); Simon M. Laws, Wanneroo (AU)

(73) Assignees: MENTAL HEALTH RESEARCH INSTITUTE, Parkville, Victoria (AU); Commonweath Scientific and Industrial Research Organisation, Australian Capital Territory (AU); NATIONAL AGEING RESEARCH INSTITUTE, Parkville, Victoria (AU); EDITH COWAN UNIVERSITY, Joondalup (AU)

(21) Appl. No.: 14/116,114

(22) PCT Filed: May 3, 2012
(86) PCT No.: PCT/AU2012/000475

§ 371 Date: Nov. 26, 2013

(30) Foreign Application Priority Data
May 3, 2011 (AU) 2011901634

(51) Int. Cl. G01N 33/68; G06F 19/20

(52) U.S. Cl. CPC G01N 33/6896; G06F 19/20
USPC 424/1.81; 506/9; 506/18; 506/16; 702/19

(57) ABSTRACT

The present invention provides methods for predicting whether a subject will develop a disease capable of affecting cognitive function. More specifically, the present invention relates to the predictive detection of neurological diseases in a subject. The methods and systems provided enable a quantitative assessment and theoretical predictions of neocortical amyloid loading or amyloid beta levels based on the measurement of biomarkers in biological fluids that will provide an indication of whether a subject is likely to develop a neurological disease, such as Alzheimer's disease (AD).

### FIELD OF THE INVENTION

The present invention relates to methods for predicting the prognosis of a neurological disease that affects cognitive function. More specifically, the present invention relates to the prognostic detection of neurological diseases, associated with elevated amyloid load, such as Alzheimer's disease, in a subject.

### BACKGROUND OF THE INVENTION

Alzheimer's disease (AD) is a neurodegenerative disease characterised by memory loss and results in a progressive loss of cognitive function and dementia affecting one in eight people by the time they reach 65 years of age. Neuropathologically, AD is characterised by the presence of neuritic plaques (NP), neurofibrillary tangles (NFT), and neuronal loss, along with a variety of other findings.

AD can strike persons as young as 40-50 years of age, however because the presence of the disease is difficult to determine without invasive techniques such as brain biopsies, the time of onset is generally unknown. In practice, AD is definitively diagnosed through examination of brain tissue, however other semi-diagnostic techniques are often utilised, such as clinical criteria evaluation of the subject in question. Due however to the invasive and dangerous nature of brain biopsies and the limited confidence that clinical criteria evaluation provides, the only guaranteed manner for conclusive diagnosis of AD is usually that provided under autopsy. Post-mortem slices of brain tissue of victims of AD exhibit the presence of amyloid in the form of proteinaceous extracellular cores of the neuritic plaques that are characteristic of AD.

The diagnosis (or prognosis) of AD prior to any cognitive decline represents a limiting step where considerable time, expertise and intellectual effort have been (and further need to be) expended. This assumes that the AD etiology starts prior to any cognitive decline and thus introduces the concept of preclinical AD. Support for the preclinical AD concept is given by subtle cognitive deficits present decades prior to dementia diagnosis and post-mortem observations showing that the neuropathology of AD, including cerebral deposits of amyloid plaque, is present in more than a quarter of non-demented persons aged over 75 years (which equates to the prevalence of dementia at age 85 years).

The ability to detect preclinical or early stage disease through reliable measurement of markers present in biological samples from a subject suspected of having AD would also allow treatment and management of the disease to begin earlier. Accordingly, research efforts have focused on developing non-invasive methods for diagnosing AD in vivo that include imaging techniques, or biochemical or genomic methods for detection of biomarkers.

Imaging techniques have been available for many years. For imaging of diseases of the brain such as amyloid fibril or plaque forming neurological diseases, a series of uncharged derivatives of thioflavin T have been developed as amyloid-imaging agents and radiotracers that exhibit high affinity for amyloid deposits and high permeability across the blood-brain barrier. Extensive in vitro and in vivo studies of these amyloid-imaging agents represented by the thioflavin BTA-1 suggest that they specifically bind to amyloid deposits at concentrations typical of those detectable during positron emission tomography studies. In the complex milieu of human brain, non-specific binding of the amyloid-imaging compounds is low, even in control brains devoid of amyloid deposits.

The best validated of these amyloid-imaging agent is Pittsburgh Compound-B (PiB; Klunk et al, Annals of Neurology, 55, 306-319, 2004), which is an analogue of the amyloid-binding dye Thioflavin-T. PiB-Positron Emission Tomography (PiB-PET) studies in AD have shown robust cortical binding of PiB with amyloid plaque (Klunk et al., 2004; Kemppainen et al, Neurology, 67, 1575-1580, 2006, Rowe et al, Neuropsychologia, 46, 1688-1697, 2008, Ng et al Journal of Nuclear Medicine, 48, 547-552, 2007). This provides a promising early and accurate detection marker, perhaps what could be considered the gold standard. In vitro PiB binds specifically to extracellular and intravascular fibrillar Aβ deposits in post-mortem AD brains PiB does bind non-specifically to white matter, likely due to delayed clearance of the lipophilic compound from white matter (Fodero-Tavoletti M. T., et al., J Nucl Med; 50(2):198-204 2009). Recently other compounds have been investigated based on the similar functionality of PiB to target Amyloid beta, such as AV-45 (florpiramine F-18)—otherwise known as F-18 AV-45) produced by Avid Radiopharmaceuticals Pty Ltd (Philadelphia).

PET imaging however is limited in the detection of AD in a vast majority of the population due to high costs, limited availability of the instrumentation and by the short half-lives of the radiotracers currently in use. Thus, as early screening tools for AD, imaging agents and measurements are impractical and a simple blood test for early diagnosis or, better, prognosis is desirable.

Identification of biomarkers for early detection of brain diseases has become a more recent research focus. Biomarkers in cerebral spinal fluid (CSF) have been found to be a powerful confirmatory assessment of some neurological diseases for which diagnosis by imaging has been performed (Shaw et al. Nature Reviews Drug Discovery, 6, 295-303, 2007, Hampel et al. Alzheimers & Dementia, 4, 38-48, 2008, Hampel et al. Nature Reviews Drug Discovery, 9, 560-574, 2010, Dubois et al. Lancet Neurology, 9, 1118-1127, 2010). However this requires an invasive lumbar puncture to sample the CSF. Furthermore, studies using specific cerebrospinal fluid markers (for example, increased phosphophorylated tau and decreased amyloid-beta levels) have yet to show commercial and medical value as markers of disease state.

Endeavours to obtain a simple blood test for AD have had little success to date and early diagnosis prior to the onset of any clinical symptoms remains particularly challenging. However a number of recent studies (Ray et al, Nature Medicine, 13, 1359-1362, 2007 and O'Bryant et al, Alzheimer's Res 2010) have proposed panels of biomarkers with diagnostic powers for AD. However these recent tests have found not to be reproducible and there has been criticism about the small cohort (sample size) used to generate the results.

Some commercial kits with specific biomarker panels are now available whose choice is based on studies out of Kings College London (Proteomics Sciences, Thambisetty et al, PLoS ONE, 6, 2001). These kits however use expensive instrumentation such as mass spectrometry and in some cases require use of CSF.

Accordingly, more readily available screening methodologies that can also provide early detection for AD that would allow simple, cheap and effective screening to be performed and in turn could provide the justification for confirmatory CSF or confirmatory brain imaging tests.

A need therefore exists to provide an improved system capable of early and economic prognosis of Alzheimer's or Alzheimer-like diseases which can provide assistance to clinicians in reaching an early stage prognosis prior to the portrayal of detectable clinical indicators and which would obviate the need for actual confirmatory brain imaging tests.

With disease modifying therapies for AD undergoing clinical trials, there is a social and economic imperative to identify biomarkers that can detect features of the disease in at-risk individuals in the earliest possible stage, so anti-AD therapy can be administered at a time when the disease burden is mild and it may prevent or delay functional and irreversible cognitive loss.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

### SUMMARY OF THE INVENTION

There is a need for an improved method of identifying subjects with a neurological disease such as AD, particularly at the onset of the disease and before clinical symptoms arise. The early identification of AD would thus assist in delaying disease progression through early intervention. To this end, the present inventors have developed methods and systems to provide a quantitative assessment of neocortical amyloid loading or amyloid beta levels based on biological fluid measurements. Accordingly, they provide a marker based detection system derived from biological fluid and optionally clinical measurements that correlates with neocortical amyloid loading, as imaged by radiological assessment, such as PiB-PET. Given the proven association of neocortical amyloid load with AD and the desirability of blood based tests, the present invention holds promise as an early screening tool for the population to differentiate those at risk of developing AD.

Through adoption of the methods and systems described, the present inventors are capable of identifying a collection of biomarkers, present in a biological sample of an individual (e.g. blood, including serum or plasma), whose combined levels are altered in individuals with a neurological disease or at risk of developing a neurological disease, such as Alzheimer's disease (AD). The inventions have also identified biomarkers altered in individuals with a neurological disease that can be utilised to provide a prognostic indication of the likelihood of an individual having or potentially developing AD.

It is considered in the methods of the present invention that biomarkers capable of predicting the levels of neocortical amyloid loading can be determined by the comparison of a plurality of predetermined validated samples of subjects that have been assayed for a series of biological markers and whose level of neocortical amyloid loading has been ascertained. The comparison is carried out using a set of relevant coefficients that have been derived from the plurality of predetermined validated samples of subjects. It is further considered that the plurality of predetermined validated samples of subjects can be obtained from cohort data sets or cohort studies. Whilst it is possible that multiple cohort data sets or results from cohort studies can be utilised in the methods of the present invention to determine the relevant coefficients and/or biological markers to predict levels of neocortical amyloid loading, it is envisioned that only one cohort data set may be needed, provided that the cohort data set comprises values for both biological markers and evaluated values for the level of neocortical amyloid loading from the same assayed individuals.

In an aspect of the present invention, there is provided a method for the prognosis, or for the aiding in the diagnosis, of a neurological disease in an individual by measuring the amount of one or more biomarkers in a biological sample, such as a biological sample from the individual, and correlating a signature of the biomarkers measured and calculating a predictive AD status based on the theoretical level of neocortical amyloid loading. According to the present invention, there are provided methods for the generation of a set of relevant coefficients based on biomarkers and optionally clinical markers, either combined or separate, which are capable of being used to predict neocortical amyloid loading, and thus provide an indication of the likelihood of a subject developing AD by determining from a biological sample from a subject, their theoretical neocortical amyloid beta level.

In a further aspect of the present invention, there is provided a method for detecting biomarkers that qualify the AD status of a subject, said biomarker(s) being detectable in a biological sample where the biological fluid would be suspected to also comprise increased levels of amyloid precursor protein or amyloid beta peptide.

Also provided are peptides, polypeptides, proteins, oligonucleotides, fragments thereof, and/or other markers, such as metals, metabolites or vitamins, identifiable in the biological fluid and kits comprising the peptides, polypeptides, proteins, oligonucleotides, fragments thereof, and/or other markers such as metals, metabolites or vitamins identifiable in the biological fluid that may be used to determine the identity of the biomarkers that are likely to indicate that a subject possesses AD or is likely to develop AD.

Other aspects of the present invention will become apparent to those ordinarily skilled in the art upon review of the following description of specific embodiments of the invention.

### BRIEF DESCRIPTION OF THE FIGURES

For a further understanding of the aspects and advantages of the present invention, reference should be made to the following detailed description, taken in conjunction with the accompanying drawings.

FIG. 1 (a) shows Receiver Operating Characteristics (ROC) for cross-validated predictions of the neocortical amyloid load of 273 subjects from the Australian Imaging, Biomarkers and Lifestyle (AIBL) study.

FIG. 1 (b) shows the correlation of the predicted Baseline Neocortical amyloid load with the actual measured neocortical amyloid load (as given by the PiB-PET Standardized Update Value ratio (SUVR)) for 273 subjects from the Australian Imaging, Biomarkers and Lifestyle (AIBL) study.

FIG. 2 (a) shows the actual measured neocortical amyloid load (as given by the PiB-PET Standardized Update Value ratio (SUVR)) for 273 subjects from the Australian Imaging, Biomarkers and Lifestyle (AIBL) study, grouped by clinical diagnosis.

FIG. 2 (b) shows the predicted neocortical amyloid load (as given by the PiB-PET Standardized Update Value ratio (SUVR)) for 817 non-imaged subjects from the Australian Imaging, Biomarkers and Lifestyle (AIBL) study, grouped by clinical diagnosis.

FIG. 3 shows the percentage of participants predicted to have high neocortical amyloid loading, with respect to their clinical diagnosis at baseline and 18 month transitions.

FIG. 4 shows Receiver Operating Characteristics (ROC) for predictions of the neocortical amyloid load of 74 subjects from the Alzheimer's Disease Neuroimaging Initiative (ADNI) study.

FIG. 5 shows a statistical analysis flow chart of methods utilised for determining and generating an appropriate model.

FIG. 6 shows a venn diagram of blood markers chosen by the different multivariate models along with their associated sensitivities and specificities; B: ROC curves for the cross-validated Random Forest models applied to the imaged AIBL sub-cohort; C: ROC curves for Random Forest models applied to the imaged ADNI sub-cohort. [Orange=M1, Blue=M2, Pink=M3, Grey=M4].

FIG. 7 (a) shows Actual SUVR values for the imaged AIBL sub-cohort split by Clinical Diagnosis.

FIG. 7 (b) shows Predicted SUVR values for the non-imaged AIBL sub-cohort split by Clinical Diagnosis.

### DETAILED DESCRIPTION OF THE INVENTION

### 1. Biomarkers

The present invention provides for the selection, identification, or use of a plurality of biomarkers to predict a theoretical value for amyloid load which in turn helps identify the risk to a subject of developing amyloid plaque forming related disease such as AD or ascertaining the progressing of AD or AD like disease in a subject.

Particular biomarkers suitable for use in this invention include those already identified in the prior art as having diagnostic power. While individual biomarkers are useful diagnostic biomarkers, it has been found that a combination of biomarkers can provide greater predictive value of a particular status than single biomarkers alone. Specifically, the detection of a plurality of biomarkers (often referred to as a “biomarker profile” or a “biomarker fingerprint”) in a sample, or the use of a plurality of biomarkers as a panel for use in the analysis of a sample, can increase the sensitivity and/or specificity of the test. Biomarkers have typically been found in either blood, plasma, serum or CSF, urine or other fluids and these would all be possible to choose from in this invention.

Some specific protein based biomarkers include age and ApoE genotype (Neurology 1997; 48:139-147); vitronectin (VTN or protein S) and protectin (CD59) which are all believed to inhibit membrane insertion (Brain Res. 1992 May 8; 579(2):337-4); Immunoglobulin Growth Factor binding protein 2 (IGF-BP2) which has been shown to be higher in the CSF of individuals with AD. IGF-BP2 has also been correlated with the levels of CSF tau. (Biofactors 2008; 33:99-106, Journal of Neural Transmission-Parkinson's Disease and Dementia Section 1993; 5:165-176, Plos One 2011; 6). Proteins such as antithrombin 3, (AT3) anti-chymotrypsin (ACT), and zinc α2 glycoprotein (ZAG) are abundant proteins found in CSF and were found to give comparable predictive powers to Amyloidβ42 and Tau protein (Proteomics Clan. Appl. 2007, 1). Further non-blood based biomarkers that may be suitable for AD diagnosis include the markers discovered in CSF, such as osteopontin, ubiquitin, C4A des-Arg, α2 microglobulin (Arch Neurol, 64 (3) 2007, 366-37). Other proteins showing some differential change between AD and control subjects have also included transthyretin S-glutathionylated, Cystatin C, ubiquitin, vasostatin II fragment, pancreatic ribonuclease, osteopontin prostaglandin-D synthase, chromogranin B peptide and transferrin

Panels with immune response and or immune signalling proteins such as chemokine (C-X-C motif) ligand BLC (CXCL13), IgM, IL-17, VCAM1, CD40, C-reactive-protein (CRP) and again IGF-BP2, have also been described and could be used in this invention (PCT/AU2010/001575). U.S. Pat. No. 7,993,868 describes identification of three markers from CSF using mass spectrometry Saposin D, FAM3C and beta-2-microglobulin that had some correlation with AD status. WO2011/143574 describes a preferred panel derived from the University of Penn study which was made up of cortisol, pancreatic polypeptide, osteopontin, IGF BP2, and resistin. There were other biomarkers also identified in this study, but were not considered as robust as these five. These other markers included the alpha-1 macroglobulin, angiopoietin-2, Apoliprotein E, beta-2 microglobulin, BLC, E-selectin, FAS, fatty acid binding protein, interleukin 10, PAPP-A, stem cell factor, thrombinmodulin, cortisol, hepatocyte Growth factor, NT-Pro-BNP, TIMP-1, VCAM-1, VEGF, and Von Willebrand factor

A multitude of further potential protein biomarkers have also been described in WO2011/142901, where the preferred ones were hematopoietic SH2 domain containing protein (HSH2D), pentatricopeptide repeat containing protein 2 (PTCD2), 60S ribosomal protein L41 (RPL41) or FERM domain-containing protein 8 (FRMD8). A list of potential biomarkers derived from multiple clinical cohort results is described in US200510221348 with the preferred list of 7 being brain derived neurotropic factor (BDNF), soluble interleukin-6 receptor (sIL-6R), II-8, MIP-1 gamma, platelet derived growth factor BB homodimeric (PDGF-BB), and tissue inhibitor of metalloproteases-1 (TIMP-1).

In addition, a multitude of proteins or peptides may also be of aid in neurological disease detection as described in US2011/0129920, which relates to the identified by mass spectrometry of whole proteins or fragments of Hemopexin, Ubiquitin-3aa, Pancreatic ribonuclease, Transthyretin, Cystatin, Secretoneurin, Vasostatin II, Chromogranin A-beta 1-40, Chromogranin B Apolipoprotein A-II dimer, C3a des-Arg, Prostaglandin-D synthase, Alpha-1-antichyrnotrypsin, Osteopontin, VGF, Thymosin, Albumin, Beta-2-Microglobulin, transferrin, to name a few.

It is considered that any of the above referenced markers or biomarkers, and any other biomarker from other varied sample sources, could be utilised according to the methods of the present invention in the identification of a biomarkers that are present in subjects to qualify the neurological disease status in the subject.

The present invention provides for the identification of biomarkers that are present in subjects having a neurological disease, such as AD, versus subjects that are absent or not fully indicative of the disease, and provides methods utilising the identified biomarkers to qualify the neurological disease status in a subject. The present invention also provides methods for identifying therapeutics relevant to neurological diseases, such as AD, and for monitoring progression of neurological diseases. It further provides a method for prediction of a theoretical score of neocortical amyloid loading from the presence of the identified characteristic biomarkers that can enable an improved prediction of the likelihood of an individual developing AD or an AD like disease.

The present invention generally encompasses the identification of biomarkers, identification of the presence of biomarkers in an individual, methods of using the identified biomarkers to assess the risk or the status of a subject having a neurodegenerative disease, such as AD.

Accordingly, an aspect of the present invention includes a method for generating a set of relevant coefficients for predicting neocortical amyloid beta levels, comprising a) applying a classification algorithm to a plurality of biomarker values from a plurality of predetermined validated samples; and b) applying the classification algorithm to a plurality of amyloid beta levels obtained from the same plurality of predetermined validated samples of step a); wherein applying the classification algorithm generates a set of relevant coefficients capable of predicting the amyloid beta levels by correlating biomarkers to amyloid beta levels.

For the purpose of brevity, some of the following description will be made in the context of AD. It is considered however that the skilled addressee would be capable of understanding that the present invention may also be used as a prognostic or in aiding in the diagnosis and/or monitoring of the progression of other neurological diseases associated with amyloid plaque build-up. Further, it is considered that the skilled addressee would understand that the present invention may also be useful for stratifying patients according to the severity of other neurological diseases, such as those associated with neural degeneration and amyloid plaque build-up, including, but not limited to, Parkinson's disease (PD) and dementia associated with Lewy bodies, amyloid plaque forming diseases and AD like diseases.

In the present invention, the inventors propose that the presence of a plurality of biomarkers, two of which are Amyloidβ42 and ApolipoproteinE (Genotype), can be used to generate a signature that can predict amyloid loading which then assists in determining whether a subject will be likely to develop a neurological disease associated with an increased level or build-up of amyloid plaque, and that these may therefore provide appropriate targets for use in methods of assessment of whether an subject possesses or is likely to develop a neurological disease such as AD.

The inventors further propose that the presence of a plurality of biomarkers for ascertaining whether a subject will be likely to develop a neurological disease associated with an increased level or build-up of amyloid plaque can also be provided by the selected use of other biomarkers, comprising any of those selected from group list including 6Ckine, ABeta 42 (AB42), diponectin, Agouti-Related Protein, Aldose Reductase, Alpha.2.Macroglobulin, Alpha-1-Antichymotrypsin, Alpha-1-Antitrypsin—A1AT, Alpha-1-Microglobulin, Alpha-2-Macroglobulin, alpha1 acid glycoprotein, alanine transaminase—ALT, albumin—Alb, alkaline phosphatase—AP, alpha syncline, Alpha-Fetoprotein—AFP, Amphiregulin, Angiogenin, Antithrombin 3—AT3, Angiopoietin-2—ANGPT.2, Angiotensin-Converting Enzyme—ACE, CD143, Angiotensinogen, Annexin A1, ApoE_ECU, Apolipoprotein AII Dimer, Apolipoprotein B—Apo.B. Apolipoprotein C-I, Apolipoprotein, D—Apo.D, Apolipoprotein E—Apo.E, Apolipoprotein H—Apo.H, Apolipoprotein(a), Apolipoprotein.CIII, Ast, AXL Receptor Tyrosine Kinase—AXL, B cell-activating factor, B Lymphocyte Chemo attractant—BLC, B12, Baso, Bcl-2-like protein 2, Beta-2-Microglobulin—B2M, Betacellulin, Beta 2 microglobulin, Bilirubin, Bone Morphogenetic Protein 6—BMP.6, Brain-Derived Neurotrophic Factor—BDNF, C3, Caer, Calbindin, Calcitonin, Cancer Antigen 125, Cancer Antigen 15-3, Cancer Antigen 19-9, Cancer Antigen 72-4, Carcinoembryonic Antigen—CEA, Cathepsin D, CD 40 antigen—CD40, CD40.Ligand, CD5 Antigen-like, ceurolplasmin, CgA, chemokine (C-X-C motif), Chemokine CC-4—CK.MB, Chromogranin-4, Ciliary Neurotrophic Factor, CI, Clusterin, Collagen IV, Complement C3, Complement.Factor.H, Connective Tissue Growth Factor, Cortisol, C-Peptide, C-Reactive Protein—CRP, Creatine Kinase-MB, chromogranin B, Endoglin, Endostatin, Endothelin-1, Eos, Eotaxin (all subunits), Epidermal Growth Factor—EGF, Epidermal Growth Factor Receptor—EGF.R, Epiregulin, Epithelial cell adhesion molecule, Erythropoietin, E-Selectin, extracellular newly identified RAGE-binding protein—EN.RAGE, Ezrin, erythrocyte sedimentation rate—ESR, estimated glomerular filtration rate—eGFR, Factor.VII, FAS, FASLG Receptor, Family with sequence similarity 3, member C (FAM3C(I)), Fatty Acid-Binding Protein, Ferritin, Fetuin-A, Fibrinogen, Fibroblast Growth Factor 4, Fibroblast Growth Factor basic, Fibulin-1C, Follicle-Stimulating Hormone—FSH, FT3, G, Galectin-3, Gelsolin, gamma glutamyl transpeptidase—GGT, Glucagon, Glucagon-like Peptide 1, total—GLP.1.total, Glucose-6-phosphate Isomerase, Glutamate-Cysteine Ligase Regulatory subunit, Glutathione S-Transferase alpha, Glutathione S-Transferase Mu 1, Granulocyte Colony-Stimulating Factor—G.CSF, GRO.alpha, Growth Hormone—GH, Haptoglobin, HCC.4, HCY, HE4, Heat Shock Protein 60, Heparin-Binding EGF-Like Growth Factor—HB.EGF, Hepatocyte Growth Factor—HGF, Hepatocyte Growth Factor receptor, Hepsin, hemopexin, Human Chorionic Gonadotropin beta, Human Epidermal Growth Factor Receptor 2, Hemoglobin—Hb, high density lipoprotein—HDL, iron—Fe, Immunoglobulin A—IgA, Immunoglobulin E—IgE, Immunoglobulin M—IgM, Inno_AB_ratio, Inno_AB40, Inno_AB42, Insulin, Insulin-like Growth Factor Binding Protein 4, Insulin-like Growth Factor Binding Protein 5, Insulin-like Growth Factor Binding Protein 6, Insulin-like Growth Factor-Binding Protein 1, Insulin-like Growth Factor-Binding Protein 2, Insulin-like Growth Factor-Binding Protein 2—IGF.BP.2, Insulin-like Growth Factor-Binding Protein 3, Intercellular Adhesion Molecule 1—ICAM.1, Interferon gamma, Interferon gamma Induced Protein 10, Interferon-inducible T-cell alpha chemo attractant, Interleukin-1 alpha, interleukin-1 beta, Interleukin-1 receptor antagonist, Interleukin-1 receptor antagonist—IL.1ra, Interleukin-10, Interleukin-10—IL.10, Interleukin-12 Subunit p40, Interleukin-12 Subunit p70, Interleukin-12 Subunit p70—IL.12p70, Interleukin-13—IL.13, Interleukin-15—IL.15, Interleukin-16—IL.16, Interleukin-17—IL.17, Interleukin-18—IL.18, Interleukin-2, Interleukin-2 receptor alpha, Interleukin-2, Interleukin-3—IL.3, Interleukin-4 IL.4, Interleukin-5—IL.5, Interleukin-6 receptor, Interleukin-7, Interleukin-8—IL.8, Kallikrein 5, Kallikrein-7, Kidney Injury Molecule-1, Lactoylglutathione lyase, Latency-Associated Peptide of Transforming Growth Factor beta 1, Lectin-Like Oxidized LDL Receptor 1, Leptin, Lipoprotein.a, Luteinizing Hormone—LH, long-chain plasma ceramides C22:0, long-chain plasma ceramides C24:0 Lymphotactine—Lymp, low density lipoprotein—LDL, Macrophage Colony-Stimulating Factor 1—M.CSF, Macrophage inflammatory protein 3 beta, Macrophage Inflammatory Protein-1 alpha, Macrophage Inflammatory Protein-1 beta, Macrophage Inflammatory Protein-3 alpha, Macrophage Migration Inhibitory Factor, Macrophage-Derived Chemokine—MDC, Macrophage-Stimulating Protein, Malondialdehyde—Modified Low-Density Lipoprotein, Maspin, Matrix Metalloproteinase-1, Matrix Metalloproteinase-10, Matrix Metalloproteinase-2-MMP.2, Matrix Metalloproteinase-3, Matrix Metalloproteinase-7, Matrix Metalloproteinase-9—MMP.9, mean corpuscular hemoglobin, concentration—MCHC, mean platelet volume—MPV, melanin concentrating hormone—MCH, membrane cofactor protein 1—MCP.1, modified citrullinated vimentin—MCV, Mehta_AB_ratio, Mehta_AB40, Mehta_AB42, Mesothelin, Metals Mean Chromium.isotope.52, Metals Mean Chromium isotope.53, Metals Mean Copper isotope.65, Metals Mean Iron isotope.57, Metals Mean Rubidium.isotope.85, —Metals Mean Selenium isotope.78, Metals Mean Zinc isotope 66, MHC class I chain-related protein A, Microalbumin, MIP.1 beta, MIP.1alpha, Mono, Monocyte Chemotactic Protein 1, Monocyte Chemotactic Protein 2, Monocyte Chemotactic Protein 3, Monocyte Chemotactic Protein 4, Monokine Induced by Gamma Interferon, MPO, Myoglobin, Myeloid Progenitor Inhibitory Factor 1—MIF, Myeloperoxidase, neutrophil-activating peptide—ENA.78, Nerve Growth Factor beta, Neuronal Cell Adhesion Molecule—NrCAM, Neuron-Specific Enolase, Neuropilin—1, Neutrophil Gelatinase-Associated Lipocalin, Neutrophils, N-terminal prohormone of brain natriuretic peptide, Nucleoside diphosphate kinase B, Oestradiol, Osteopontin, Osteoprotegerin, Parkinson protein 5, Parkinson protein 7, Platelet count—Plt, potassium—K, PPY—Pancreatic.polypeptide, Packed cell volume—PCV, PAI.1, Pancreatic ribonuclease—PARC, Pepsinogen I, Peptide YY, Peroxiredoxin-4, Phosphoserine Aminotransferase, Placenta Growth Factor, Plasminogen Activator Inhibitor 1, Platelet-Derived Growth Factor BB—PDGF, Pregnancy-Associated Plasma Protein A—PAPP.A, PRL, Progesterone, Proinsulin-intact, Proinsulin-Total, Prolactin, Prostasin, Protein I-309, Prostate-Specific Antigen, Free-.PSA.Free, prostaglandin D synthase, Prostatic Acid Phosphatase—PAP, Protein S100-A4, Protein S100-A6, Pulmonary and Activation-Regulated Chemokine, Receptor tyrosine-protein kinase erbB-3, Resistin, red blood cell distribution width—RDW, Red Cell count—RCC, red cell folate—rFol, saposin A, saposin B, saposin D, saturated transferrin—tr.sat, serum folate—sFol, sodium—Na, stem cell factor—SCF, S100 calcium-binding protein B, Secretin, Serotransferrin, secretoneurin, Serum Amyloid P-Component—SAP, Serum Glutamic Oxaloacetic Transaminase—SGOT, Sex Hormone-Binding Globulin—SHBG, Sortilin, Squamous Cell Carcinoma Antigen-1, sRAGE, Stromal cell-derived factor-1, Superoxide Dismutase 1, soluble—SOD, T Lymphocyte-Secreted, Tamm-Horsfall Urinary Glycoprotein, T-Cell, Specific Protein RANTES—RANTES, TECK, Tenascin-C, Testosterone-Total, Tetranectin, Thrombomodulin, Thrombopoietin, thymosin beta, Thrombospondin-1, Thyroglobulin, Thyroid-Stimulating Hormone—TSH, Thyroxine-Binding Globulin—TBG, Tissue Factor, Tissue Inhibitor of Metalloproteinases 1—TIMP.1, Tissue type Plasminogen activator, testosterone-Testo, Thrombospondin 1—THBS1, Thyroxine, FT4, total calcium—Ca, total protein—tPr, TNC, TNF-Related Apoptosis inducing Ligand Receptor 3, TRAIL.R3, transferrin, Transforming Growth Factor alpha, Transforming Growth Factor beta-3, Transthyretin, Trefoil Factor 3, Trig, Tumor Necrosis Factor alpha, Tumor Necrosis Factor beta, Tumor necrosis factor receptor 2—TNF.RII, Tumor Necrosis Factor Receptor 1, Tyrosine kinase with Ig and EGF homology domains 2, Urea, Urokinase-type Plasminogen Activator, Urokinase-type plasminogen activator receptor, ubiquitin 3, ubiquitin 4, Vascular Cell Adhesion Molecule-1, Vascular, Endothelial Growth Factor, Vascular endothelial growth factor B, Vascular Endothelial Growth Factor C, Vascular endothelial growth factor D, Vascular Endothelial Growth Factor Receptor 1, Vascular, Endothelial Growth Factor Receptor 2, Vascular endothelial growth factor receptor 3, Vitamin K-Dependent Protein S, Vitronectin, YKL-40, Vascular cell adhesion protein 1—VCAM.1, Vascular endothelial growth factor—VEGF, von Willebrand factor—vWF, or white cell count—WCC

The inventors further propose that the presence of a plurality of biomarkers for the ascertaining whether a subject will be likely to develop a neurological disease associated with an increased level or build-up of amyloid plaque can also be provided by the selected use of other biomarkers, comprising any of those chosen from the list provided in TABLE 8.

In a further embodiment, the set of biomarkers selected are Amyloidβ42 and Apolipoprotein E when combined with at least one further marker as provided in TABLE 8 and when combined with at least one clinical markers selected from the list comprising Gender, Location Sampled, Community Involvement, Anaemia Status, Age, Marital Status, Years of Education, Physical Activity Quartile, Intra Cranial Volume, Hippocampal Volume, Clinical Dementia Rating (CDR) sum of boxes, or Body Mass Index and which can be also be used to generate a signature that can predict amyloid loading which then assists in determining whether a subject will be likely to develop a neurological disease, and that these may therefore provide appropriate targets for use in methods of assessment of whether an subject possesses or is likely to develop a neurological disease such as AD.

In a preferred embodiment the set of biomarkers selected are Amyloidβ42 and Apolipoprotein E when combined with at least one further marker selected from the list comprising cortisol, IgM, IL-17, PPY, VCAM1, or BLC. Alternatively, the selected set of biomarkers can comprise Amyloidβ42 and Apolipoprotein E when combined with at least one clinical marker, such as at least one of Gender, Location Sampled, Community Involvement, Anaemia Status, Age, Marital Status, Years of Education, Physical Activity Quartile, Intra Cranial Volume, Hippocampal Volume, Clinical Dementia Rating (CDR) sum of boxes, or Body Mass Index which can be also be used to generate a signature that can predict amyloid loading which then assists in determining whether a subject will be likely to develop a neurological disease, and that these may therefore provide appropriate targets for use in methods of assessment of whether an subject possesses or is likely to develop a neurological disease such as AD.

### 2. Definitions

The terms “Alzheimer's patient”, “AD patient”, and “individual diagnosed with AD” as used herein, are considered to all refer to an individual who has been diagnosed with AD or has been given a probable diagnosis of Alzheimer's Disease (AD).

The term “biomarker” as used herein, includes, but is not considered limited to, proteins, polypeptides, polynucleotides and/or metabolites present in a biological sample (e.g., metals, or vitamins and the like) whose value (e.g. concentration, expression and/or activity) in a biological sample from a subject or a control population. It is further considered that a biomarker can also comprise a fragment, or portion, or derivate thereof of a protein, polypeptides, polynucleotides and/or metabolites of interest. Any listed biomarkers are considered to also include their gene and protein synonyms. A biomarker is an organic biomolecule that is present in a sample taken from a subject of one phenotypic status (e.g., having a disease). A biomarker, alone or in combination is considered statistically relevant if the value of it or its relationship with other biomarkers is different to other phenotypic statuses. Common tests for statistical significance include, among others, t-test, X2 test, ANOVA, Kruskal-Wallis, Wilcoxon, Mann-Whitney and odds ratio. Biomarkers, alone or in combination, provide measures of relative risk that a subject belongs to one phenotypic status or another. Therefore, biomarkers are conventionally useful as markers for indicating the likelihood that a subject will develop a disease (prognostic), possess a disease (diagnostic) or ascertain the therapeutic effectiveness of a drug (theranostics) and drug toxicity,

The term “AD biomarker” and the like as used herein, is not intended to indicate the biomarker is only to be used in the prognosis, aid in the diagnosis, monitoring or stratifying of an individual with AD. As this disclosure makes clear, the biomarkers of the present invention are also useful for, for example, assessing cognitive function, assessing MCI, stratifying AD, etc., as well as assessing cognitive function and stratifying other neurological diseases, such as those associated with neurodegeneration.

The term “AD biomarker polynucleotide” as used herein, refers to any of: a polynucleotide sequence encoding an AD biomarker, the associated trans-acting control elements (e.g., promoter, enhancer, and other gene regulatory sequences), and/or mRNA encoding the AD biomarker.

Reference to “AD prognosis markers”, “AD diagnosis markers”, “AD biomarker” and “biomarker” (used interchangeably herein) are terms of convenience to refer to the markers described herein and their use, and is not intended to indicate the markers are only used to provide a prognosis or diagnosis of AD.

The methods for “aiding diagnosis” as used herein refer to methods that assist in making a clinical or near clinical determination regarding the presence, or nature, of AD or a neurological disease, and may or may not be conclusive with respect to the definitive diagnosis. As an example, a method of aiding diagnosis of AD may comprise measuring the amount of one or more biomarkers, as herein described, from a biological sample obtained from an individual.

In another example, a method of aiding diagnosis of AD may comprise measuring the amount of one or more AD biomarkers correlative to the presence of AD in a biological sample obtained from an individual. In a further example, a method of aiding diagnosis of a neurological condition according to the present invention can be used in combination with other methods of clinical assessment of a neurological disease, including, but not limited to, memory and/or psychological tests, assessment of language impairment and/or other focal cognitive deficits (such as apraxia, acalculia and left-right disorientation), assessment of impaired judgment and general problem-solving difficulties, assessment of personality changes ranging from progressive passivity to marked agitation.

The methods for “aiding prognosis” as used herein refer to methods that assist in making an assessment of a pre-clinical determination regarding the presence, or nature, of the AD, and may or may not be conclusive with respect to the definitive diagnosis. As an example, a method of aiding the prognosis of AD can comprise measuring the amounts or values of biomarkers correlative to the presence of AD in a biological sample obtained from an individual and utilising the presence of these biomarkers to ascertain the likelihood that an individual will have or will develop AD. In another example, a method of aiding prognosis of a neurological condition (such as AD) according to the present invention can be used in combination with other methods of clinical assessment of a neurological disease, including, but not limited to, memory and/or psychological tests, assessment of impaired judgment and general problem-solving difficulties, assessment of personality changes ranging from progressive passivity to marked agitation. The diagnosis can be then validated or confirmed if warranted, such as through imaging using PET, MRI, etc.

As used herein, the term “stratifying” typically refers to sorting individuals into different classes or strata based on the features of a neurological disease. For example, stratifying a population of individuals with a neurological disease involves assigning the individuals on the basis of the severity of the disease (e.g., mild, moderate, advanced, etc.).

The term “predicting” as used herein, refers to making a finding that an individual has a significantly enhanced probability of developing a neurological disease (such as AD).

The term “biological sample” as used herein, typically refers to a variety of sample types obtained from an individual that can be used in a prognostic, diagnostic or monitoring assay and includes, but is not limited to, blood (including whole blood), plasma or serum, urine, cerebrospinal fluid, tears or saliva. A blood sample may include, for example, various cell types present in the blood including platelets, lymphocytes, polymorphonuclear cells, macrophages, erythrocytes. In some embodiments of the present invention, the biomarker can be selected from any of those listed in TABLE 8. The term also includes samples that have been manipulated in any way after their procurement, such as by treatment with reagents, solubilisation, or enrichment for certain components, such as proteins or polynucleotides.

The term “biological” as used herein encompasses a variety of fluid sample types obtained from an individual and can be used in a diagnostic or monitoring assay. The definition encompasses blood, peripheral blood, platelets, serum and plasma. The definition also includes samples that have been manipulated in any way after their procurement, such as by treatment with reagents, solubilisation, or enrichment for certain components, such as proteins or polynucleotides. The term is generally considered to also refer to all fluids that contain or are suspected to contain biologically relevant molecules which may act as biomarkers (including, but not limited to, proteins, polypeptides, oligopeptides, polynucleotides, oligonucleotides or fragments thereof, nucleic acids, steroids or steroid hormones, sugars/carbohydrates, lipids, metals, other small molecules and cells) as ligand(s) described in the present invention. The biological may be a solution containing multiple known or unknown ligand(s) or a mixture containing multiple known or unknown ligand(s). Typical examples of biologicals include body fluids selected from blood, blood plasma, blood serum, hemolysate and the like. Additional examples of “biological” include medium supernatants of culture cells, tissue, bacteria and viruses as well as lysates obtained from cells, tissue, bacteria or viruses. Cells and tissue can be derived from any single-celled or multi-celled organism described above.

A “blood sample” is a biological sample which is derived from blood, preferably (or circulating) blood. A blood sample may be, for example, whole blood, plasma or serum.

The term “amyloid load” or “amyloid loading” (used interchangeably) refers to the concentration or level of cerebral amyloid-13 peptide (Aβ or amyloid-beta) deposited in the brain, amyloid-13 peptide being the major constituent of (senile) plaques.

Histopathological studies infer that the cerebral amyloid load gradually increases during the preclinical and clinical course of AD. The build up amyloid-beta affects neocortical areas first then spreads to allocortical areas such as gyrus cinguli and amygdala, later also involves diencephalic nuclei including thalamus and striatum, and finally extends to the brainstem and the cerebellum. In the measurement of this peptide, imaging agents are commonly used. In a preferred embodiment the imaging agent utilised is a radio tracer, and in a particularly preferred embodiment the radiotracer adopted is PiB. There have been numerous studies that have correlated the radio tracer signal or output with the level of Aβ and this has lead to the terminology of PiB positive and PiB negative. Typically the normalisation of the PiB output, or uptake of the tracer, occurs to allow inter- and intra-subject comparisons to be made. In clinical practice normalisation for the radioactive dose and the patients mass or volume (otherwise known as the standard uptake value (SUV)), is performed. The normalisation also incorporates standardisation with the (usually) unaffected cerebellum to provide the standard uptake value ratio (SUVR). This has led to the determination of a threshold value to differentiate those with high neocortical load (PiB positive) from those with a low load (PiB negative). A threshold of 1.5 has been proposed as an accepted “cut off” or “limit” value that appears to correlate well with a diseased state, although alternative threshold values may be more suitable for individual studies.

An “individual” is a mammal, more preferably a human. Mammals include, but are not limited to, humans, primates, farm animals, sport animals, rodents and pets. It is further considered that the term “individual” and “subject” can be used interchangeably to refer to the same test subject being examined or analysed for the presence of biomarkers and evaluated for determining the status of a neurological disease, such as AD.

The term a “Normal” individual or sample from a “Normal” individual as used herein for quantitative and qualitative data is generally considered to refer to an individual who has or would be assessed by a physician as not having AD, and has an Mini-Mental State Examination (MMSE) (referenced in Folstein et al 1975) score or would achieve a MMSE score in the range of 25-30. A “Normal” individual is generally age-matched within a range of 5 to 10 years, including but not limited to an individual that is age-matched, with the individual to be assessed.

The term “Questionable AD” in association with an individual as used herein is generally considered as an individual who (a) has been diagnosed with AD or has been given a diagnosis of probable AD, and (b) has either been assessed with the Mini-Mental State Examination (MMSE) (referenced in Folstein et al 1975) and scored 25-28 or would achieve a MMSE score of 25-28 upon MMSE testing. Accordingly, “Questionable AD” refers to AD in an individual having scored 25-28 on the MMSE and or would achieve a MMSE score of 25-28 upon MMSE testing.

The term “individual with mild AD” is generally considered as an individual who (a) has been diagnosed with AD or has been given a diagnosis of probable AD, and (b) has either been assessed with the Mini-Mental State Examination (MMSE) (referenced in Folstein et al 1975) and scored 22-27 or would achieve a MMSE score of 22-27 upon MMSE testing. Accordingly, “mild AD” refers to AD in an individual who has either been assessed with the MMSE and MMSE score of 22-27 or would achieve a MMSE score of 22-27 upon MMSE testing. In some embodiments, the MMSE score range for “mild AD” is 20-25.

In general, an “individual with moderate AD” is an individual who (a) has been diagnosed with AD or has been given a diagnosis of probable AD, and (b) has either been assessed with the MMSE and scored 16-21 or would achieve a MMSE score of 16-21 upon MMSE testing. Accordingly, “moderate AD” refers to AD in an individual who has either been assessed with the MMSE and scored 16-21 or would achieve a MMSE score of 16-21 upon MMSE testing. In some embodiments, the MMSE score range for “moderate AD” is 10-20.

In general, an “individual with severe AD” is an individual who (a) has been diagnosed with AD or has been given a diagnosis of probable AD, and (b) has either been assessed with the MMSE and scored 12-15 or would achieve a MMSE score of 12-15 upon MMSE testing. Accordingly, “severe AD” refers to AD in an individual who has been either assessed with the MMSE and scored 12-15 or would achieve a MMSE score of 12-15 upon MMSE testing. In some embodiments, the MMSE score range for “severe AD” is 10-20.

The term clinical marker values is used to denote clinical assessment carried out by a medical professional, or in some cases using programs on the internet. Assessments such as language impairment and/or other focal cognitive deficits (such as apraxia, acalculia and left-right disorientation), assessment of impaired judgment and general problem-solving difficulties, assessment of personality changes ranging from progressive passivity to marked agitation are all possible marker values. One regimented assessment is the Clinical Dementia Rating (CDR) which is a five-point scale in which CDR-0 connotes no cognitive impairment, and then the remaining four points are for various stages of dementia: CDR-0.5=very mild dementia, CDR-1=mild, CDR-2=moderate, CDR-3=severe. The information from which the CDR score is derived consists of a standard set of information collected in a clinical instrument. The six domains are: Memory, Orientation, Judgment and Problem solving, Community Affairs, Home and Hobbies, and Personal Care.

Other influential determinates of risk and that can be used to further enhance diagnosis, include age, gender, location sampled, community involvement, Body Mass Index, Marital Status, Years of Education, APOE Genotype, Anaemia Status, Physical Activity Quartile, Intra Cranial Volume, Hippocampal Volume, etc. As used herein, additional factors could be taken into account and use in the classification algorithm to increase sensitivity and/or specificity.

As used herein, the term “treatment” refers to the alleviation, amelioration, and/or stabilization of symptoms, as well as delay in progression of symptoms of a particular disease. For example, “treatment” of AD includes any one or more of: elimination of one or more symptoms of AD, reduction of one or more symptoms of AD, stabilization of the symptoms of AD (e.g., failure to progress to more advanced stages of AD), and delay in progression (i.e., worsening) of one or more symptoms of AD.

As used herein, a “reference value” can be an absolute value; a relative value; a value that has an upper and/or lower limit; a range of values; an average value; a median value, a mean value, a shrunken centroid value, or a value as compared to a particular control or baseline value. It is to be understood that other statistical variables may be used in determining the reference value. A reference value can be based on an individual sample value, such as for example, a value obtained from a sample from the individual with AD, but at an earlier point in time, or a value obtained from a sample from an AD patient other than the individual being tested, or a “normal” individual, that is an individual not diagnosed with AD. The reference value can be based on a large number of samples, such as from AD patients or normal individuals or based on a pool of samples including or excluding the sample to be tested.

The reference levels used for comparison with the measured levels for the biomarkers, such as AD biomarkers, from a subject may vary, depending on the aspect of the invention being practiced, as will be understood from the foregoing discussion. For identification of biomarkers indicative of a subject having AD, the reference level will be typically a predetermined reference level such as an average of levels obtained from a population that is not afflicted with AD, but in some instances, the reference level can be a mean or median level from a group of individuals including neurological disease patients. In some instances, the predetermined reference level is derived from (e.g., is the mean or median of) levels obtained from an age-matched population.

For AD prognostic methods, the “reference level” is typically a predetermined reference level, such as an average of levels obtained from a population that is afflicted with AD, but in some instances, the reference level can be a mean or median level from a group of individuals including AD patients. In some instances, the predetermined reference level is derived from (e.g., is the mean or median of) levels obtained from an age-matched population. In some examples disclosed herein, the age-matched population comprises individuals with non-AD neurodegenerative diseases.

For AD monitoring methods (e.g., methods of prognosing aiding in the diagnosis of AD progression in an AD patient), the reference level may be a predetermined level, such as an average of levels obtained from a population that is not afflicted with AD, a population that has been diagnosed with AD, and, in some instances, the reference level can be a mean or median level from a group of individuals including AD patients. Alternately, the reference level may be a historical reference level for the particular patient (e.g., an EGF level that was obtained from a sample derived from the same individual, but at an earlier point in time). In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or year increments.

As used herein, a “relevant coefficient” can be considered to be a value that is associated with one or more variables (biomarkers) that has been calculated based on the correlation of the variable with the response (neocortical amyloid loading). It may also relate to a set of values that are associated with a set of variables that have been calculated based on the correlation of the combined set of variables with the response. They thus define the relationship and correlation between the variables and the response and may be used in model generation and to provide a predictive evaluation of the response, given values for the variables.

### 3. Methods for Identifying Biomarkers in a Biological Sample

The present invention provides methods useful for determining a theoretical neocortical amyloid loading, which in turn is useful for prognosis, aiding prognosis, aiding diagnosis, assessing risk, the monitoring of a neurodegenerative disease, and/or predicting a neurodegenerative disease, in a subject through the use of identified biomarkers.

Biomarkers measured in the practice of the present invention may be, for example, any proteinaceous biological marker found in a biological sample of a subject. It is understood that a biomarker that is considered as “identified” is one that is useful for aiding in the prognosis, aiding in the diagnosis, monitoring, and/or prediction of a neurodegenerative disease, such as AD (or an AD-like disease) when it is significantly different between the subsets of biological samples tested.

In an aspect of the present invention, the presence of one or more biomarkers in biological samples from one or more subjects is determined. In one embodiment, the samples are selected such that they can be segregated into one or more subsets on the basis of a neurological disease, such as AD (e.g., samples from healthy individuals, those diagnosed with other dementias or diseases (as other dementia controls), or samples from individuals with AD).

In an embodiment of the present invention there are provided methods for assaying, detecting or measuring values for a set of biological samples from one or more individuals (where the samples can be segregated into one or more subsets on the basis of a neurological disease or another classification) are compared, wherein a biomarker set or profile or signature that vary are useful for the prognosis, aiding in diagnosis, monitoring, and/or prediction of the likelihood of a neurological disease. In a further embodiment, the biological samples obtained may be peripheral biological fluids.

In a further embodiment, the methods of the present invention include identifying a subject at risk of developing a neurodegenerative disease based on the presence of a panel of identified biomarkers. In a further embodiment, the method comprises determining the identity of at least one biomarker for indication of a neurological disease, where the disease is AD.

The methods of the present invention can be carried out by obtaining a set of measured values for a plurality of biomarkers from a set of biological samples, where the biological samples are divisible into at least two subsets in relation to the presence of a neurological disease, such as AD, comparing said measured values for each biomarker or biomarker set or signature that track with a particular attribute, such as amyloid loading. In a further embodiment, the biological samples examined for biomarkers may comprise predetermined validated samples from subjects that may have been investigated for the presence of a neurological disease. In still a further embodiment, the measured values for a plurality of biomarkers may be obtained from predetermined validated samples from subjects that have been investigated for the presence of a neurological disease, such as AD.

In determining or identifying the presence a biomarker from a biological sample, it is considered that many different assays or methodologies may be generally utilised which the skilled addressee may be well versed. Whilst some assay formats will allow testing of biological samples without prior processing of the sample, it is also possible that biological samples could be processed prior to testing. Processing can take the form of elimination of cells (nucleated and non-nucleated), such as erythrocytes, leukocytes, and platelets in blood samples, and may also include the elimination of certain proteins, such as certain clotting cascade proteins from blood or other standard means of protein separation, enrichment, or purification. In an example of prior processing, the biological sample is collected in a container comprising EDTA.

In a further aspect of the present invention, there is provided a set of biological samples, such as blood samples, that are derived from one or more individuals that can be analysed to determine the likelihood a subject will develop AD. The set of biological samples to be analysed can be selected such that they can be divided into one or more subsets on the basis of AD or another basis of classification. The division into subsets can be on the basis of presence/absence of disease or subclassification of disease (e.g., relapsing/remitting vs. progressive relapsing). In one embodiment, the samples may be selected such that they can be divided into groups where one of the groups can be based on the likely presence of absence of AD following evaluation of a subject or an individual through either clinical analysis or any other methodology such as MRI and/or PET scanning techniques. In a further embodiment, biomarkers measured in the practice of the invention may be any proteinaceous biomarkers found in a biological sample.

In a further embodiment, the biological samples may include those obtained from subjects suspected of having AD and which may have been evaluated via other clinical means for determining the presence of AD. In a further embodiment, the biological samples may include those obtained from individuals diagnosed as having high neocortical amyloid loading. In a further embodiment, the biological samples are obtained from individuals diagnosed as having high levels of amyloid beta loading. In a further embodiment, in addition to those samples from individuals diagnosed as having high levels of amyloid beta loading, other samples are included to provide a mixture from individuals that are normal or healthy and which may not possess high levels of amyloid beta.

In a further aspect, the present invention provides methods for the identification of at least one biomarker useful for the prediction or prognosis of an individual having a neurological disease, such as AD, by obtaining measured values of a plurality of biomarkers from set of biological samples obtained from at least one individual. In one embodiment, the relationship between the biomarkers in biological samples are compared to the measured values of a reference set of biomarkers that have been determined as being indicative of a subject having or likely to develop AD.

In a further aspect of the present invention, the inventors have determined that a signature based on the presence of a plurality of biomarkers, two of which are Amyloidβ42 and ApolipoproteinE (Genotype) and their naturally occurring variants or fragments thereof in a biological sample from an individual can be characteristic of an individual having or likely to develop a neurodegenerative disease, such as AD, and can provide an indication of the presence or likely predisposition for a test subject developing a neurodegenerative disease, such as AD.

In an embodiment of the present invention, the inventors have determined that a signature based on the presence of a plurality of biomarkers, two of which can be comprised of Amyloidβ42 and ApolipoproteinE (Genotype), when combined with at least one more selected from the group comprising cortisol, IgM, IL-17, PPY, VCAM1, BLC and their naturally occurring variants thereof or fragments thereof in a biological sample from an individual can be characteristic of an individual having or likely to develop a neurodegenerative disease, such as AD, and can provide an indication of the presence or likely predisposition for a test subject developing a neurodegenerative disease, such as AD.

In a further embodiment, the inventors have determined that a signature based on the presence of a plurality of biomarkers, two of which can be comprised of Amyloidβ42 and ApolipoproteinE (Genotype) when combined with at least one further marker as provided in TABLE 8 and their naturally occurring variants thereof or fragments thereof in a biological sample from an individual can be characteristic of an individual having or likely to develop a neurodegenerative disease, such as AD, and can provide an indication of the presence or likely predisposition for a test subject developing a neurodegenerative disease, such as AD.

In determining the presence of biomarkers in the biological sample from a subject, the sample may be divided into a number of aliquots, with separate aliquots used to measure different biomarkers (although division of the biological sample into multiple aliquots to allow multiple determinations of the levels of the biomarkers in a particular sample are also contemplated). Alternately the biological sample (or an aliquot there from) may be tested to determine the levels of multiple biomarkers in a single reaction using an assay capable of measuring the individual levels of different biomarkers in a single assay, such as an array-type assay or assay utilizing multiplexed detection technology (e.g., an assay utilising detection reagents labelled with different fluorescent dye markers).

In examining for the presence of a biomarker in a biological sample, it is common in the art to perform ‘replicate’ measurements when measuring biomarkers. Replicate measurements are ordinarily obtained by splitting a sample into multiple aliquots, and separately measuring the biomarker(s) in separate reactions of the same assay system. Replicate measurements are not necessary to the methods of the invention, but embodiments of the invention are capable of utilising replicate testing, particularly duplicate and triplicate testing.

It is considered that determining the presence of a biomarker in a biological sample can also be provided through many measurement techniques known in the art, such as affinity-based technologies, that utilise a molecule that specifically binds to a target, such as an AD biomarker to be measured as described herein. Such affinity molecules may be considered as an “affinity reagent,” and may include molecules such as antibodies or aptamers. It is considered however that other technologies, such as for example, spectroscopy-based technologies (e.g., matrix-assisted laser desorption ionization-time of flight (MALDI-TOF) spectroscopy) or assays measuring bioactivity (e.g., assays measuring mitogenicity of growth factors) may also be utilised and considered able to be used to determine the presence of a biomarker, such as biomarker for AD.

Affinity-based technologies can also be utilised to determine the presence of biomarkers and can be considered to further include antibody-based assays (immunoassays) and assays utilise aptamers (nucleic acid molecules which specifically bind to other molecules), such as ELONA (enzyme-linked oligonucleotide assay). Additionally, assays utilising both antibodies and aptamers are also contemplated for the purposes of the methods of the present invention (e.g., a sandwich format assay utilising an antibody for capture and an aptamer for detection) for identifying at least one AD biomarker.

A wide variety of affinity-based assays are known in the art. Affinity-based assays will utilise at least one epitope derived from the AD biomarker of interest, and many affinity-based assay formats utilise more than one epitope (e.g., two or more epitopes are involved in “sandwich” format assays; at least one epitope is used to capture the marker, and at least one different epitope is used to detect the marker).

It is further considered that affinity-based assays and technologies may be utilised in competition or direct reaction formats for the determination of the presence of a biomarker. Such assays would utilise sandwich-type formats, and may further be heterogeneous (e.g., utilise solid supports) or homogenous (e.g., take place in a single phase) and/or utilise immunoprecipitation. Most assays involve the use of labelled affinity reagent (e.g., antibody, polypeptide, or aptamer); the labels may be, for example, enzymatic, fluorescent, chemiluminescent, radioactive, or dye molecules. In another approach, all the proteins in the biological sample can be labelled using standard protein chemistry techniques and the labelled biomarkers are captured by the affinity reagents arrayed on a solid support. Assays which amplify the signals from the probe are also known; examples of which are assays which utilise biotin and avidin, and enzyme-labelled and mediated immunoassays, such as Enzyme-Linked Immunosorbent Assay (ELISA) and ELONA assays. As will be understood by the skilled addressee, either the biomarker or the reagent specific for the biomarker can be attached to a surface and levels can be measured directly or indirectly.

In a heterogeneous format, the assay may utilise two phases (typically aqueous liquid and solid). Typically an AD biomarker-specific affinity reagent is bound to a solid support to facilitate separation of the AD biomarker from the bulk of the biological sample. After reaction for a time sufficient to allow for formation of affinity reagent/AD biomarker complexes, the solid support or surface containing the antibody is typically washed prior to detection of bound polypeptides. The affinity reagent in the assay for measurement of AD biomarkers may be provided on a support (e.g., solid or semi-solid); alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, glass, Protein A beads, magnetic beads, and electrodes. Both standard and competitive formats for these assays are known in the art. Accordingly, in one embodiment, the determination of an AD biomarker may be as a complexes comprising a set of AD prognosis or diagnosis biomarkers as described herein that are bound to reagents specific for the biomarkers, wherein said reagents are attached to a surface. Also provided herein are complexes comprising a set of AD prognosis or diagnosis biomarkers as described herein that are bound to reagents specific for the biomarkers, wherein said biomarkers are attached to a surface.

It is further considered that sandwich antibody arrays may be used accordingly to the methods of the present invention. In one example, a high sensitivity multiplex sandwich ELISA can used in the methods of the invention to analyse biomarkers present in a biological sample from a subject.

In a homogeneous format for the detection of biomarkers, the assay takes place in single phase (e.g., aqueous liquid phase). Typically, the biological sample is incubated with an affinity reagent specific for the biomarker in solution. For example, it may be under conditions that will precipitate any affinity reagent/antibody complexes that are formed. Both standard and competitive formats for these assays are known in the art.

In a further example, a glass array platform that utilises indirect fluorescence detection may be used to analyse for one or more biomarkers for AD. In a further example, the one or more biomarkers analysed with the glass array platform that utilises indirect fluorescence detection are used to determine both biomarkers for AD and control biomarkers.

It is considered that complexes formed involving a biomarker and an affinity reagent can be detected by any of a number of known techniques known in the art, depending on the format of the assay and the preference of the user. For example, unlabelled affinity reagents may be detected with DNA amplification technology (e.g., for aptamers and DNA-labelled antibodies) or labelled “secondary” antibodies that bind the affinity reagent. In a further example, the affinity reagent may be labelled, and the amount of complex may be determined directly (as for dye-(fluorescent or visible), bead-, or enzyme-labelled affinity reagent) or indirectly (as for affinity reagents “tagged” with biotin, expression tags, and the like).

### 4. Measuring Values of Biomarkers

The measured level for a biomarker may be a primary measurement of the level a particular biomarker and therefore a measurement of the quantity of biomarker itself, (such as by detecting the number of biomarker molecules in the sample) or it may be a secondary measurement of the biomarker (a measurement from which the quantity of the biomarker can be determined but not necessarily deduced (qualitative data), such as a measure of enzymatic activity (when the biomarker is an enzyme) or a measure of mRNA coding for the biomarker). Qualitative data may also be derived or obtained from primary measurements.

Commercial kits, for example, are available that could be utilised in the methods of the present invention to measure multiplex proteins and panels of biomarkers. Multiplex MAPs (Millipore) which has two combinations of (I) alpha-syncline, Nerve Growth factor, beta subunit, neuron-specific enlace (NSE), Parkinson disease protein 5, Parkinson disease 7, and Tau—using CSF (ii) alpha-1 acid glycoprotein, ceruloplasmin, haptoglobuin, serum amyloid P component—using serum, plasma or CSF, may be utilised. The commercial kit from Proteome Sciences Plasma 9-Plex panel that consists of alpha2macroglobulin, apolipoprotein E, clusterin alpha, and beta, serum amyloid protein (SAP), complement C3, Complement factor H, gamma fibrinogen, gelsolin, as wells as a 3 panel assay that consists of cystain C, TBC, neurosecretory protein VGF for CSF samples, could also be utilised in the methods of the present invention.

As described herein, the levels of a set of biomarkers are measured in a biological sample from an individual. The biomarkers capable of providing an indication of an individual having or likely to develop a neurological disease, such as AD, can be measured by any methods as herein disclosed. The biomarker levels may be measured using any available measurement technology capable of specifically determining the levels of the biomarkers in a biological sample obtained from a subject or individual to be tested. The measurement may be either quantitative or qualitative, so long as the measurement is capable of indicating whether the level of each biomarker in the biological sample is above or below a reference value for that biomarker.

Typically the level of each marker in a test sample obtained from a subject can be determined using immunohistochemistry or immunoassay techniques, such as for example an enzyme immunoassay (EIA), and for which kits are readily commercially available from a number of commercial suppliers. Alternatively, hybridization techniques including PCR or a mass spectrometric platform may be used to determine the level of each marker in a test sample. The assay may involve a multiplex technique so the levels of two or more markers can be determined from the output of a single assay process.

In a further aspect of the present invention, the level of at least one biomarker is determined for at least one biomarker from a set of identified biomarkers in a biological sample from one or more individuals. The samples are selected such that they can be segregated into one or more subsets on the basis of neurological disease or disease (e.g., samples from healthy individuals, those diagnosed with other dementias or diseases (as other dementia controls), or samples from individuals with a diagnosed neurological disease). In some embodiments, the levels of a group of biomarkers are obtained for a set of biological samples from one or more individuals where the samples are segregated based on the diagnosed presence of a neurological disease in a subject, and where the subject is a healthy control. In a one embodiment, the disease is AD.

In a further embodiment, the marker signature arising from measurement of the levels of biomarkers in samples of a subject suspected of having AD and control samples of at least one of Amyloidβ42 and ApolipoproteinE (Genotype) when combined with at least one more biomarker selected from the group comprising cortisol, IgM, IL-17, PPY, VCAM1, BLC and naturally occurring variants or fragments thereof, are measured to produce measured values, wherein the biomarkers are useful for the prognosis, aiding diagnosis, monitoring, and/or predictive of AD, and can be indicative of likelihood of a subject developing AD or having an AD like disease.

In a further embodiment, the marker signature arising from measurement of the levels of biomarkers in samples of a subject suspected of having AD and control samples of at least one of Amyloidβ42 and ApolipoproteinE (Genotype) when combined with at least one further marker as provided in TABLE 8 and naturally occurring variants or fragments thereof, are measured to produce measured values, wherein the biomarkers are useful for the prognosis, aiding diagnosis, monitoring, and/or predictive of AD, and can be indicative of likelihood of a subject developing AD or having an AD like disease.

These biomarkers may form the basis of relevant variables or measured values for generating a biomarker signature of a patient to determine the likelihood of a subject developing AD or having an AD like disease. In one embodiment, the measured values of the biomarker signature can be used to generate a set of relevant coefficients to aid in predicting neocortical levels of amyloid beta.

In a further aspect of the present invention, the marker level of any two or more of the biomarkers in a test sample can be combined to produce a marker signature (sometimes referred to as a “biomarker profile”), that is characterised by a pattern composed of at least two or more marker levels. In one embodiment of the present invention, the biomarker profile can be composed of any combination of Amyloidβ42 and ApolipoproteinE (Genotype) when combined with at least one more from the group comprising cortisol, IgM, IL-17, PPY, VCAM1, BLC and their naturally occurring variants thereof.

In a further embodiment, with respect to a test sample provided from a subject to be examined for relevant biomarkers, a biomarker signature having a predetermined pattern, i.e., satisfying certain criteria such as minimum fold changes in level between AD and control samples, is indicative of AD relative to a marker signature lacking the predetermined pattern. In a further embodiment of the invention, levels of a set of biomarkers from biological samples from one or more individuals (where the samples can be segregated into one or more subsets on the basis of AD) are measured to produce measured values, wherein biomarkers that vary significantly are useful for prognosis, aiding in the diagnosis, monitoring, and/or predictive of AD of a subject.

According to the methods of the present invention, biomarker levels can be measured according to methods known in the art and available to the person skilled in the art and according to the methods as described herein, such as by using affinity-based measurement technologies. As considered herein, “affinity” is a term well understood in the art and it is viewed as the extent, or strength, of binding of a given reagent to further target. For example, this may be considered as the strength of binding of, an antibody to a binding partner, such as a biomarker for prognosis or a biomarker for diagnosis as described herein (or an epitope thereof). As will be understood by a skilled addressee, affinity may be measured and/or expressed in a number of ways known in the art, including, but not limited to, equilibrium dissociation constant (K_{D} or K_{d}), apparent equilibrium dissociation constant (K_{D} or K_{d}′), and IC₅₀ (amount needed to effect 50% inhibition in a competition assay; used interchangeably herein with “I₅₀”).

It may be further considered that array-type heterogeneous assays may be suitable for measuring levels of biomarkers according to the methods of the present invention where in one embodiment there is the measurement of multiple biomarkers. Array-type assays used in the practice of the methods of the invention will commonly utilise a solid substrate with two or more capture reagents specific for different biomarkers bound to the substrate a predetermined pattern (e.g., a grid). The biological sample is applied to the substrate and biomarkers in the sample are bound by the capture reagents. After removal of the sample (and appropriate washing), the bound biomarkers are detected using a mixture of appropriate detection reagents that specifically bind the various biomarkers. Binding of the detection reagent may be accomplished using a visual system, such as a fluorescent dye-based system. In one embodiment, the capture reagents may be arranged on the substrate in a predetermined pattern, and accordingly the array-type assays provide the advantage of detection of multiple biomarkers without the need for a multiplexed detection system.

In a further embodiment, the reagents may be selected so that they bind specifically the biomarkers identified in the present invention that form a marker signature that is indicative of presence or likelihood of a subject developing a neurological disease, such as AD. In still a further embodiment, the reagents selected may be provided as a kit for detecting the presence in a sample of the identified biomarkers that form the marker signature and may be indicative of presence or likelihood of a subject developing a neurological disease, such as AD. In yet a further embodiment, the kit may provide for the detection of identified biomarkers that may provide an indication or prediction of the amyloid beta loading.

As will be understood by a person skilled in the art, the mode of detection of the signal in determining the level of the biomarker will depend on the exact detection system utilised in the assay. For example, if a radiolabeled detection reagent is utilised, the signal will be measured using a technology capable of quantitatifying the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

In application of the methods of the invention, if immunoassay technologies are employed, any immunoassay technology that can quantitatively or qualitatively measure the level of an biomarker in a biological sample may be used. Suitable immunoassay technology includes radioimmunoassay, immunofluorescent assay, enzyme immunoassay, chemiluminescent assay, ELISA, immuno-PCR, immuno-infrared, and western blot assay.

Likewise, aptamer-based assays that can quantitatively or qualitatively measure the level of a biomarker in a biological sample may be used in application of the methods of the present invention. Generally, aptamers may be substituted for antibodies in nearly all formats of immunoassay, although aptamers allow additional assay formats (such as amplification of bound aptamers using nucleic acid amplification technology such as PCR or isothermal amplification with composite primers.

In a further example of a technique to measure the level of an AD biomarker, in using a standard (direct reaction) format, the level of biomarker/affinity reagent complex is directly monitored. This may be accomplished by, for example, determining the amount of a labelled detection reagent that forms is bound to biomarker/affinity reagent complexes. In a competitive format, the amount of biomarker in the sample is deduced by monitoring the competitive effect on the binding of a known amount of labelled biomarker (or other competing ligand) in the complex. Amounts of binding or complex formation can be determined either qualitatively or quantitatively.

In a further aspect of the present invention, there are provided are peptides, polypeptides, proteins, oligonucleotides or fragments thereof, or other reagents and kits comprising the peptides, polypeptides, proteins, oligonucleotides or fragments thereof that may act as reagents that may be used to determine the identity of the biomarkers that are likely to indicate that a subject possesses AD or is likely to develop AD. In one embodiment the marker level of any two or more of the biomarkers present in the kit when used against a test sample can produce a biomarker profile that is characterised by a pattern composed of at least two or more marker levels. In a further embodiment, the biomarker profile or signature of a sample can be determined using reagents that specifically detect the identified biomarkers comprise the marker profile or signature. In a further embodiment, the reagents can bind to any of the biomarkers identified according to the methods of the present invention that are obtained to indicate that a subject is likely to possess or develop a neurological disease, such as AD.

In a further embodiment of the present invention, peptides, polypeptides, proteins, oligonucleotides or fragments thereof, or other reagents and kits comprising the peptides, polypeptides, proteins, oligonucleotides or fragments thereof that may act as reagents that may be used to determine the identity of the biomarkers.

The biomarker profile obtained when using the kit can be composed of any combination of Amyloidβ42 and ApolipoproteinE (Genotype) when combined with at least one more from the group comprising cortisol, IgM, IL-17, PPY, VCAM1, BLC and their naturally occurring variants or fragments thereof.

In a further embodiment, the biomarker profile obtained when using the kit can be composed of any combination of Amyloidβ42 and ApolipoproteinE (Genotype) when combined with at least one further marker as provided in TABLE 8 and naturally occurring variants or fragments thereof, are measured to produce measured values, wherein the biomarkers are useful for the prognosis, aiding diagnosis, monitoring, and/or predictive of AD, and can be indicative of likelihood of a subject developing AD or having an AD like disease.

In a further embodiment, the aforementioned biomarkers can be used in accordance with any of the methods as herein described for the prognosis or aiding in the detection of a neurological disease. In a preferred embodiment, the aforementioned biomarkers can be used in accordance with the methods of the present invention to prognose or aid in the diagnosis and/or monitoring of a disease state where the disease is AD.

In a further aspect of the present invention, measuring the level for each biomarker forming the marker signature or profile in providing a prognosis or aiding in the diagnosis of a neurological disease, such as AD, may further comprise inputting the values of the detected biomarkers into a system pre-calibrated with a set of relevant coefficients that can aid in providing an indication of the presence or absence of a neurological disease. In an embodiment, the measured values may be compared with respect to a set of relevant coefficients where the relevant coefficients may provide a predictive indication of level of amyloid beta in a sample or in a subject. In a further embodiment, the level of amyloid beta may be an indication that an individual may develop a neurological disease, such as AD. In a further embodiment, an indication of the level of amyloid beta as provided by a set of relevant coefficients may be predictive of the neocortical levels of amyloid beta.

In some embodiments of the present invention, also provided herein are computer readable formats comprising values obtained by the methods as herein described.

### 5. Statistical Analysis of Identified Biomarkers

The present invention also provides methods for identifying one or more biomarkers useful for the prognosis and/or monitoring a neurological disease such as AD and/or stratifying an individual (i.e., sorting an individual with a probable diagnosis of a neurological disease or diagnosed with a neurological disease into different classes of the disease) through application of statistical analysis.

The usefulness of an identified biomarker for determining a disease status is considered “statistically significant” when the probability that the particular biomarker has been identified by chance is less than a predetermined value. The method of calculating such probability will depend on the exact method utilised to compare the levels between the subsets (e.g., if SAM is used, the q-value will give the probability of misidentification, and the p value will give the probability if the t test (or similar statistical analysis) is used). Alternatively, Random Forests classification and the Gini index may be used in determining whether a biomarker is statistically relevant in the methods of the present invention. For each split of a node, based on a specific variable (biomarker) in the Random Forest the Gini impurity criterion for the two resulting nodes is less than the impurity criterion of the parent node. Summing the Gini impurity criterion decreases that happen at every split in the forest based on a specific variable provides an importance measure for that variable. Comparison of these importance measures allows the most important variables to be selected. As will be understood by a person skilled in the art, the predetermined value will vary depending on the number of biomarkers measured per sample and the number of samples utilised. For example, a predetermined value to which a measured biomarker may be compared against may range from as high as 50% to as low as 20, 10, 5, 3, 2, or 1%.

There are a number of statistical tests for identifying biomarkers that vary significantly between the subsets, including the conventional t-test. As the number of biomarkers measured increases however, it is generally more convenient to use a more sophisticated technique, such as SAM (see Tusher et al., Proc. Natl. Acad. Sci. U.S.A. 98(9):5116-21, 2001) or Prediction Analysis of Microarray (PAM) (http://www-statstanford.edu.about.tibs/PAM/index.html), or Random Forests (Liaw A. and Wiener M., R News, 2(3), pp 18-22., 2002).

Further techniques that may be applied in the methods of the present invention and may be advantageous in assisting the determination of the statistical significance of biomarkers or models generated during identification may include Regression and Receiver Operating Characteristic (ROC) (Chem. 39(4), pp 561-577, M. H. and Campbell, C., 1993), Tree Harvesting (Hastie et al., Genome Biology 2001, 2: research 0003.1-0003.12), Self Organizing Maps (Kohonen, Biological Cybernetics 43(1):59-69, 1982), Frequent Item Set (Agrawal et al., Proc ACM SIGMOD pgs 207-216, 1993), Bayesian networks (Gottardo, et. al., Biostatistics (2001), 11, pp 1-372001), and the commercially available software packages CART and MARS.

Still further statistical classifiers that may be applied can further include SMO, Simple Logistic, Logistic, Multilayer Perceptron, Bayes Net, Naive Bayes, Naive Bayes Simple, Naive Bayes Up, IB1, lbk, Kstar, LWL, AdaBoost, ClassViaRegression, Decorate, Multiclass Classifier, Random Committee, j48, LMT, NBTree, Part and Ordinal Classifier.

For example, in application of the Random Forests technique the samples are split based on branching techniques, each split is based upon one of the biomarkers. Identification of a biomarker that may be statistically significant can then be determined and the importance score (as discussed herein) is assigned to each biomarker. Biomarkers with scores greater than an adjustable threshold are considered to be of interest and are thus identified.

In a further aspect of the present invention, there are provided methods of identifying at least one biomarker for use in prognosis, aiding in diagnosis and/or monitoring the progression of a neurological disease in an individual and/or stratifying an individual, the method comprising obtaining measured values from a set of biological samples from a number of individuals for a plurality of biomarkers, wherein the set of biological samples is divisible into subsets on the basis of whether a diagnosis has previously been reached on the biological sample for a neurological disease, comparing the measured values from each subset for at least one biomarker; and identifying at least one biomarker for which the measured values are significantly different between the subsets. In one embodiment, the neurological disease is AD. In a further embodiment, the measured values from each subset for at least one biomarker selected from Amyloidβ42 and ApolipoproteinE (Genotype) when combined with at least one more from the group comprising cortisol, IgM, IL-17, PPY, VCAM1, BLC.

and naturally occurring variants thereof, are compared from each subset for the at least one biomarker in order to identify whether the measured values for at least one biomarker indicate whether it is significantly different between the subsets. In a further embodiment, at least two of the aforementioned biomarkers are selected. In further embodiments, at least three or at least four or at least five of the aforementioned biomarkers are selected.

In a further embodiment, the measured levels between samples of a subject not diagnosed or evaluated as having AD and the control samples from individuals diagnosed as having AD for at least one of the following biomarkers Amyloidβ42 and ApolipoproteinE (Genotype) when combined with at least one more selected from the group comprising cortisol, IgM, IL-17, PPY, VCAM1, BLC. and naturally occurring variants thereof, are assayed to produce values, wherein biomarkers detected are useful for aiding in the prognosis, aiding diagnosis, monitoring, and/or predictive of AD, and can be indicative of likelihood of a subject developing AD. In a further embodiment, the measured values can be transformed through statistical analysis to generate a signature to assist in the prognostic determination or classification of the likelihood that an individual providing similar measured levels of the at least one biomarker identified in a biological sample have or develop AD.

In a further aspect of the present invention, there are provided methods of identifying a plurality of biomarkers for use in prognosis, aiding in diagnosis and/or monitoring progression of a neurological disease, in an individual and/or stratifying an individual, the method comprising obtaining measured values from a set of biological samples for a plurality of biomarkers, wherein the set of biological samples is divisible into subsets on the basis of whether the biological samples were obtained from subjects who had undergone a clinical evaluation of the presence of a neurological disease. In one embodiment, the biological samples are randomly divided into a plurality of groups for the purposes of cross-validation. In a preferred embodiment, the plurality of groups can be cross-validated according to the methods as described in the examples as provided herein. In a further embodiment, the measured levels between samples for cross-validation can be segregated based on whether they are derived from a subject not diagnosed as having AD and the control samples from individuals having AD for at least one of the following biomarkers Amyloidβ42 and ApolipoproteinE (Genotype) when combined with at least one more from the group comprising cortisol, IgM, IL-17, PPY, VCAM1, BLC and naturally occurring variants thereof, where the biomarker levels are measured to produce measured values, wherein biomarkers that vary significantly are useful for aiding in the prognosis, aiding diagnosis, monitoring, and/or predictive of AD, and can be indicative of likelihood of a non-clinically determined AD subject developing AD or having an AD like disease.

### 6. Methods of Generating a Model for the Predictive Assessment of AD

The changes in the level of any one or more biomarkers can be used to assess neocortical amyloid load to prognose, to aid in diagnosis of a neurological disease and/or to monitor a neurological disease in a subject (e.g., tracking disease progression in a subject or patient and/or tracking the effect of medical or surgical therapy). The changes in the level of any one or more biomarkers may also be evaluated statistically to generate a predictive model based a panel or set of biomarkers that can be utilised for extrapolation purposes to determine the likelihood that an individual will have or develop AD or an AD-like disease based on the presence of the levels or concentration of biomarkers present in the biological of the individual.

According to the methods of the present invention, the inventors have identified methods capable of identifying a plurality of biomarkers that may be present in a biological sample of an individual (e.g. blood, including serum or plasma) and which the marker profile or signature are altered in individuals with a neurological disease such as AD and through evaluation of the concentration or levels of the panel of biomarkers identified, methods for predicting whether an individual is likely to test positive to the presence of a neurological disease or develop a neurological disease, such as AD.

In a further aspect of present invention, there are provided methods for identifying at least one biomarker useful for prognosis or aiding in the diagnosis of a neurological disease in an individual and/or monitoring progression of a neurological disease in an individual and/or stratifying a patient (i.e., sorting an individual with a probable diagnosis of a neurological disease or diagnosed with a neurological disease into different classes of the disease), the method comprising obtaining measured values from a set of biological samples for a plurality of biomarkers, wherein the set of biological samples is divisible into subsets on the basis of a neurological disease or stratified based on the severity of a neurological disease, comparing the measured values for at least one biomarker; and identifying at least one biomarker for which the measured values differ (e.g., are significantly different between subsets), and generating a model using a statistical predictive system that is based on the measured valued of a set of biological samples from a subject diagnosed with a neurological disease, and using the model, extrapolate to determine whether an individual possessing similar biomarkers will be likely to test positive or be diagnosed as having or developing a neurological disease In some embodiments, the comparing process is carried out using Random Forests In some embodiments, the neurological disease is AD.

Ideally, the classification algorithm is selected from the group comprising Variable Importance Measures, Linear Discriminant Analysis (LDA), Diagonal Linear Discriminant Analysis (DLDA), Diagonal Quadratic Discriminant Analysis (DQDA), SAM, Random Forests (RF), Support Vector Machines (SVM), Support Vector Regression (SVR), Neural Network, k-Nearest Neighbour method, Analysis of Covariance (ANCOVA).

It is considered in the methods of the present invention, that sets of samples for analysis of biomarkers can be selected such that they can be divided into one or more subsets on the basis of a neurological disease or stratified based on the severity of a neurological disease. In the methods of the present invention, the division into subsets can be on the basis of presence/absence of disease, stratification of disease, or subclassification of disease (e.g., relapsing/remitting vs. progressive relapsing).

Through the methods of the present invention, there are also provided methods for the generation of models that are capable of being formed based on the presence of a panel of biomarkers in an individual and which can be used to predict the neocortical amyloid load of an individual and therefore whether the individual is likely to possess or develop AD or and AD-like disease.

A further aspect of the present invention involves cross-validation on data composed of clinically evaluated samples of a separate group of individuals identified as having a neurological disease, such as AD. In a further embodiment, the data that is cross-validated may form a subset of data that has initially been analysed in accordance with the methods of the present invention.

Accordingly, in a further aspect of the present invention, there are provided methods of identifying at least one biomarker which can be used to prognosis, aid in the diagnosis of a neurological disease, such as AD, through the predictive determination of the level of neocortical amyloid load (which in turn helps to stratify a neurological disease), where a set of measured values for a plurality of biomarkers is obtained from a set of biological samples, where the set of biological samples is divisible into at least two subsets in relation to a neurological disease, comparing said measured values between the subsets for each biomarker, and identifying biomarker signature profiles between the subsets. In one embodiment, the at least two subsets are defined on the basis of presence/absence of disease or stratification of a disease. In a further embodiment, at least one of the subsets forms a control set where the control set composed of measured values of biomarkers obtained from individuals evaluated as having a neurological disease. In a further embodiment, the neurological disease is AD.

In a further embodiment, the identified biomarkers are evaluated for a significant difference of the biomarker signature profile between the subsets by any statistical methods as described herein or as commonly known by the skilled addressee. In a further embodiment, the statistical model or set of relevant coefficients can be cross-validated using data obtained from measured values from individual(s) evaluated as having a neurological disease, such as AD. In a further embodiment, a test sample from a subject not having been provided a diagnosis for AD can be tested for the same biomarkers used in preparing a predictive model from control samples set composed of measured values of biomarkers obtained from individuals evaluated as having a neurological disease, and provided a prognosis for a neurodegenerative disease by comparing the results to the predictive model.

In a further embodiment, the individuals evaluated as having a neurological disease are those evaluated as having AD. In a further embodiment, the individuals evaluated as having AD are those that have been provided a positive diagnosis for AD with a radiotracer specifically recognising the presence of Aβ in brain tissue based on neocortical amyloid loading. In a preferred embodiment, the radiotracer recognising Aβ is PiB.

In a preferred embodiment, the measured levels between samples for cross-validation can be segregated based the control samples from individuals having AD for at least one of the biomarkers from the following panel of biomarkers Amyloidβ42 and ApolipoproteinE (Genotype) when combined with at least one more from the group comprising cortisol, IgM, IL-17, PPY, VCAM1, BLC and their naturally occurring variants thereof, and generating a statistical predictive model according to the methods as herein disclosed, measuring the same panel of biomarkers from a subject to be tested and determining the values of the same measured biomarkers, transforming the measured levels from the individual being tested to generate a model similar to that provided from the control samples, and from the model generated on the control samples and evaluate whether the individual is likely to possess or develop a neurological disease, such as AD.

These biomarkers may form the basis of relevant variables for generating a relevant signature of a patient to determine a likelihood of a subject developing AD or having an AD like disease. The identified biomarkers may form a set of relevant coefficients or a predictive model that may be utilised to determine a theoretical amount of neocortical amyloid loading in a subject.

In a further aspect, the present invention includes methods where at least two distinct data sets can be generated based on measured levels of a panel of identified biomarkers obtained from individuals, wherein the data sets are differentiated based on their composition and where the composition is determined based on whether or not a clinical or diagnostic evaluation has been reached for an individual to determine whether they possess a neurological disease, such as AD. In one embodiment, the data set providing a positive diagnosis for a neurological disease is composed of data obtained from individuals diagnosed as having AD. In a further embodiment, the data set obtained from individuals with a positive diagnosis for AD is provided from individuals that have been evaluated as having a high neocortical amyloid brain loading. In a further embodiment, the amyloid loading is measured by a radiotracer specifically recognising the presence of amyloid-beta in brain tissue. In a preferred embodiment, the radiotracer recognising amyloid-beta is PiB.

In a further aspect, the methods of the present invention are carried out by obtaining a set of measured values for a plurality of biomarkers from a set of biological samples, where the set of biological samples are obtained from at least one individual identified under clinical evaluation as being diagnosed to possess a neurological disease, such as AD, and where the set is further divisible into at least two subsets for the purposes generating data sets capable of forming a statistical predictive model for providing a system for the prognosis of neurological disease, such as AD, and where the model is applied to a panel of identified biomarkers measured in a biological sample from an individual not having undergone clinical diagnosis for a neurological disease and can predict the likelihood the individual will possess or develop a neurological disease, such as AD. In a further embodiment, a plurality of measured values or predetermined validated samples can be provided from individuals grouped as a cohort. In a further embodiment, the clinical diagnosis is based on the level of neocortical amyloid or amyloid loading present.

In some embodiments of the present invention, levels of a panel of biomarkers are obtained for a set of biological samples from one or more individuals. The samples are selected such that they can be segregated into one or more subsets on the basis of a neurological disease (e.g., samples from normal individuals and those diagnosed with amyotrophic lateral sclerosis or samples from individuals with mild AD and those with severe AD and/or other neurological diseases, such as neurodegenerative diseases).

In a further aspect, the methods of the present further comprises comparing the measured values from each subset as described herein for at least one biomarker and may further include non-biological markers into the statistical analysis. In one embodiment, the non-biological markers may be clinical markers, such as the age of individuals from which the set of biological samples was obtained, as herein described (see, e.g., Examples herein). In one embodiment, following comparing the age of the individuals, the subset is further compared with clinical marker values from the individuals. In a preferred embodiment, the clinical marker values include clinical values, such as Clinical Dementia Rating (CDR), Body Mass Index from which the set of biological samples was obtained, as herein described (see, e.g., Examples herein).

In a further aspect, the methods of the present further comprises comparing the measured values from each subset for the at least one biomarker from the following panel of Amyloidβ42 and ApolipoproteinE (Genotype) when combined with at least one more from the group comprising cortisol, IgM, IL-17, PPY, VCAM1, BLC and naturally occurring variants or fragments thereof, and further includes comparing clinical marker values of individuals such as, CDR or Body Mass Index or both from which the set of biological samples was obtained, as herein described (see, e.g., Examples herein). In one embodiment, a predictive model is generated based on the returned measured values.

In a further embodiment, following comparing the clinical marker values of individuals, the subset is further compared by examining the age of the individuals investigated, the demographic, the level of education, age, gender, location sampled, community involvement, Body Mass Index, Marital Status, Years of Education, APOE Genotype, Anaemia Status, Physical Activity Quartile, Intra Cranial Volume, and Hippocampal Volume.

In a further aspect, the present invention provides methods for generating a predictive model for the prognosis of a neurological disease, such as AD, of an individual by supplementing the measured values for biomarkers from test subjects with clinically evaluated markers determined from individuals evaluated as being clinically diagnosed or likely to possess a neurological disease, such as AD. In a further embodiment, the methods of the present further comprises comparing the measured values from each subset for the at least one biomarker as described herein and may further include comparing further clinical marker values of individuals such as CDR or Body Mass Index from which the set of biological samples was obtained.

In a further aspect, the present invention provides methods for preparing statistical models that are capable of being generated based on the presence of a panel of biomarkers that form a marker signature from a biological sample obtained an individual and that can be utilised to predict whether an individual is likely to possess or develop AD or and AD-like disease, where the model is generated based on data composed of a plurality of pre-validated samples of a separate group of individuals identified as having a neurological disease, such as AD. In one embodiment, the plurality of pre-validated samples of a separate group of individuals may be obtained through cohort study data of subjects or individuals for which biological fluid samples from these individuals has previously been assayed.

In a further embodiment, the present invention includes methods where the statistical model is prepared from at least two distinct data sets that are based on measured levels of a panel of identified biomarkers obtained from individuals, wherein the data sets are differentiated based on their composition and where the composition is determined on whether or not a clinical or diagnostic evaluation has been performed on an individual to determine whether they possess a neurological disease, such as AD. In a further embodiment, at least one biomarker is selected from the following panel of biomarkers Amyloidβ42 and ApolipoproteinE (Genotype) when combined with at least one further marker as provided in TABLE 8 and naturally occurring variants or fragments thereof. In one embodiment, at least one biomarker is selected from the following panel of biomarkers Amyloidβ42 and ApolipoproteinE (Genotype) when combined with at least one more from the group cortisol, IgM, IL-17, PPY, VCAM1, BLC. Amyloidβ42, Apolipoprotein E, Cortisol and/or BLC, IgM, IL-17, Pancreatic Polypeptide, and VCAM1 and their naturally occurring variants or fragments thereof.

In a further embodiment, the methods of the present further comprises comparing the measured values from each subset for the at least one biomarker as herein described and may further include comparing further clinical marker values of individuals such as CDR or Body Mass Index from which the set of biological samples was obtained and transforming the measured levels from the individual being tested to generate a statistical model to evaluate whether an individual is likely to possess or develop a neurological disease, such as AD. In a further embodiment, the statistical model is generated using control measurements of biomarkers from individuals diagnosed with AD according to any known clinical means known in the art.

In a further embodiment, the data set providing a positive diagnosis for a neurological disease is composed of data obtained from individuals diagnosed as having AD. In a further embodiment, the data set obtained from individuals with a positive diagnosis for AD is provided from individuals that have been evaluated with a radiotracer specifically recognising the presence of the Aβ in brain tissue. In a preferred embodiment, the radiotracer recognising the Aβ is PiB.

In further embodiments, the statistical methods utilised accordance with methods of the present invention comprises comparing the measured values from each subset for at least one biomarker by using Random Forest (RF). In some embodiments, the method provides sensitivity of at least 85% and specificity of at least 85% in providing a prognosis or in aiding in the diagnosis of a neurological disease such as AD in an individual.

### 7. Statistical Modelling

The process of comparing the measured values and evaluating data sets may be carried out by any method known in the art, including Random Forest (RF), Significance Analysis of Microarrays, Tree Harvesting, CART, MARS, Self Organizing Maps, Frequent Item Set, or Bayesian networks. In some embodiments of the present invention, the process of comparing the measured values is carried out by one or more of the statistical methods selected from the group consisting of Boosted Trees (BT), Linear Models for Micro Array data (LIMMA), Classification Trees (CT), Linear Discriminant Analysis (LDA), and Stepwise Logistic Regression, Shrunken centroids, Sparse Partial Least Squares, or Flexible Discriminating analysis.

Several different analysis approaches can be used to generate formulae or models that distinguish between AD and healthy control participants (or more specifically stratify participants based on disease severity according to amount of amyloid loading or those who had not been diagnosed with AD) on the basis of a small subset of their biomarker values and amyloid loading for use in the methods of the present invention. The use of multiple methods increases the robustness of the conclusions about the usefulness of the final set of biomarkers, since each method brings a different bias. Biomarkers selected by multiple methods are more likely to provide valid predictions. It may further be considered that cross-validation of data or the use of training-sets may provide methods to refine the statistical predictions and to further aid in determining which biomarkers from the plurality of predetermined validated samples may be selected to form the biomarker signature or profile that can aid in forming a set of relevant coefficients or predictive model to indicate the neocortical amyloid load.

In one example, a training set/test set approach was used so that the data used to fit the models was separate from that used to test their performance as predictors. The groups of AD cases and those AD cases that had been PET imaged with the PiB radio tracer were each divided into a training set and a test set, where the test set was not used for generation of the statistical model. The models were fitted to the training set and their performance evaluated on the test set using ROC. The fitting and testing was repeated numerous times, deemed necessary to refine the models, and based on the numerous splits of the data to generate numerous training and test datasets.

Many further statistical methods can then be used to identify a small subset of biomarkers giving good discrimination between the separate AD groups.

RF (classification) is a variable selection method that uses classification trees to infer class membership to each case. RF grows a number of classification trees (a forest), and counts the number of votes from trees (each tree provides a vote for a specific class) to predict class membership. RF outputs a variable importance, which is a relative measure on how well each variable is able to predict the class membership. Variable importance is plotted as the mean decrease in accuracy from each RF model. To compile a reduced list of useful biomarkers, and increase the accuracy of class prediction, multiple RF iterations after variable reduction, based upon variable importance, can be computed.

The LIMMA method has been widely used in the analysis of micro array data. Its general purpose to identify gene expression difference between two classes where P>>N (i.e., more variables than observations). The method typically starts with fitting a standard linear model to the data, and then uses an Empirical Bayes approach to borrow information across variables (reduction of sample error), and uses a moderated t-statistic with an augmented degrees of freedom. The LIMMA method outputs a False Discovery Rate (FDR) adjusted p-value (the q-value) which is useful in the relative difference between mean samples. The LIMMA method can be used to determine differences in mean biomarker level between HC and AD participants.

The CT method is an alternative approach to a non-linear regression where there are many complex interactions between multiple variables, whether they are continuous or categorical in nature. The method creates multiple partitions or subdivisions of data (recursive partitioning) so that the interaction between multiple variables becomes simpler. Recursive partitioning is analogous to creating multiple classification trees, where the interior branches are questions, and the outer leaves are the answers to the questions. Once simple partitions or trees have been formulated, simple local models are computed before outputting final tree structures, including criterions at which each branch (or variable) should be split by. This method has advantages in that (i) it allows one to see what variables are selected for the final tree in the model, and (ii) it allows for further biomarker analyses in combination with Receiver Operating Characteristic (ROC) analyses; integrating lifestyle, genetic markers and biomarkers to identify proportional AD risk.

BT (classification) is a variable selection and class prediction method that builds an initial binary classification tree (a root node and two child nodes), and then fits another tree based upon the partition residuals from the prior tree. This computation can be iterated many times, and acts as a weighted remodelling process prior to votes for class prediction are totaled from all trees. BT outputs a relative influence measure that, similar to the variable importance, provides a relative measure on how well each variable is able to predict class membership. The BT method also produces a predicted probability of class membership, which is useful for comparison of predicted class membership to actual class membership.

LDA is a statistical method that determines a linear combination of variables that separate two or more class groups.

Stepwise Logistic Regression is a statistical method whereby many predictor variables are added to a Logistic Regression framework, and multiple “steps” are taken to add/remove variables to decrease the error within the statistical model. In this way, the method accurately assesses each of the variables added into the model and determines how much each contributes to the prediction. Thus, using the biomarkers (including age) chosen from the RF, BT, LIMMA and CT methods as herein described, the Stepwise Logistic Regression can be performed and compared with the standard Logistic Regression.

### 8. Prognostic Determination of AD Based on AD Biomarkers

As previously herein discussed, a biomarker for AD or an AD biomarker is any protein or nucleotide or peptide marker that can be found and measured in sample from an individual, such as a blood sample, the level of which in the sample when compared to the level of the marker in a reference (control sample) that provides a reference level and is capable of being correlated with a prognosis of AD.

In one aspect, the levels of an identified number of biomarkers can form a marker signature or profile that can be used to predict the level of neocortical amyloid loading in a subject. In one embodiment, the level of neocortical amyloid loading can be determined by generating a set of relevant coefficients or a predictive model based on the identified biomarkers from a plurality of predetermined validated samples. In a further embodiment, non-biological markers derived from the individuals assayed in obtaining the plurality of predetermined validated samples can be inputted to the values for the identified biomarkers in generating a set of relevant coefficients or a predictive model for predicting the level of neocortical amyloid loading. In a further embodiment, the plurality of predetermined validated samples can be obtained from a cohort study of individuals. In a still further embodiment, the plurality of predetermined validated samples or cohort study can further include values from individuals clinically diagnosed as having a neurological disease, such as AD. In a still further embodiment, a subset of the individuals may have undergone imaging or analysis for neocortical amyloid loading through evaluation by radiotracer examination. In a preferred embodiment, the radiotracer examination may have been performed using PiB as known by those skilled in the art.

As will be understood in the practice of the AD prognosis methods of the present invention (i.e., methods of providing a prognosis or aiding in the prognosis of AD), if more than one AD prognosis biomarker is used, the evaluation of a prognosis of AD may vary and improve in sensitivity or specificity. For example, in some embodiments, when the method utilises five AD prognosis biomarkers the result would be considered as suggesting or indicating a percentage confidence of a particular level of successful prognosis of AD for the individual, whereas if six, seven, eight or nine markers composed of both biomarkers and non-biological makers (such as clinical markers) were utilised the percentage confidence (and thus sensitivity and specificity) may be increased as well as the likelihood in prognosing AD. In some embodiments, the overall pattern of the markers (e.g. how each marker compares with one or more sets of references levels) is used in prognosis of AD. Various algorithms, classifiers, and/or decision trees as described herein may be used to evaluate the overall levels of the biomarkers to determine a prognosis or assisting in the prognosis of AD.

As will be appreciated by those skilled in the art, methods disclosed herein may include the use of any of a variety of biological markers (which may or may not be AD markers) to determine the integrity and/or characteristics of the biological sample(s) (e.g. gender). Examples of further biological markers that could be evaluated are provided in TABLE 8.

AD prognosis can be determined or confirmed according to any one or more known clinical standards such as the clinical neuropsychology or behaviour assessments as known as recognized and used by health professionals. As described herein, the protein and peptide biomarkers Amyloidβ42 and ApolipoproteinE (Genotype) when combined with at least one more from the group comprising cortisol, IgM, IL-17, PPY, VCAM1, BLC and naturally occurring variants or fragments thereof, can be used to prognose or assist in the diagnosis of AD and are characterised by one or both of the following: 1) on an individual basis, the value of the biomarker in an AD subject is significantly different from that in a control sample (such as age-matched), and 2) the change in value of the biomarker in an AD subject relative to the equivalent control, is significant as an element of a biomarker signature consisting of multiple biomarkers, which together establish a pattern of change in values that is indicative of AD in a subject as compared to the pattern of expression observed for the same biomarkers in an appropriate control sample.

In the methods of the present invention, to classify a test sample as AD positive, or a subject as having AD, the value of at least one of the biomarkers is obtained and compared to a predictive model or a predetermined set of relevant coefficients. It will be understood that any number of individually significant biomarkers, for example any one or more of Amyloidβ42 and ApolipoproteinE (Genotype) when combined with at least one more from the group comprising cortisol, IgM, IL-17, PPY, VCAM1, BLC or those listed in TABLE 8 and their naturally occurring variants or fragments thereof, can be used.

In accordance with a further embodiment of the present invention, the concentrations in fluid, or other fluids of the biomarkers Amyloidβ42 and ApolipoproteinE (Genotype) when combined with at least one more from the group comprising cortisol, IgM, IL-17, PPY, VCAM1, BLC and naturally occurring variants or fragments thereof, as a composite, or collective, optionally further comprising the clinical markers CDR or Body Mass Index is more predictive than the absolute concentration of any individual marker in predicting clinical phenotypes, disease detection, monitoring, and treatment of AD. In addition, a further embodiment may in addition include the further clinical markers for evaluation could be selected from the group comprising Gender, Location Sampled, Community Involvement, Anaemia Status, Age, Marital Status, Years of Education, Physical Activity Quartile, Intra Cranial Volume, or Hippocampal Volume.

Analysis of the biomarker values may further involve comparing the values of at least two biomarkers with that of a predetermined predictive model or a set of relevant coefficients. In one embodiment, the set of relevant coefficients is obtained according to the methods as herein described. Classification analyses or algorithms can be readily applied to analysis of marker levels using a computer process. For example, a reference 3D contour plot can be generated that reflects the biomarker levels as described herein that correlate with a disease classification of AD. For any given subject, a comparable 3D plot can be generated and the plot compared to the reference 3D plot to determine whether the subject has a biomarker signature indicative of AD. Classification analysis, such as classification tree analyses are well-suited for analysing biomarker levels because they are especially amenable to graphical display and are easy to interpret. It will however be understood that any computer-based application can be used that compares multiple biomarker levels from two different subjects, or from a reference sample and a subject, and provides an output that indicates a disease classification of AD as described herein.

In various embodiments, the sensitivity achieved by the use of the set of biomarkers and/or clinical markers in a method for prognosing or aiding diagnosis of is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the specificity achieved by the use of the set of biomarkers in a method for prognosis or aiding diagnosis of AD is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the overall accuracy achieved by the use of the set of biomarkers in a method for prognosing or aiding diagnosis of AD is at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In some embodiments, the sensitivity and/or specificity are measured against a clinical diagnosis of AD.

In certain embodiments of the invention, levels for AD biomarkers are obtained from an individual at more than one time point. Such “serial” sampling is well suited for the aspects of the invention related to monitoring progression of AD in an AD patient. Serial sampling can be performed on any desired timeline, such as monthly, quarterly (i.e., every three months), semi-annually, annually, biennially, or less frequently. The comparison between the measured levels and the reference level may be carried out each time a new sample is measured, or the data relating to levels may be held for less frequent analysis.

As will be understood by those skilled in the art, biological samples including biological samples are usually collected from individuals who are suspected of having AD, or developing AD. The invention also contemplates samples from individuals for whom AD diagnosis is desired. Alternatively, individuals (or others involved in for example research and/or clinicians may desire such assessments without any indication of AD, suspected AD, or risk for AD. For example, a normal individual may desire such information. Such individuals are most commonly 65 years or older, although individuals from whom biological samples, such as biological samples are taken for use in the methods of the invention may be as young as 35 to 40 years old, when early onset AD or familial AD is suspected.

The invention also provides methods of screening for candidate agents for the treatment of AD by assaying prospective candidate agents for activity in modulating the set of AD biomarkers. The screening assay may be performed either in vitro and/or in vivo. Candidate agents identified in the screening methods described herein may be useful as therapeutic agents for the treatment of AD.

### 9. Qualification of the AD Status of a Subject

As described herein, the present invention provides methods for determining calculating a theoretical value for the neocortical amyloid load in a subject so to assist in predicting the status or likely status of a neurological disease, such as AD, in a subject (i.e. status: AD v. non-AD). The presence or absence of AD is determined by measuring the relevant biomarker or biomarkers and then either submitting the values to a classification algorithm with a set of relevant coefficients or predictive model. Here, the particular values of the pattern biomarker(s) of assayed correlates with the relevant coefficient or predictive model so to then associate the subject with the particular risk level of AD by predicting the neocortical amyloid load.

Accordingly, in a further aspect of the present invention, there is provided for an implementation of the methods as described herein in the form of a system, such as for example, a computer software program, which can be utilised by physicians and researchers to characterise and/or quantify a neurological disease, such as AD, for a subject or a group of subjects.

In an example of the application of a system utilising the methods of the present invention, for each subject, the information regarding the user (e.g. age, gender) is inputted in combination with a sample of the biological of the subject being assayed. The software then computes a score. In one example, the amyloid loading, may be returned as either PiB positive or PiB negative. Alternatively, the amyloid loading, normalised to SUVR scores, may be between 0.0 and 1.0. In a further example, the SUVR score may be either greater than 1.5 or less than 1.5 and which may indicate the likely status of AD in the assayed subject based on the calculated neocortical amyloid loading and which is based on the measured values of a panel of biomarkers that are present in the biological sample. In such an example, a SUVR score of <1.5 corresponds to a healthy person and SUVR score of 1.5 or higher to a person considered to be likely to have or to develop AD, taking into account the demographics of the subject such as age, gender, etc. In a further example, it may be conceivable that the threshold may be 1.5, or 1.4, or 1.3, depending on the appropriate circumstances for measurement or transformation of data. It is further considered that SUVR scores from PiB images may vary between, for example, 0.0 and 4.0, although in particular instances the data may be transformed so that the returned number varies between any desired range or number.

The scoring or PiB positivity or negativity can then be used either to help in further diagnosing the subject, to assess the efficacy of a treatment (the score should go down if the treatment is effective), or to compute the average score of a group of individuals in order to study a new therapy or a specific characteristic of the group (e.g. genetic mutation). In a further example, the efficacy of treatment may be assessed by the reduction of the SUVR score measured on a particular subject. This “AD score” reflects the progression of the subject towards AD. It provides a quantitative or close to quantitative assessment of a new subject at a single time point, and allows monitoring the disease progression on a given subject, or a population.

The biomarkers of the present invention can be used in prognostic tests to provide an assessment of the AD status in a subject, e.g., to diagnose AD disease. The phrase “AD status” includes distinguishing; inter alia, AD v. non-AD and, in particular, AD v. non-AD normal, MCI v. non-AD normal or AD v. MCI. Based on this status, further procedures may be indicated, including additional diagnostic tests or therapeutic procedures or regimens.

The power of a diagnostic or a prognostic model or test to correctly predict status is commonly measured as the sensitivity of the assay, the specificity of the assay or the area under a ROC curve. Sensitivity is the percentage of true positives that are predicted by a test to be positive, while specificity is the percentage of true negatives that are predicted by a test to be negative. An ROC curve provides the sensitivity of a test as a function of specificity. The greater the area under the ROC curve, the more powerful the predictive value of the test. Other useful measures of the utility of a test are positive predictive value and negative predictive value. Positive predictive value is the percentage of actual positives who test as positive. Negative predictive value is the percentage of actual negatives that test as negative.

The ROC method has been primarily used as a tool for the measurement of accuracy to define a criterion by which a certain markers can correctly classify a person into a designated class. ROC analyses provides multiple outcomes, one of which, the Area Under the Curve (AUC) is a useful measure for assessing model performance. The AUC statistic can be utilised within the biomarker analysis to compare Logistic Regression and Stepwise Logistic Regression models (e.g., from training set data) using different numbers of biomarkers. Sensitivity and specificity from statistical models computed on test set data can also be plotted to provide a graphical comparison of the performance of the models.

Accordingly, changes in the level of any one or more of these biomarkers from a biological sample from an individual can be used to assess cognitive function, to diagnose or aid in the prognosis or diagnosis of a neurological disease and/or to monitor a neurological disease in a patient (e.g., tracking disease progression in a patient and/or tracking the effect of medical or surgical therapy in the patient). Changes in the level of any one or more of these biomarkers can also be used to stratify a patient (i.e., sorting an individual with a probable diagnosis of a neurological disease or diagnosed with a neurological disease into different classes of the disease) and diagnosing or aiding in the diagnosis of mild cognitive impairment (MCI) as well as diagnosing or aiding in the diagnosis of cognitive impairment.

It would be understood by the skilled addressee that the degree of sensitivity and/or selectivity of the methods of the present invention in prognosis, aiding in diagnosis and/or monitoring an individual with a neurological disease and/or stratifying an individual, as herein described, will generally be greater where the method comprises comparing the measured value of all biomarkers in the biological sample.

Provided herein are methods for aiding diagnosis of AD by obtaining measured levels of sets of AD prognosis biomarkers in a biological sample from an individual, such as for example, a biological sample from an individual, and comparing those measured levels to reference levels, wherein the sets of biomarkers can comprise Amyloidβ42 and ApolipoproteinE (Genotype) when combined with at least one more selected from the group comprising cortisol, IgM, IL-17, PPY, VCAM1, BLC and naturally occurring variants or fragments thereof, or may comprise the clinical markers CDR or Body Mass Index or both, either set of which may optionally comprise additional biomarkers (e.g. one, two, three, or more additional biomarkers).

Methods of providing a prognosis or aiding in the diagnosis of AD as described herein may comprise any of the following steps of obtaining a biological sample from an individual, measuring the level of each biomarker in the set in the sample and comparing the measured value to an appropriate reference, such as a predetermined set of relevant coefficients; predicting an AD status based on comparison of measured values to the set of relevant coefficients. Comparing a measured value of a biomarker used for AD prognosis in a sample may be performed for each biomarker an identified set of biomarkers that may form a particular biomarker signature or profile indicative of neocortical amyloid loading. The present invention also provides methods of evaluating results of the analytical methods described herein. Such evaluation generally entails reviewing such results and can assist, for example, in advising regarding clinical and/or diagnostic follow-up and/or treatment options. The present invention also provides methods for assessing a biological sample for an indicator of any one or more of the following: AD; progression of AD; by measuring the level of or obtaining the measured value of or comparing a measured value of each biomarker in a set of biomarkers as described herein.

A further aspect of the present invention includes methods for assessing the efficacy of treatment modalities in individuals, or population(s) of individuals, such as from a single or multiple collection centre(s), diagnosed with AD or predicted to be at risk of converting to AD comprising any one of the following steps: obtaining a biological sample from the individual(s) subject to treatment; measuring the level of each biomarker in the set in the sample and comparing the measured level to an appropriate reference, which in some examples is a measured level of the biomarker in a fluid sample obtained from the individual(s) prior to treatment; obtaining measured levels of each biomarker in the set in a sample from the individual(s) and comparing the measured level to an appropriate reference; comparing measured levels of each biomarker in the set obtained from a sample from the individual(s) to an appropriate reference; measuring the level of each biomarker in the set in a sample from the individual(s); measuring the level of each biomarker in the set in a sample from the individual(s) and comparing the measured level to an appropriate reference; diagnosing efficacy of treatment based on comparison of measured levels to an appropriate reference; or obtaining a measured value for each biomarker in the set in a sample. Measured levels of each biomarker in the set may be obtained once or multiple times during assessment of the treatment modality.

In some embodiments, the biomarkers of the present invention can be used in combination with the age of an individual to aid in the prognosis, diagnosis and/or to diagnose a neurological disease in an individual, for example, as herein described (e.g., as described in the Examples section herein).

For methods providing a prognosis of AD as described herein, a reference level may also be considered as generally a predetermined level considered ‘normal’ for the particular AD diagnosis biomarker (e.g., an average level for age-matched individuals not diagnosed with AD or an average level for age-matched individuals diagnosed with neurological diseases other than AD and/or healthy age-matched individuals), although reference levels which are determined contemporaneously (e.g., a reference value that is derived from a pool of samples including the sample being tested) are also contemplated.

As described herein, assessment of results can depend on whether the data were obtained by the qualitative or quantitative methods described herein and/or type of reference point used. For example, qualitative measurement of AD biomarker levels relative to another reference level, which may be relative to the level of another AD biomarker, may be obtained. In other methods described herein, quantitative or absolute values, that is protein concentration levels, in a biological sample may be obtained. “Quantitative” result or data refers to an absolute value, which can include a concentration of a biomarker in pg/mL or ng/mL of molecule to sample. An example of a quantitative value is the measurement of concentration of protein levels directly for example by ELISA. “Qualitative” result or data provides a relative value which is as compared to a reference value. In some examples herein, qualitative measurements are assessed by signal intensity on a filter. In some examples herein, multiple antibodies specific for AD biomarkers are attached to a suitable surface, e.g. as slide or filter. Qualitative assessment of results may include normalizing data. In this disclosure, various sets of biomarkers are described. It is understood that the invention contemplates use of any of these sets.

### 10. Determining Risk of Developing Disease

In a further aspect of the present invention, there are provided methods for determining the risk of developing disease in a subject. Biomarker values or patterns are characteristic of various risk states, e.g., high, medium or low. The risk of developing a disease is determined by measuring the relevant biomarker or biomarkers and then submitting them to a classification algorithm. In one embodiment, the classification algorithm gives a formula to provide an indication of the amount of amyloid beta (β) present. In a further embodiment, the classification algorithm gives a functionality to provide an indication of the neocortical amyloid loading from a test sample of a subject.

In one aspect, this invention provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount, a set of relevant coefficients, and/or specific profile of biomarkers that is associated with the particular stage.

In one embodiment, the relevant biomarker or biomarkers are those that are identified as being indicative of a potential prognosis of AD or and AD-like disease. In a further embodiment, the biomarkers are compared against a classification algorithm or comparing them with a reference amount of biomarkers that have been obtained from cohort data or subject that has been identified and/or clinically diagnosed as having a neurological disease such as AD. In a particularly preferred embodiment, the set of relevant coefficients for the value of biomarkers have been obtained from individuals determined to have AD by clinical radiotracer diagnosis specific for Aβ. In a further embodiment, the set of relevant coefficients obtained provide an indication of neocortical amyloid loading that can provide a predictive interpretation of the likelihood a tested subject will develop AD or an AD-like disease.

In a further embodiment, the biomarkers being evaluated can be selected from at least any one of Amyloidβ42 and ApolipoproteinE (Genotype) when combined with at least one more from the group comprising cortisol, IgM, IL-17, PPY, VCAM1, BLC and their naturally occurring variants or fragments thereof, where the biomarker levels are measured to produce measured values which can be compared through a classification algorithm as described herein with values from equivalent reference biomarkers that have been obtained from cohort data or from a subject that has been identified and/or clinically diagnosed as AD.

In a further embodiment, the methods of the present additionally comprise including the further clinical marker values CDR or Body Mass Index or both, from which the set of biological samples was obtained and inputting these values with the classification algorithm to provide further increased sensitivity and selectivity of predicting the likelihood a subject will possess or develop AD.

### 11. Data Analysis

According to the present invention, it is considered that the methods herein described for the prognosis, aiding in diagnosis or likelihood of a subject developing AD may be implemented using any device capable of implementing the aforementioned described methods. Examples of devices that may be used include, but are not limited to, electronic computational devices, including computers of all types. When the methods described in this patent are implemented in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods may also be provided over an electronic network, for example, over the internet, World Wide Web, an intranet, or other network.

In one example, the methods as described herein may be implemented in a system comprising a processor and a computer readable medium that includes program code means for causing the system to carry out the steps of the methods described in this patent. The processor may be any processor capable of carrying out the operations needed for implementation of the methods. The program code means may be any code that when implemented in the system can cause the system to carry out the steps of the methods described in this patent. Examples of program code means include but are not limited to instructions to carry out the methods described in this patent written in a high level computer language such as C++, Java, or Fortran; instructions to carry out the methods described in this patent written in a low level computer language such as assembly language; or instructions to carry out the methods described in this patent in a computer executable form such as compiled and linked machine language.

Data generated by detection of relevant biomarkers can be analysed with the use of a programmable digital computer. The computer program analyses the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. Analysis generally involves the identification of peaks in the spectrum that represent signal from an analyte. Peak selection can be done visually, but software is available, as part of that can automate the detection of peaks.

### 12. Monitoring Progression of AD

In a further aspect, the invention provides methods of monitoring progression of AD in an AD patient. An individual with “Questionable AD” as used herein for quantitative data (also called absolute measurement) is an individual who (a) has been diagnosed with AD or has been given a diagnosis of probable AD, and (b) has either been assessed with the Mini-Mental State Examination (MMSE) (referenced in Folstein et al., 1975) and scored 25-28 or would achieve a MMSE score of 25-28 upon MMSE testing. Accordingly, “Questionable AD” refers to AD in an individual having scored 25-28 on the MMSE and or would achieve a MMSE score of 25-28 upon MMSE testing.

The reference level may be a predetermined level considered ‘normal’ for the particular biomarker (e.g., an average level for age-matched and/or sex-matched individuals not diagnosed with AD), or may be a historical reference level for the particular patient (e.g., a biomarker level that was obtained from a sample derived from the same individual, but at an earlier point in time). Reference levels which are determined contemporaneously (e.g., a reference value that is derived from a pool of samples including the sample being tested) are also contemplated. Accordingly, the invention provides methods for monitoring progression of AD in an AD patient by obtaining quantitative values for each biomarker in the set from a biological sample, such as for example, a biological sample and comparing measured values to reference values. For example, a decrease or increase in the measured value indicates or suggests (diagnoses or suggests a diagnosis) progression (e.g., an increase in the severity) of AD in the AD patient. In one example, the monitoring of the AD status of a subject or patent may be monitored through measurement of the values of the relevant biomarkers to determine if the AD status as ascertained by actual, predicted or theoretical SUVR scores, changes from greater than SUVR 1.5 (indicating a likely positive AD status) to less then SUVR 1.5 (indicating a normal or unlikely negative AD status). In a further example, the status of AD in a patent or subject may be monitored to determine if the AD status is made worse, such that the AD status changes from less than SUVR 1.5 (indicating a normal or unlikely negative AD status), to being greater than SUVR 1.5 (indicating a likely positive AD status). It may also be considered that an indication provided by a change in SUVR of 1.1 to 1.3 may suggest that the progression of the disease in the subject is getting worse, or alternatively a change from 1.4 to 1.3 may indicate that the subject is getter better.

The methods of the present invention further provide for the monitoring of the values of relevant biomarkers identified by the methods of the present invention and correlating these with the predicted amount of neocortical amyloid loading in a subject based on a predetermined set of relevant coefficients or a predictive model related to the identified biomarkers.

The results of the comparison between the measured value(s) and the reference value(s) are used to diagnose or aid in the diagnosis of AD, or to monitor progression of AD in an AD patient. Accordingly, if the comparison indicates a difference (that is, an increase or decrease) between the measured value(s) and the reference value(s) that is suggestive/indicative of AD, then the appropriate diagnosis is aided in or made. Conversely, if the comparison of the measured level(s) to the reference level(s) does not indicate differences that suggest or indicate a diagnosis of AD, then the appropriate diagnosis is not aided in or made.

The present invention will now be more fully described by reference to the following non-limiting Examples.

### EXAMPLES

### Example 1

### 1. Sample Collection and Analysis

### 1.1 Data Sets

Two data sets were used. The first set was taken from the Australian Imaging, Biomarkers and Lifestyle (AIBL) Study. Detailed information on the study design and enrolment procedures have been discussed by (Ellis et al. 2009). The cohort consists of 1090 subjects (207 had clinically determined Alzheimers. Disease (AD), 129 had Mild Cognitive Impairment (MCI) and 754 were healthy controls (HC) HC). 273 of the subjects had both blood measurements taken and imaging (PiB-PET). At 18 month timeframe, PET images were taken and samples taken for blood measurements, however the blood measurements have yet to be assayed.

The second set was obtained from the Alzheimer's Disease Neuroimaging Initiative (ADNI) database (http://www.loni.ucla.edu/ADNI). Each of these samples had had baseline blood measurements taken and had been PET imaged at either baseline or a twelve month follow-up. Information on the ADNI study is detailed by (Mueller et al., The Alzheimer's disease neuroimaging initiative. Neuroimaging Clinics of North America, 15, 869, 2005). Briefly, in 2003, the National Institute on Aging (NIA), the National Institute of Biomedical Imaging and Bioengineering (NIBIB), the Food and Drug Administration (FDA), private pharmaceutical companies and non-profit organizations launched ADNI and Michael W. Weiner, MD of VA Medical Center and University of California is the principal investigator.

### 1.2 Blood

80 ml of fasted blood samples were taken for 1090 of the subjects of the first cohort. Of the 80 ml blood samples: 27 ml was forwarded to a clinical pathology laboratory (Melbourne Health in Melbourne, and PathWest Laboratory Medicine WA in Perth) for baseline testing, which included full blood examination; 0.5 ml was forwarded for apolipoprotein E genotyping and 0.5 ml of whole blood was stored in liquid nitrogen.

The plasma fraction obtained from the remaining blood sample was centrifuged at 1800 g for 15 minutes at room temperature and then transferred to a polypropylene tube and stored in liquid nitrogen until time of analysis. 0.5 ml aliquots that had not been allowed any freeze-thaw cycles were shipped to Rules-Based Medicine (RBM; Austin, Tex., www.rulesbasedmedicine.com) for analysis using commercially available multiplexed luminex human discovery 151MAP panels. All assays were validated according to CLIA standards and no samples were older than 18 months at the time of analysis.

As previously described by (Lui et al. 2010), plasma Aβ was measured using both a commercial kit (INNO-BIA plasma Aβ assay, Innogenetics, Inc.) and a well-documented double sandwich ELISA technique (Mehta et al. 2000, Mehta et al. 2001, Lopez et al. 2008). Briefly, the INNO-BIA multiplex assay, based on the Luminex xMAP technique, allows for the simultaneous measurement of Aβ40 and Aβ42 (module A) or Aβ fragments (Aβn-40 and Aβn-42; module B). Both modules were run according to manufacturer instructions with the addition of an inter-plate probe wash step. Assays were read on a Luminex xMAP reader system (Bio-Plex 200 System, Bio-Rad). The ELISA utilizes, as the capture assembly, the monoclonal antibody 6E10 and two different biotinylated polyclonal antibodies for the detection of Aβ1-40 and Aβ1-42. The assay was carried out as described by (Mehta et al. 2000) and (Lopez et al. 2008) with absorbance measurements collected at 450 nm (FluoroStar, BMG).

Plasma total apoE and isoform specific (apoE4) levels were measured using a commercial assay (ApoE4/Pan ApoE ELISA, MBL co., Ltd.) described by (Gupta In Press). Briefly, the ELISA measures both total apoE and the amount of apoE4 specifically with high sensitivity using affinity purified polyclonal antibody against apoE and monoclonal antibody against apoE4. Samples were diluted 1:500 in the provided assay diluents solution prior to loading, and working concentrations of standards were prepared from dilutions of the provided calibrator, which was reconstituted 1:10 with the assay diluents solution. Diluted plasma samples or standards were loaded into microwell strips coated with anti-Human Pan-ApoE antibody and incubated for 60 min at 37° C. prior to being washed four times with wash solution. After washing, 100 μl of either a peroxidase conjugated anti-ApoE4 monoclonal antibody or a peroxidase conjugated anti-ApoE polyclonal antibody was added and incubated for 60 min at 37° C. A peroxidase substrate was then added and incubated for a further 30 min at 37° C. An acid stop solution was then added to each well to terminate the enzyme reaction prior to measurement of the optical density (O.D.) at 450 nm using a BMG microplate reader.

For the second cohort, proprietary assays in the Human DiscoveryMAP™ panel were designed and validated by Rules-Based Medicine, Inc. (RBM, Austin, Tex.), the plasma levels of a number of analytes were evaluated in UPenn specialty clinical units and centres. The resulting data was obtained from the ADNI proteomics database. Baseline clinical pathology blood measurements were also provided in the ADNI clinical database. This data covered most of the analytes necessary for validation of the findings presented here.

### 1.3 PET Imaging

Out of the 1090 subjects, for whom the blood was analysed, 273 subjects underwent PiB-PET imaging. The PiB imaging methodology is detailed by (Bourgeat et al. 2010). In brief, a Philips ADC Allegro full-ring tomograph with PIZELAR germanium oxyorthosilicate crystal detectors was utilised. The participants were injected with 375+/−18 MBq of 11C-PiB and were scanned for the post injection period of 40-60 minutes. The PiB-PET standardized uptake value (SUV) over this time period were summed over the whole cortex to give the overall neocortical SUV and then normalised with respect to the cerebellar cortex SUV, resulting in the neocortical SUV ratio (SUVR). Further details on the PiB-PET data and procedure are given by (Rowe et al. Neurobiology of Aging, 31, 1275-1283, 2010).

Based on the hypotheses that people with a high brain amyloid load will go on to develop AD and that amyloid loading is a differentiating factor of AD the imaged individuals were split into a high amyloid load group (PiB positive, neocortical SUVR score>=1.5) and a low amyloid load group (PiB negative, neocortical SUVR score<1.5), where a cut off of 1.5 is widely supported in the literature (Jack et al. Lancet Neurology, 9, 119-128, 2009), (Rowe et al. 2010). A demographic breakdown of the samples is provided in TABLE 1.

TABLE 1
the demographic breakdown of imaged participants of AIBL and the
significance of the demographic status on neocortical PiB-PET Standardized Update
Value ratio (SUVR) values on neocortical PiB-PET Standardized Update Value ratio
(SUVR) values.

| | Group/ | PiB | PiB | |
|---|---|---|---|---|
| Demographic | Measure | positive (n = 143) | negative (n = 130) | P value^{$} |
| Clinical Status | AD | 48 | 1 | |
| | MCI | 37 | 17 | |
| | HC | 58 | 112 | |
| Male | | 67 | 65 | 0.970 |
| Age (median, range) | yrs | 79 (57, 95) | 72 (62, 89) | <0.001 |
| APOE Genotype | E4/E4 | 19 | 3 | <0.001 |
| | E4/E3 | 74 | 32 | |
| | E4/E2 | 5 | 5 | |
| | E3/E3 | 40 | 67 | |
| | E3/E2 | 5 | 23 | |
| Years of Education | 0-6 | 0 | 4 | 0.720 |
| | 7-8 | 17 | 7 | |
| | 9-12 | 55 | 51 | |
| | 13-15 | 33 | 20 | |
| | 15+ | 38 | 48 | |
| BMI* (median, range) | kg/m | 25.65 (17.8, 38.5) | 25.7 (19.5, 35.6) | <0.01 |
| IPAQ^{#} | Q1 | 18 | 9 | 0.0710 |
| | Q2 | 24 | 24 | |
| | Q3 | 30 | 26 | |
| | Q4 | 26 | 42 | |
| Marital Status | Single | 7 | 3 | 0.0700 |
| | Previously | 32 | 22 | |
| | Cohabiting | | | |
| | Cohabiting | 102 | 104 | |
| Community | Involved | 97 | 90 | 0.540 |
| Involvement | | | | |

*Body Mass Index;
^{#}International Physical Activity Questionnaire Metabolic Equivalency Quartiles;
^{$}P-values obtained through standard Analysis of Variance (ANOVA) and based on ordinal neocortical SUVR values corrected for clinical diagnosis.

PET Imaging of the second cohort is described in Jagust et al. 2010. The overall neocortical SUVR scores for the purposes of validation were estimated to be the mean of the neocortex SUVR region values.

### 2. Statistical Algorithms

### 2.1 Statistical Software

Analyses were performed using R statistical software package (version 2.10, © 2009 The R Foundation for Statistical Computing). Imputations of missing data were performed using mice (multiple imputation by chain equations; version 2.4). Variable selection and prediction models were performed using the R package Random Forest (version 4.5-34). Receiver operating characteristics (ROC), sensitivities, specificities, accuracies and area under the curve (AUC) values were calculated using ROCR (version 1.0-4). Exploratory analysis of the demographic variables was achieved using the LIMMA (version 3.2.0).

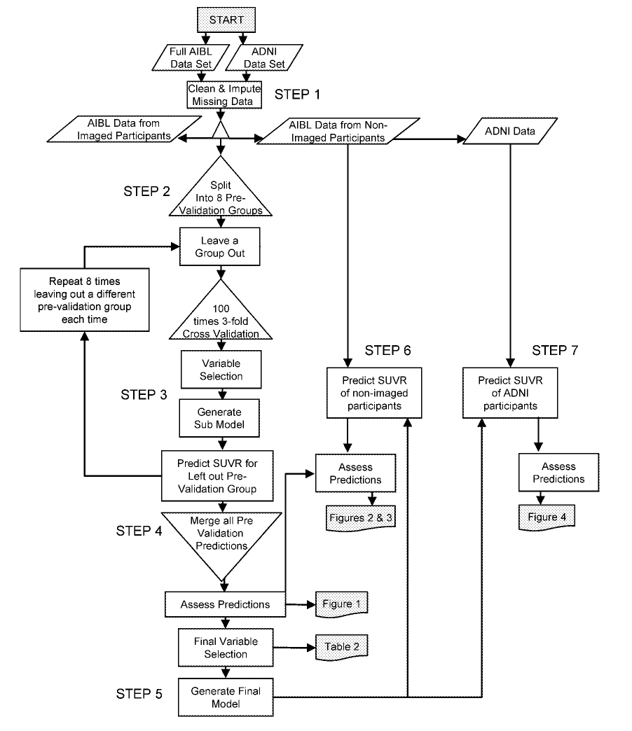

FIG. 5 outlines in brief the steps taken to develop a multivariate model that predicts the neocortical amyloid load. Whilst individual markers have previously been useful as diagnostic tools it has been found that a combination of markers provides greater sensitivity and specificity than single markers alone. For the combination to work a multivariant model such as the one described below has been developed and subsequently used as a classifier or predictor that generates SUVR scores based on PiB-PET image data. The algorithm was determined using the AIBL data some of which was segmented so that there were sets for training and cross validation (confirming that the algorithm works) sets as well as sets for which actual predictions were made.

All relevant variables, such as Amyloidβ42, Apolipoprotein E, Cortisol, BLC, IgM, IL-17, Pancreatic Polypeptide, and VCAM1 and clinical dementia, rating (CDR) sum of boxes, body mass index, were used to generate a model with applicable “relevant coefficients” to predict age-corrected neocortical SUVR scores, as measured by PiB-PET. Any of these biomarkers could be potential candidates as variable biomarker signatures. With the AIBL cohort, and confirmed with the ADNI data, a subset of 5-8 of these variables with and without the clinical markers were determined to give the sufficient sensitivity and specificity and the best accuracy.

The blood biomarker data for the 1090 AIBL participants was cleaned so that any variables with over five percent missing data, or any samples with over 50 percent missing data were removed. The data was log transformed and missing data points were imputed using multiple imputation by chain equation (AZUR et al. 2011).

TABLE 2
a panel of clinical markers identified as relevant in providing an
indication of the likelihood of an individual having AD or an AD-like
disease
Clinical Markers

Clinical Dementia
Rating (CDR) sum
of boxes
Body Mass Index

### 2.2 The Steps

[STEP 1]. Collect neocortical amyloid load data on the imaged subjects of the AIBL cohort (273 of them). This then gives a neocortical PiB-PET SUVR score. The neocortical SUVR score is a quantitative measurement of neocortical amyloid load. In general and reported widely in the literature, subjects with a neocortical SUVR score greater than 1.5 are considered to either have AD or be at risk of getting AD. Those with a neocortical SUVR score less than 1.5 are considered to be healthy and not at risk of developing AD. Thus the aim here is to predict the SUVR values (representative of amyloid load) and from the values identify subjects likely to have high neocortical amyloid loading, thus subjects who have AD or at risk or developing AD (opposed to those who don't have AD and are not at risk of getting it). This is to be achieved through using blood measures in this example, which are cheaper and easier than PiB PET imaging.

Whilst the algorithm is highly complex, for the purpose of illustrating the correlation in this example, simply, the correlation can be described as a formula such as Y=m×+c type, where m and c are the fixed (derived mathematically) coefficients “relevant coefficients” and X and Y are the variables. SUVR data can form the Y variable. Blood data “X” is also obtained for the same 273 subjects we obtain data on blood measurements (TABLE 8) and demographic and neurospyche variables (TABLE 3). It will be appreciated that the formula will not necessary be represented by this simple linear relationship, but will be a complex, multivariate possibly bimodal formula being continuous or non-continuous.

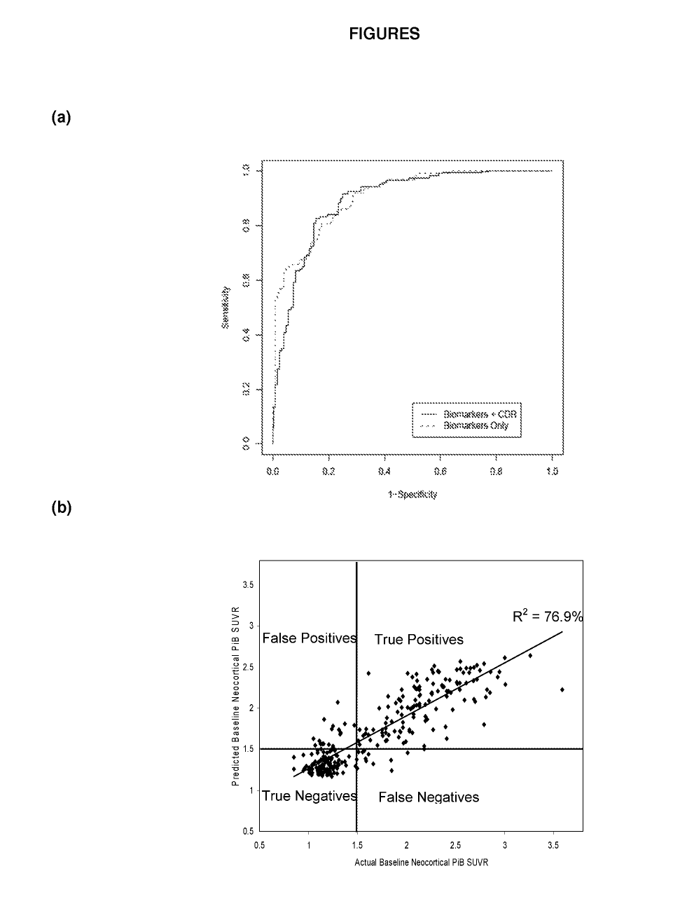

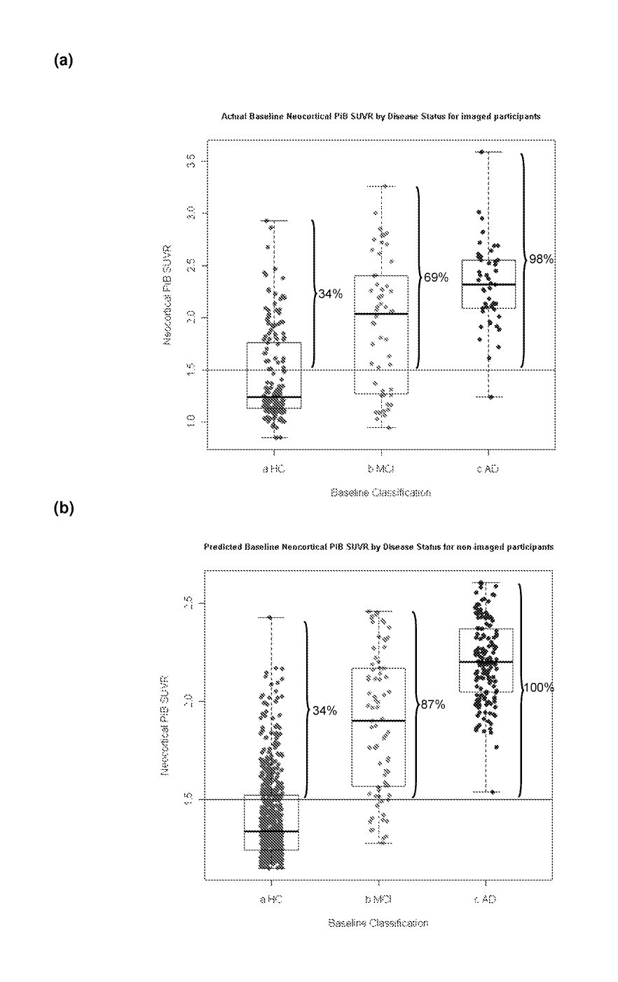

TABLE 3
a data set of other bio-, demographic and clinical markers of
interest that can be appended to the marker data set obtained
from blood data.

| | | Community | Anaemia |
|---|---|---|---|
| Gender | Location Sampled | Involvement | Status |
| Age | Marital Status | Years of Education | |
| Physical Activity | Intra Cranial | Hippocampal | |
| Quartile | Volume | Volume | |

[STEP 2] In order to provide both training sets and cross validation steps the 273 subjects are split into 8 groups and which provides cross-validation of the training set. If more data was available this spilt would not be necessary.

[STEP 3] Of the 8 groups, 7 are again randomly split into 3 groups. Two of these groups are fitted to a Random Forest model to try to predict “Y” values given the “X” values. This model is applied to the third group to predict the Y values to ascertain how well the predictions match up with the actual measured Y values. ROC/accuracy analysis is also determined to work out how well the fitted model predicted the third group. This is repeated 2 more times so that all three groups have had their Y values predicted by a model based on the other two groups. And this process is repeated 100 times. Based on the accuracy scores and importance of the X variables in these 300 Random Forest models a top panel of X variables (around 8 X variables) is picked. Another Random Forest model is fitted using only the selected X variables on the 7 groups of data. X Data from the 8^{th} set, so far put aside is then used to predict the Y values of this eighth set. This is repeated another 7 times until all subjects have a Y value prediction in this manner.

[STEP 4] All the above predictions are combined and used to generate accuracy indicators for the model—FIG. 1. Using the accuracy measures and looking at the variables chosen in each of the 8 final models a final panel of X variables is chosen.

[STEP 5] Using data from this final panel of X variables for all 273 subjects a final Random Forest model is generated which constrains fixed coefficients (is the m and c information).

[STEP 6] Finally X data for the panel of variables (the chosen biomarkers and optionally the clinical markers) for 817 AIBL subjects who hadn't been imaged are collected. That data is inputted into the final Random Forest model to get a prediction of the Y values (the PiB_PET/SUVRscores) for the non-imaged subjects. FIG. 2 demonstrates a comparison of the non-imaged subjects with the actual measured Y values of the imaged participants based on their clinical diagnosis groupings.

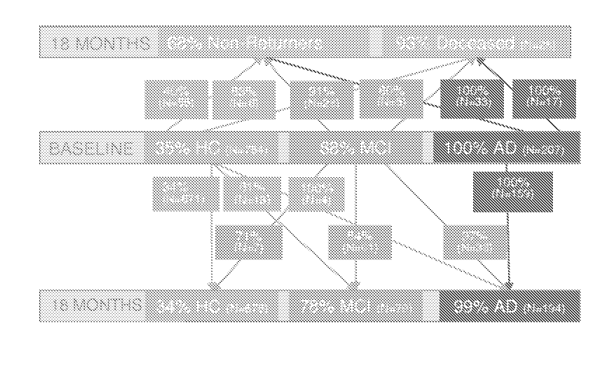

Taking the predicted Y values from the 8 models in step 4 and the predicted Y values for the non-imaged subjects in step 6, the patients can be classified as according to whether either SUVR scores (the Y values) are greater than (or equal to) 1.5 and label those subjects as being predicted to have high neocortical amyloid loading and thus at risk of getting AD, or if the SUVR score is less than 1.5 and thus predicted to have a low neocortical amyloid loading and thus less at risk. Based on the clinical diagnosis at baseline and 18-month follow up the percentage of subjects in each classification/transition group that are predicted to have high neocortical amyloid loading is then calculated, see FIG. 3.

### 3. Validation Set (The ADNI Data)

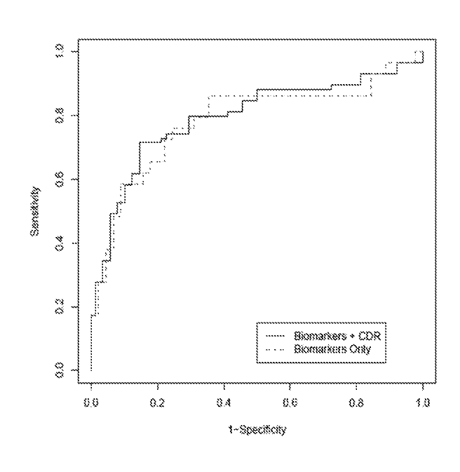

Data for 5 blood markers and the demographic and neuropsyche variables/markers) and PiB-PET score for the 74 ADNI participants was collected. The X data is inputted into the final Random Forest Model, developed in the previous example to get predicted Y values for these 74 subjects. The accuracy of these predictions compared to the actual measured values is illustrated in FIG. 4.

### 4. Results

### 4.1 Prediction of the AIBL Cohort

Firstly Step 4 lead to the receiver operating curves (ROCs) given by FIG. 1a, where for the full model sensitivity, specificity and area under the curve (AUC) of 82.5% (standard deviation (SD) of 0.891%), 84.6% (SD of 0.870%) and 89.6 (SD of 0.932), respectively, were achieved. Biomarkers without the Clinical Dementia Ratio (CDR) values from the model led to sensitivity, specificity and AUC of 80.5% (SD of 0.870%), 82.5% (SD of 1.70%) and 90.7 (SD of 1.76), respectively. The correlation of the predicted and actual (as measured by PiB-PET) neocortical SUVR is given by FIG. 1b, where good correlation with a R2 value of 76.9% is obtained.

FIG. 2 shows, for comparison, the actual neocortical SUVR for imaged participants in the ABIL cohort and the predicted neocortical SUVR for non imaged participants, AIBL cohort, partitioned over clinical diagnosis.

Secondly the predicted neocortical amyloid load was assessed against the baseline and eighteen month follow-up clinical status, refer to FIG. 3. It can be seen that: at baseline all AD, 87% MCI and 35% HC participants were deemed PiB positive; at eighteen months 99% AD, 78% MCI and 34% HC participants were deemed PiB positive; that 97% of participants transitioning to AD at eighteen months were deemed PiB positive; and 88.0% of all forward transitioning participants were deemed PiB positive.

### 4.2 Prediction of the ADNI Cohort

The neocortical SUVR levels of the ADNI participants were then predicted from the models generated and assessed against the PiB-PET imaged values. The ROCs of results are given by FIG. 4. For the full model sensitivity, specificity and area under the curve (AUC) of 74.1%, 77.3% and 79.4, respectively, were achieved. Biomarkers without the CDR from the model led to sensitivity, specificity and AUC of 75.8%, 75.6% and 78.4, respectively.

### 5. New Cohort Set

In clinical practice a patient would have blood measurements taken (possibly measuring biomarkers in TABLE 8, or any one of the RBM marker). The measurements may preferably include, along with the ones in TABLE 8, matrix metallopeptidase 2 (MMP2), AXL Receptor Tyrosine Kinase (AXL), Hepatocyte Growth Factor (HGF), Fatty Acid Synthase (FAS), glucose, Chromium isotope 52 Insulin-like Growth Factor Binding Protein (IGFBP2), Ca (Corrected)-Calcium, Serum glutanic Oxaloacetic Transaminase (SGOT), I.309 cytokine

The measurements may be made on a microchip so that a single analysis is performed. The results would then be inputted into the Random Forest model which could be embedded into a computer program. The clinician, nurse, medical administrator or general practitioner would input data such as the age off the subject

This Random Forest model will then provide a predicted y value. If this predicted y value is greater than (or equal to 1.5) the subject will be deemed to either have or be at risk of developing AD [the computer program may respond with risk=TRUE, or similar]. If this predicted Y value is less than 1.5 the subject will be deemed to not have nor be at risk of developing AD [the computer program may respond with risk=FALSE, or similar].

The computer program may also give information as to where on the scale the individual sits (either as a percentile of a measured population or as a percentile of the y value in terms of measured maximums and minimums). For example there may be 100 people in the said population and this subject has a predicted Y value that is higher than 37 of the people but lower than 63 of the people and the subject may be said to be in the 37^{th} percentile. Or the minimum recorded value may be 0.9 and the maximum recorded value may be 3.3, the subject has a predicted value of 1.3, therefore would be said to lie in the 16^{th} percentile.

If a second test for the individual is performed, the program may provide some type of change score/monitoring information. For instance at time point 1 the subject may have had a predicted Y value of 1.3 and at time point 2 the predicted Y value is 1.4. The change score is thus +0.1. [The program may respond with this or may provide a change in percentile score similar to what is discussed above—i.e. the subject was in the 37^{th} percentile but is now in the 42^{nd} percentile].

### Example 2

### 1.1 Data Sets

The same samples from the AIBL cohort and the ADNI discussed in Example 1 were used in this second round analysis.

In view of the difficulty that could be encountered by when using certain markers in a clinical setting—difficulties because of their assay measurements being potentially unreliable, tricky to perform or too variable certain markers as used in Example 1 were ignored in this second round analysis. In their place, four other bio-, demographic and clinical markers of interest were appended to the blood analytes; the markers included are Gender, ApoE ε4 carrier status, Years of Education and CDR sum of boxes.

The demographic make-up of both the imaged (AIBL and ADNI) datasets split by high and low NAB for these four markers, Age and Clinical Classification (HC, MCI or AD) are given by TABLE 4. Demographic differences between the high and low NAB were assessed using a X2 test for the categorical variables, analysis of variance (ANOVA) for the continuous variables was applied, due to the non-normality of the CDR sum of boxes the Mann-Whitney U test was applied. Also the demographic make-up of both the AIBL and ADNI imaged cohorts as well as the AIBL non-Imaged cohort split by Clinical Classification for these four markers and age are given in TABLE 6.

TABLE 4
the Demographic and Clinical make-up of the AIBL and ADNI imaged sub-
cohorts

| | | High Neocortical | Low Neocortical | |
|---|---|---|---|---|
| | | SUVR (>1.3) | SUVR (<1.3) | p-value |
| AIBL | Number of Participants: [N] | 160 | 113 | |
| Imaged | Clinical Diagnosis: [N (%)] | HC: 72 (45) | HC: 97 (86) | <0.001 |
| Cohort | | MCI: 40 (25) | MCI: 15 (13) | |
| | | AD: 48 (30) | AD: 1 (1) | |
| | Age: [mean (SD)] | 74.5 (7.7) | 69.7 (7.2) | <0.001 |
| | Gender; Males: [N (%)] | 79 (49) | 53 (47) | 0.78 |
| | Years of Education: | <9: 20 (13) | <9: 8 (7) | 0.05 |
| | [N (%)] | 9-12: 63 (39) | 9-12: 43 (38) | |
| | | 13-15: 34 (21) | 13-15: 19 (17) | |
| | | >15: 43 (27) | >15: 43 (38) | |
| | CDR Sum of Boxes: | 1.8 (2.5) | 0.2 (0.6) | <0.001 |
| | [mean (SD)] | | | |
| | APOE e4 Positive: [N (%)] | 106 (66) | 32 (28) | <0.001 |
| ADNI | Number of Participants: [N] | 59 | 23 | |
| Imaged | Clinical Diagnosis: [N (%)] | HC: 0 (0) | HC: 3 (13) | <0.05 |
| Cohort | | MCI: 42 (71) | MCI: 18 (78) | |
| | | AD: 17 (29) | AD: 2 (9) | |
| | Age: [mean (SD)] | 78.9 (8.3) | 78.4 (7.7) | 0.78 |
| | Gender; Males: [N (%)] | 20 (34) | 7 (30) | 0.97 |
| | Years of Education: | <9: 0 (0) | <9: 0 (0) | 0.15 |
| | [N (%)] | 9-12: 10 (17) | 9-12: 7 (30) | |
| | | 13-15: 11 (19) | 13-15: 1 (4) | |
| | | >15: 38 (64) | >15: 15 (65) | |
| | CDR Sum of Boxes: | 2.4 (1.7) | 1.5 (1.2) | 0.03 |
| | [mean (SD)] | | | |
| | APOE e4 Positive: [N (%)] | 56 (95) | 21 (91) | 0.92 |

### 1.2 Dataset Quality Control

The AIBL dataset, comprised of 57 pathology blood analytes and 169 plasma analytes (151 from the MyriadRBM xMap discovery panel version one, 13 Metals, APOE levels, Innogenetics and Mehta based ELISAs for Aβ₁₋₄₀ and Aβ₁₋₄₂), was cleaned so that any variables with over five percent missing data, or any samples with over 50 percent missing data were removed. This resulted in a working dataset of 176 blood analytes (53 Pathology, 111 MyriadRBM, 7 Metals, APOE levels, Innogenetics and Mehta Aβ₁₋₄₀ and Aβ₁₋₄₂; listed in TABLE 7A-E) for 1090 AIBL subjects, 273 of whom had undergone PiB-PET imaging

### 1.3 Univariate Analyses

The 176 blood analytes were also assessed, using Analysis of Covariance (ANCOVA), to see if their concentrations differed between participants with high versus low NAB. The results were corrected for age, site, gender and ApoE ε4 carrier status (by inclusion of these variables in the analysis) and to minimize any false positive results the p-values were adjusted for false discovery rate (FDR). Secondly the 176 blood analytes were assessed for correlation with the continuous SUVR variable using Multiple Linear Regression analysis (again corrected for age, site, gender, ApoE ε4 carrier status and with p-values adjusted for FDR).

### 1.4 Multivariate Analyses

The 176 blood analytes and four other markers listed above (Gender, ApoE ε4 carrier status, Years of Education and CDR sum of boxes), for the 273 imaged AIBL participants were adopted for variable selection and model generation procedures to predict age-corrected neocortical SUVR values, as measured by PiB-PET. The data from AIBL participants who had not undergone image analysis (and from all ADNI participants) was set aside at this stage.

Three algorithms were utilized for variable selection to obtain an informative panel of biomarkers: 1) Random Forest (RF) analysis was implemented to determine blood biomarkers that correlated with the continuous SUVR variable, 2) Support Vector Regression (SVR), with a radial based function (rbf) kernel coupled with a person's correlation feature selection filter, was also implemented to determine blood biomarkers that correlated with the continuous SUVR variable and 3) Support Vector Machine (SVM), with an rbf kernel coupled with signal to noise ratio feature selection, analysis was implemented to find biomarkers that were differentially expressed between high and low NAB groups.

In all multivariate analyses, three-fold cross validation with 100 repeats was used for the purposes of variable selection. For each of the three algorithms the smallest panels of variables that gave the highest performance statistics were considered. Models using the three respective variable panels were used to make NAB predictions, again using 100 times three-fold cross validation for the purposes of reporting performance statistics. For the two models developed on the continuous SUVR variable, the resultant predictions of SUVR were split into predicted high or low NAB based on a relevant cut off again for the purposes of reporting performance statistics. Sensitivities, specificities and area under the curve (AUC) performance indicators with standard deviations (SD) were calculated for each of the three panels. A final model was then generated based on all of the 273 samples for the panel of variables that gave the better performance indicators. This model was then applied to predict high or low NAB for non-imaged AIBL samples and ADNI samples. Again performance was assessed, for the ADNI validation set this was using Sensitivity, Specificity and AUC statistics. Due to there not being any actual NAB information for the non-imaged AIBL samples the percentage of those predicted to have high NAB in each of the clinical diagnosis groups was compared to that of the imaged samples and the literature.

### 1.5 Statistical Software

Analyses were performed using R statistical software package as previously described. Imputations of missing data were performed using multiple imputations by chain equations (mice) (van Buuren S, Groothuis-Oudshoorn K. mice: Multivariate Imputation by Chained Equations in R. Journal of Statistical Software 2011; 45:1-67). The univalent and multiple regression analysis used the rms (Harrell Jr F E. rms: Regression Modeling Strategies. In, 2012), car (Fox J, Sandford. An {R} Companion to Applied Regression: Sage, 2011) and MASS (Venables W N R, B. D. Modern Applied Statistics with S. In. Fourth ed: Springer, 2002) packages. Random Forest variable selection and prediction models were performed using the package Random Forest (Liaw A, Matthew. Classification and Regression by randomForest. In: R News, 2002: 18-22). SVM/R was performed with the e1071 package (Dimitriadou E, KurtLeisch, FriedrichMeyer, DavidWeingessel, Andreas. e1071: Misc Functions of the Department of Statistic. In, 2011). Receiver operating characteristics (ROC), sensitivities, specificities, accuracies and area under the curve (AUC) values were calculated using ROCR (Sing T, OliverBeerenwinkel, NikoLengauer, Thomas. ROCR: Visualizing the performance of scoring classifiers. In, 2009).

### 2. Results

### 2.1 The Demographics

TABLE 4 described the demographic make-up of the 273 imaged AIBL participants and the 82 imaged ADNI participants. It can be seen that there is an enrichment of MCI and AD subjects in the high NAB groups for both AIBL and ADNI. There was also an enrichment of elderly and ApoE ε4 carriers in the AIBL high NAB group.

### 2.2 Blood Based Analyte Differences Between High and Low NAB

TABLE 5 details the age, gender, site and ApoE ε4 carrier status adjusted means of the blood based analytes for the two NAB groups. The differences in means were assessed using ANCOVA. After adjusting for false discovery rates, five analytes showed a significant difference between the NAB groups: Immunoglobulin 1 (IgM 1) and free thyroxine (FT4) were found to be lower in the high NAB group (p=0.019 and 0.009 respectively, whilst macrophage inflammatory protein 1α (MIP1α), Pancreatic Polypeptide (PPY) and Vascular cell adhesion protein (VCAM 1) were all found to be elevated in the high NAB group (p=0.027, 0.01 and 0.01 respectively). This supports the fact that one biomarker panel could be used to determine a correlation with SUV.

TABLE 5
the ROC for the efficacy of the cross-validated
Random Forest prediction models applied to both
the AIBL and the ADNI imaged sub-cohorts

| | AIBL Imaged Cohort | | | ADNI Imaged Cohort | | |
|---|---|---|---|---|---|---|
| | Sens % | Spec % | AUC % | Sens | Spec | AUC |
| Model | (SD %) | (SD %) | (SD %) | % | % | % |
| M1: Age, ApoE | 79.6 | 82.4 | 87.6 | 78.3 | 76.3 | 84.7 |
| Genotype, BLC, | (1.3) | (1.2) | (0.7) | | | |
| IgM, IL.17, | | | | | | |
| Pancreatic Poly- | | | | | | |
| peptide, VCAM 1, | | | | | | |
| Aβ1-42, CDR | | | | | | |
| sum of boxes | | | | | | |
| M2: Age, ApoE | 79.6 | 79.4 | 83.9 | 73.9 | 74.5 | 81.7 |
| Genotype, BLC, | (1.6) | (1.4) | (1.0) | | | |
| IgM, IL.17, | | | | | | |
| Pancreatic Poly- | | | | | | |
| peptide, VCAM 1, | | | | | | |
| Aβ1-42 | | | | | | |
| M3: Age, ApoE | 71.4 | 71.5 | 78.3 | 65.2 | 62.7 | 70.2 |
| Genotype, CDR | (1.1) | (1.1) | (0.8) | | | |
| sum of boxes | | | | | | |
| M4: Age, ApoE | 66.7 | 66.7 | 70.2 | 73.9 | 62.7 | 68.6 |
| Genotype | (1.6) | (1.6) | (1.3) | | | |

### 2.3 Correlation Between the Continuous SUVR and Blood Based Analytes

The multiple regression analysis, including age, ApoE ε4 carrier status and NAB (high, low), across the blood based protein markers revealed that: within the clinical pathology data, only estimated glomerular filtration rate (eGFR) showed as positive correlation (p=0.002); within the RBM data set, EN RAGE showed a significant interaction between NAB and ApoE ε4 carrier status (p=0.003 and p=0.022, respectively), with the ε4 non-carrier high NAB showing a significant positive correlation, while the ε4 non-carrier low NAB showed a negative correlation. A similar result was observed for SGOT, with a significant interaction between NAB and ApoE ε4 carrier status (p=0.007 and p=0.019, respectively), with the ε4 non-carrier high NAB showing a significant positive correlation. No other correlations were found between the blood markers and the continuous SUVR, after adjusting for FDR.

TABLE 6
the demographics and traits from the AIBL imaged data split by Clinical
Diagnosis of Healthy Controls (HC), Mild Cognitive Impairment (MCI) and AD, from
the AND non-imaged data split by Clinical Diagnosis of Healthy Controls (HC), Mild
Cognitive Impairment (MCI) and AD and the ADNI imaged data split by Clinical
Diagnosis.

| | HC (169) | MCI (55) | AD (49) |
|---|---|---|---|
| Age [mean (sd)] | 71.7 (7.4) | 75.0 (7.7) | 72.5 (8.9) |
| Gender: Males [N (%)] | 82 (49) | 28 (51) | 22 (45) |
| Years of Education [N (%)] | <9: 14 (8) | <9: 7 (13) | <9: 7 (14) |
| | 9-12: 64 (38) | 9-12: 20 (36) | 9-12: 22 (45) |
| | 13-15: 34 (20) | 13-15: 10 (18) | 13-15: 9 (18) |
| | >15: 57 (34) | >15: 18 (33) | >15: 11 (22) |
| CDR sum of boxes [mean (sd)] | 0.0 (0.2) | 1.1 (0.8) | 4.9 (2.5) |
| APOE e4 positive [N (%)] | 73 (43) | 31 (56) | 34 (69) |

AIBL Imaged data split by Clinical Diagnosis

| | HC (584) | MCI (75) | AD (158) |
|---|---|---|---|
| Age [mean (sd)] | 69.6 (6.8) | 76.0 (7.7) | 80.0 (7.8) |
| Gender: Males [N (%)] | 236 (40) | 28 (37) | 58 (37) |
| Years of Education [N (%)] | <9: 47 (8) | <9: 15 (20) | <9: 39 (25) |
| | 9-12: 219 (38) | 9-12: 33 (45) | 9-12: 51 (34) |
| | 13-15: 115 (20) | 13-15: 11 (14) | 13-15: 27 (18) |
| | >15: 200 (34) | >15: 15 (20) | >15: 35 (23) |
| CDR sum of boxes [mean (sd)] | 0.0 (0.1) | 0.3 (0.8) | 6.0 (3.0) |
| APOE e4 positive [N (%)] | 126 (22) | 35 (47) | 95 (60) |

AIBL Non-Imaged data split by Clinical Diagnosis

| | HC (3) | MCI (60) | AD (19) |
|---|---|---|---|
| Age [mean (sd)] | 81.3 (5.5) | 79.2 (8.0) | 77.2 (8.7) |
| Gender: Males [N (%)] | 2 (67) | 18 (30) | 7 (37) |
| Years of Education [N (%)] | <9: 0 (0) | <9: 0 (0) | <9: 0 (0) |
| | 9-12: 1 (33) | 9-12: 10 (17) | 9-12: 6 (32) |
| | 13-15: 1 (33) | 13-15: 7 (12) | 13-15: 4 (21) |
| | >15: 1 (33) | >15: 43 (72) | >15: 9 (47) |
| CDR sum of boxes [mean (sd)] | 0.2 (0.3) | 1.5 (0.7) | 4.4 (1.4) |
| APOE e4 positive [N (%)] | 2 (67) | 56 (93) | 19 (100) |

ADNI Imaged data split by Clinical Diagnosis

### 2.4 Multivariate Analyses

### 2.4.1 Biomarker Identification

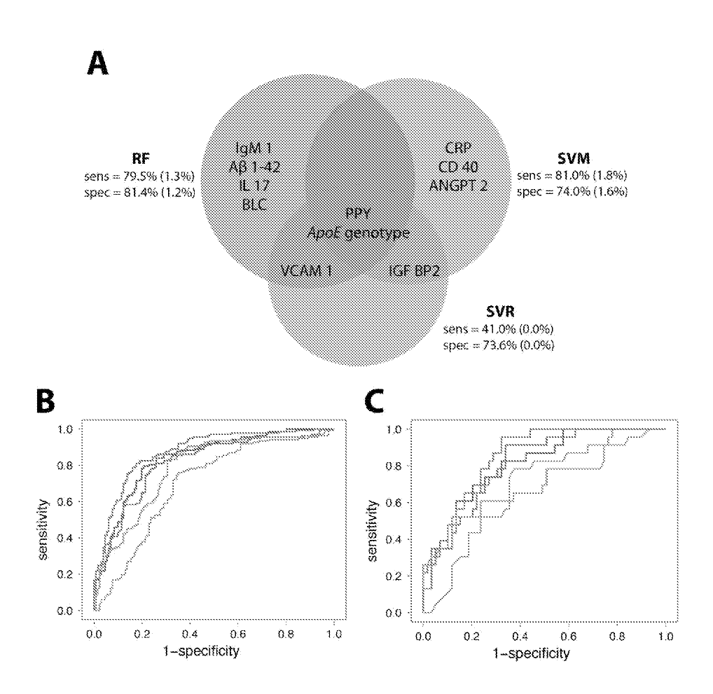

Using the three algorithms a total of eleven blood analytes were identified as being useful in predicting high versus low NAB. The RF model identified seven analytes (Amyloid Beta 1-42 (Aβ₁₋₄₂), ApoE ε4 carrier status, B-Lymphocyte Chemoattractant (BLC), IgM 1, Interleukin 17 (IL 17), PPY and VCAM 1), the SVR model identified four analytes (ApoE ε4 carrier status, Insulin-like Growth Factor-Binding Protein 2 (IGF BP 2), PPY and VCAM 1) and the SVM model identified six analytes (Angioprotein 2 (ANGPT 2), ApoE ε4 carrier status, CD40 protein (CD40), C-reactive protein (CRP), IGF BP 2, PPY). There was some overlap between the analytes being identified, with PPY and ApoE ε4 carrier status being identified by all three algorithms, see FIG. 6A. Three of these analytes (IgM 1, PPY and VCAM 1) were also identified as being significantly different between the NAB groups using ANCOVA.

### 2.4.2 Performance Statistics

The cross-validated RF model achieved 79.5% sensitivity (SD=1.3%) and 81.4% specificity (SD=1.2%), the cross-validated SVR model achieved a sensitivity and specificity of 41.0% (SD=0%) and 73.6% (SD=0%), respectively and the SVM model achieved a sensitivity and specificity of 81.0% (SD=1.8%) and 74.0% (SD=1.6%), respectively. For the best performing model, RF, four separate models were constructed to show the value added by the inclusion of the blood-based biomarkers. Model 1 (M1) blood based markers, ApoE genotype, age and CDR sum of boxes (AUC of 87.6%); model 2 (M2) blood based markers, ApoE genotype and age (AUC of 83.9%); model 3 (M3) age, ApoE genotype and CDR sum of boxes (AUC of 78.3%); model 4 (M4) age and ApoE genotype (AUC=70.2%). It can be seen that addition of the blood based markers to the models, resulted in increases in performance of 9% (M1 cf. M3) and nearly 14% (M2 cf. M4). The inclusion of the neuropsychological measure, CDR sum of boxes, improved the models by 10% (M1 cf. M2) and 4% (M3 cf. M4). Full performance statistics are given by TABLE 5 and FIG. 6 B.

### 2.4.3 Application to the ADNI Validation Samples

Unfortunately there was no measurement for IL 17 for the ADNI cohort, so the median IL 17 measurement from the AIBL cohort was substituted for each of the 82 ADNI samples. Then, the four RF models (M1: M4) generated using the AIBL samples were applied to the ADNI validation dataset for predicting high or low NAB, performance statistics are given by TABLE 5 and FIG. 6 C. It can be seen that M4 achieves 84.7% AUC when applied to the ADNI cohort.

TABLE 7 (a)
The RBM panel. The age, gender and ApoE ε4 carrier status adjusted
marginal means (sd) for the low PiB loaders (<1.3 SUVR) and the high PiB loaders
(>=1.3 SUVR) for the analytes from the standard clinical pathology panel.
The p value is the ANCOVA p-value and the adjusted (adj) p-value is adjusted for
False Discovery Rate (FDR).

| | Low NAB [mean | | High NAB | | | |
|---|---|---|---|---|---|---|
| | (SD) | | [mean (SD) | | Raw p | Adj p |
| A1AT | 2.35 | (0.79) | 2.43 | (0.82) | 0.258 | 0.698 |
| ACE CD143 | 155.84 | (103.51) | 145.16 | (96.79) | 0.236 | 0.676 |
| Adiponectin | 5.02 | (3.91) | 4.86 | (3.82) | 0.74 | 0.931 |
| Alpha 2 Macroglobulin | 0.91 | (0.24) | 0.93 | (0.24) | 0.216 | 0.652 |
| AFP | 4.08 | (3.91) | 3.44 | (3.43) | 0.035 | 0.264 |
| ANGPT 2 | 8 | (4.69) | 8.88 | (5.17) | 0.035 | 0.264 |
| Angiotensinogen | 15.66 | (35.04) | 21.44 | (47.36) | 0.08 | 0.415 |
| Apo A1 | 0.35 | (0.19) | 0.35 | (0.19) | 0.822 | 0.954 |
| Apo B | 695.2 | (373.51) | 705.99 | (380.57) | 0.683 | 0.926 |
| Apolipoprotein CIII | 96.72 | (61.28) | 93.59 | (59.52) | 0.52 | 0.841 |
| Apo D | 110.36 | (35.99) | 104.67 | (34.26) | 0.067 | 0.389 |
| Apo E | 44.95 | (26.6) | 43.75 | (25.99) | 0.549 | 0.856 |
| Apo H | 239.29 | (91.97) | 239.86 | (92.5) | 0.951 | 0.986 |
| AXL | 11.25 | (4.71) | 12.03 | (5.03) | 0.047 | 0.322 |
| B2M | 1.72 | (0.68) | 1.81 | (0.7) | 0.13 | 0.529 |
| Betacellulin | 187.85 | (87.23) | 188.3 | (87.73) | 0.985 | 0.991 |
| BLC | 22.43 | (24.11) | 25.94 | (27.81) | 0.093 | 0.45 |
| BMP 6 | 14.6 | (20.92) | 10.75 | (15.81) | 0.009 | 0.118 |
| BDNF | 1.34 | (2.15) | 1.24 | (2.07) | 0.552 | 0.856 |
| C3 | 0.85 | (0.27) | 0.86 | (0.27) | 0.631 | 0.892 |
| CD40 | 0.91 | (0.39) | 0.97 | (0.4) | 0.071 | 0.399 |
| CD40 Ligand | 0.2 | (0.29) | 0.18 | (0.29) | 0.513 | 0.84 |
| CEA | 1.63 | (1.56) | 1.66 | (1.59) | 0.836 | 0.955 |
| CgA | 511.74 | (445.45) | 513.21 | (448.25) | 0.9 | 0.975 |
| Complement Factor H | 2814.06 | (1798.43) | 2851.44 | (1828.47) | 0.772 | 0.94 |
| CK MB | 0.44 | (0.39) | 0.45 | (0.4) | 0.602 | 0.881 |
| Cortisol | 97.51 | (44.18) | 103.98 | (47.24) | 0.096 | 0.458 |
| CRP | 1.84 | (2.87) | 1.53 | (2.56) | 0.172 | 0.596 |
| EGF | 48.5 | (67.12) | 47.03 | (65.35) | 0.753 | 0.935 |
| EGF R | 128.17 | (58.33) | 121.38 | (55.45) | 0.161 | 0.596 |
| ENA 78 | 0.5 | (0.77) | 0.46 | (0.76) | 0.51 | 0.84 |
| EN RAGE | 4.44 | (5.21) | 4.64 | (5.42) | 0.67 | 0.919 |
| Eotaxin | 94.4 | (99.06) | 93.66 | (98.63) | 0.931 | 0.983 |
| Factor VII | 538.9 | (263.69) | 538.15 | (264.21) | 0.915 | 0.978 |
| FAS | 7.64 | (4.18) | 8.28 | (4.51) | 0.09 | 0.44 |
| FasL | 129.66 | (119.41) | 110.39 | (102.15) | 0.031 | 0.254 |
| Ferritin | 121.78 | (159.16) | 135.94 | (178.13) | 0.311 | 0.755 |
| Fibrinogen | 4.56 | (1.94) | 4.62 | (1.97) | 0.709 | 0.931 |
| FSH | 12.59 | (8.55) | 12.56 | (8.56) | 0.937 | 0.983 |
| G CSF | 7.21 | (6.03) | 7.45 | (6.22) | 0.612 | 0.883 |
| GH | 1.53 | (2.57) | 1.31 | (2.35) | 0.296 | 0.746 |
| GLP 1 total | 31.96 | (27.19) | 30.93 | (26.42) | 0.618 | 0.883 |
| Glucagon | 508.46 | (430.98) | 519.5 | (441.82) | 0.759 | 0.939 |
| GRO alpha | 300.62 | (304.66) | 280.84 | (285.65) | 0.384 | 0.79 |
| Haptoglobin | 1.45 | (1.34) | 1.44 | (1.33) | 0.862 | 0.957 |
| HB EGF | 101.08 | (75.31) | 90.62 | (67.82) | 0.076 | 0.41 |
| HCC 4 | 5.16 | (3.02) | 5.22 | (3.05) | 0.816 | 0.954 |
| HGF | 2.54 | (1.45) | 2.8 | (1.56) | 0.043 | 0.307 |
| I 309 | 297.47 | (592.87) | 239.19 | (478.78) | 0.168 | 0.596 |
| ICAM 1 | 135.74 | (52.79) | 132.76 | (51.82) | 0.489 | 0.831 |
| IgA | 1.39 | (1.1) | 1.43 | (1.12) | 0.663 | 0.918 |
| IgE | 30.07 | (64.22) | 30.93 | (66.23) | 0.868 | 0.957 |
| IGF BP 2 | 291.98 | (253.61) | 335.95 | (292.66) | 0.044 | 0.307 |
| IgM | 1.01 | (0.86) | 0.78 | (0.76) | 0.001 | 0.019 |
| IL 10 | 11.58 | (4.9) | 12.23 | (5.17) | 0.108 | 0.488 |
| IL 12p70 | 56.85 | (23.3) | 57.22 | (23.52) | 0.818 | 0.954 |
| IL 13 | 102.34 | (63.63) | 97.62 | (60.93) | 0.436 | 0.818 |
| IL 15 | 0.8 | (0.39) | 0.77 | (0.38) | 0.408 | 0.805 |
| IL 16 | 429.95 | (317.88) | 483.56 | (358.63) | 0.058 | 0.365 |
| IL 17 | 31.4 | (12.08) | 29.88 | (11.56) | 0.13 | 0.529 |
| IL 18 | 225.52 | (151.72) | 233.44 | (157.56) | 0.528 | 0.847 |
| IL 1ra | 110.71 | (92.31) | 109.84 | (91.91) | 0.919 | 0.978 |
| IL 3 | 0.33 | (0.26) | 0.32 | (0.26) | 0.559 | 0.861 |
| IL 4 | 59.84 | (50.45) | 60.49 | (51.16) | 0.861 | 0.957 |
| IL 5 | 7.17 | (5.81) | 7.05 | (5.75) | 0.908 | 0.978 |
| IL 8 | 10.03 | (6.4) | 9.51 | (6.12) | 0.322 | 0.756 |
| Insulin | 1.63 | (2.07) | 1.86 | (2.26) | 0.211 | 0.648 |
| Leptin | 7.78 | (11.29) | 6.98 | (10.3) | 0.368 | 0.778 |
| LH | 0.73 | (0.65) | 0.72 | (0.65) | 0.838 | 0.955 |
| Lipoprotein a | 69.71 | (147.52) | 76.05 | (161.31) | 0.639 | 0.9 |
| MCP 1 | 131.81 | (99.76) | 132.62 | (100.72) | 0.918 | 0.978 |
| M CSF | 0.73 | (0.35) | 0.72 | (0.34) | 0.546 | 0.855 |
| MDC | 392.14 | (158.87) | 377.65 | (153.53) | 0.241 | 0.682 |
| MIF | 0.37 | (0.32) | 0.4 | (0.33) | 0.173 | 0.597 |
| MIP 1alpha | 89.08 | (47.39) | 102.97 | (54.88) | 0.001 | 0.027 |
| MIP 1beta | 157.92 | (153.53) | 147.9 | (144.34) | 0.389 | 0.795 |
| MMP 2 | 2432.71 | (1455.79) | 2795.02 | (1678.17) | 0.006 | 0.093 |
| MMP 9 | 58.84 | (66.86) | 58.66 | (66.9) | 0.993 | 0.997 |
| MPO | 98.5 | (105.03) | 110.13 | (117.7) | 0.214 | 0.65 |
| Myoglobin | 16.23 | (11.07) | 17.67 | (12.03) | 0.136 | 0.54 |
| NrCAM | 2.12 | (1.21) | 2.19 | (1.24) | 0.503 | 0.836 |
| PAI 1 | 26.38 | (28.36) | 24.63 | (26.64) | 0.407 | 0.805 |
| Pancreatic | 130.09 | (115.74) | 171.3 | (152.65) | <0.001 | 0.01 |
| polypeptide | | | | | | |
| PAP | 0.26 | (0.2) | 0.26 | (0.2) | 0.819 | 0.954 |
| PAPP A | 0.04 | (0.03) | 0.04 | (0.03) | 0.247 | 0.683 |
| PARC | 28.56 | (11.33) | 29.57 | (11.76) | 0.336 | 0.756 |
| PDGF | 2271.59 | (1531.52) | 2278.69 | (1541.51) | 0.968 | 0.988 |
| PRL | 8 | (5.11) | 8.51 | (5.42) | 0.308 | 0.755 |
| PSA Free | 0.39 | (0.6) | 0.44 | (0.63) | 0.369 | 0.778 |
| RANTES | 4.31 | (5.26) | 3.69 | (4.66) | 0.116 | 0.507 |
| Resistin | 2.72 | (1.62) | 2.91 | (1.71) | 0.202 | 0.628 |
| SAP | 22.31 | (11.14) | 21.2 | (10.64) | 0.209 | 0.645 |
| SCF | 290.26 | (142.56) | 285.13 | (140.52) | 0.699 | 0.931 |
| SGOT | 14.07 | (4.47) | 14.64 | (4.65) | 0.156 | 0.588 |
| SHBG | 45.91 | (31.02) | 41.32 | (28.08) | 0.088 | 0.436 |
| SOD | 49.13 | (35.88) | 53.26 | (38.97) | 0.191 | 0.616 |
| Sortilin | 5.85 | (2.08) | 5.96 | (2.12) | 0.604 | 0.881 |
| sRAGE | 3.58 | (2.97) | 3.4 | (2.86) | 0.416 | 0.805 |
| TBG | 46.33 | (14.28) | 46.21 | (14.29) | 0.886 | 0.966 |
| TECK | 56.79 | (43.32) | 57.27 | (43.82) | 0.882 | 0.964 |
| TNC | 1236.13 | (917.11) | 1333.57 | (992.7) | 0.217 | 0.652 |
| Testosterone | 1.25 | (0.65) | 1.36 | (0.68) | 0.052 | 0.339 |
| TIMP 1 | 86.87 | (28.74) | 87.54 | (29.06) | 0.797 | 0.951 |
| TNF RII | 4.8 | (2.16) | 5.16 | (2.31) | 0.046 | 0.317 |
| Thrombopoietin | 1.99 | (1.41) | 1.87 | (1.36) | 0.294 | 0.743 |
| TRAIL R3 | 9.39 | (6.05) | 10.38 | (6.66) | 0.061 | 0.372 |
| TSH | 1.64 | (1.64) | 1.67 | (1.66) | 0.878 | 0.961 |
| THBS1 | 3185.36 | (4455.02) | 2598.24 | (3646.7) | 0.076 | 0.411 |
| VCAM 1 | 609.91 | (207.95) | 676.35 | (231.34) | <0.001 | 0.01 |
| VEGF | 470.27 | (240.51) | 495.61 | (254.29) | 0.246 | 0.683 |
| vWF | 27.96 | (26.98) | 30.55 | (29.49) | 0.242 | 0.682 |

TABLE 7 (b)
the clinical pathology panel. The age, gender and ApoE ε4 carrier
status adjusted marginal means (sd) for the low PiB loaders
(<1.3 SUVR) and the high PiB loaders (>=1.3 SUVR) for
the analytes from the standard clinical pathology panel. The p value
is the ANCOVA p-value and the adjusted (adj) p-value is adjusted
for False Discovery Rate (FDR).

| | | | |
|---|---|---|---|
| Low NAB | High NAB [mean | | adj |
| [mean (SD) | (SD) | p-value | p-value |

| | | | | | | |
|---|---|---|---|---|---|---|
| Hb | 141.32 | (16.47) | 141.24 | (16.75) | 0.949 | 0.988 |
| RCC | 4.51 | (0.62) | 4.55 | (0.63) | 0.565 | 0.843 |
| MCV | 91.63 | (6.49) | 90.89 | (6.57) | 0.201 | 0.502 |
| MCH | 31.34 | (2.56) | 31.12 | (2.59) | 0.335 | 0.653 |
| MCHC | 342.08 | (8.83) | 342.32 | (8.99) | 0.702 | 0.919 |
| ESR | 10.54 | (11.2) | 10.43 | (11.51) | 0.888 | 0.967 |
| MPV | 8.53 | (1.49) | 8.64 | (1.56) | 0.381 | 0.686 |
| WCC | 5.68 | (2.1) | 5.69 | (2.14) | 0.958 | 0.991 |
| Neut | 3.34 | (1.64) | 3.48 | (1.73) | 0.361 | 0.674 |
| Lymp | 1.54 | (0.83) | 1.44 | (0.78) | 0.153 | 0.435 |
| Mono | 0.45 | (0.22) | 0.43 | (0.2) | 0.22 | 0.517 |
| Eos | 0.15 | (0.16) | 0.16 | (0.17) | 0.826 | 0.962 |
| HCY | 8.59 | (3.9) | 8.97 | (4.32) | 0.322 | 0.643 |
| B12 | 289.02 | (199.68) | 275.52 | (190.09) | 0.447 | 0.755 |
| sFol | 30.28 | (20.22) | 27.83 | (19.83) | 0.165 | 0.45 |
| rFol | 996.71 | (583.2) | 956.75 | (581.52) | 0.445 | 0.755 |
| K | 4.26 | (0.48) | 4.22 | (0.49) | 0.365 | 0.674 |
| HCO3 | 28.55 | (3.61) | 28.87 | (3.72) | 0.316 | 0.643 |
| Urea | 0.42 | (0.08) | 0.41 | (0.08) | 0.085 | 0.318 |
| Crea | 80.73 | (20.79) | 83.45 | (21.98) | 0.143 | 0.416 |
| eGFR | 66.98 | (17.12) | 65.13 | (17.27) | 0.231 | 0.518 |
| G | 5.06 | (1.01) | 5.08 | (1.04) | 0.855 | 0.962 |
| tPr | 72.81 | (7.54) | 72.99 | (7.69) | 0.773 | 0.944 |
| Alb | 42.88 | (4.2) | 43.28 | (4.34) | 0.284 | 0.6 |
| Glob | 28.63 | (5.08) | 28.41 | (6.43) | 0.986 | 0.997 |
| Bilirubin | 11.42 | (5.76) | 12.02 | (6.32) | 0.279 | 0.598 |
| ALT | 20.8 | (11.47) | 21.37 | (12.15) | 0.596 | 0.862 |
| AP | 4.39 | (0.48) | 4.35 | (0.49) | 0.362 | 0.674 |
| GGT | 22.17 | (17.33) | 21.64 | (17.01) | 0.693 | 0.919 |
| Ca corr | 2.29 | (0.13) | 2.3 | (0.14) | 0.399 | 0.705 |
| Fe | 17.65 | (8.15) | 17.81 | (8.33) | 0.801 | 0.953 |
| transferrin | 32.95 | (7.02) | 32.94 | (7.15) | 0.958 | 0.991 |
| tr.sat | 27 | (13.72) | 27.11 | (14.01) | 0.903 | 0.969 |
| Ferr | 118.3 | (140.57) | 126.87 | (153.6) | 0.479 | 0.787 |
| Ins | 5.6 | (6.63) | 6.95 | (8.42) | 0.032 | 0.169 |
| PSA | 1.23 | (1.82) | 1.57 | (2.38) | 0.222 | 0.517 |
| TNtsk!td | 3.3 | (3.51) | 3.23 | (3.52) | 0.785 | 0.95 |
| Oestradiol | 92.41 | (85.7) | 83.53 | (79.28) | 0.222 | 0.517 |
| LH | 3.57 | (1.57) | 3.51 | (1.61) | 0.72 | 0.931 |
| FT4 | 14.1 | (3.08) | 13.25 | (2.78) | 0.001 | 0.009 |
| TSH | 1.68 | (1.74) | 1.88 | (1.88) | 0.211 | 0.512 |
| FT3 | 4.64 | (0.99) | 4.43 | (0.96) | 0.015 | 0.086 |
| Chol | 5.6 | (1.68) | 5.51 | (1.72) | 0.554 | 0.84 |
| Trig | 1.14 | (0.71) | 1.15 | (0.74) | 0.91 | 0.974 |
| HDL | 1.64 | (0.65) | 1.62 | (0.66) | 0.896 | 0.967 |
| LDL | 3.23 | (1.52) | 3.17 | (1.54) | 0.656 | 0.914 |
| Ast | 23.32 | (11.14) | 23.77 | (11.67) | 0.677 | 0.919 |
| PCV | 0.41 | (0.05) | 0.41 | (0.05) | 0.884 | 0.967 |

TABLE 7 (c)
the metals panel. The age, gender and ApoE ε4 carrier status adjusted
marginal means (sd) for the low PiB loaders (<1.3 SUVR) and the high
PiB loaders (>=1.3 SUVR) for the analytes from metalomic panel.
The p value is the ANCOVA p-value and the adjusted (adj) p-value is
adjusted for False Discovery Rate (FDR).

| | Low NAB [mean | | High NAB [mean | | p- | adj p- |
|---|---|---|---|---|---|---|
| | (SD) | | (SD) | | value | value |
| Chromium 52 | 1.36 | (798.6193) | 1.35 | (793.3555) | 0.842 | 0.904 |
| Chromium 53 | 0.54 | (0.1553) | 0.57 | (0.1592) | 0.025 | 0.133 |
| Copper 65 | 15.71 | (4.2987) | 15.11 | (4.1624) | 0.091 | 0.337 |
| Iron 57 | 21.17 | (8.6145) | 21.52 | (8.7881) | 0.656 | 0.82 |
| Rubidium 85 | 2.46 | (0.7454) | 2.32 | (0.7177) | 0.023 | 0.131 |
| Selenium 78 | 4.11 | (1.2588) | 3.99 | (1.2354) | 0.256 | 0.555 |
| Zinc 66 | −0.67 | (26.147) | −0.68 | (26.1922) | 0.654 | 0.82 |

TABLE 7 (d)
plasma Aβ measurements. The age, gender and ApoE ε4 carrier
status adjusted marginal means (sd) for the low PiB loaders
(<1.3 SUVR) and the high PiB loades (>=1.3 SUVR) of the
Aβ 40, 42 measurements from the Innogentics plateform (Inno)
and the Mehta sandwhich ELISA (Mehta). The p is the ANCOVA p-
value and the adjusted p (adj p) is p adjusted for False Discovery
Rate (FDR).

| | Low NAB [mean | | High NAB [mean | | | |
|---|---|---|---|---|---|---|
| | (SD) | | (SD) | | p | adj p |
| Inno Aβ 40 | 147.89 | (56.22) | 147.03 | (56.47) | 0.854 | 0.945 |
| Inno Aβ 42 | 31.67 | (16.54) | 30.5 | (16.31) | 0.46 | 0.761 |
| Inno Aβ 40/42 ratio | 0.23 | (0.13) | 0.21 | (0.13) | 0.25 | 0.501 |
| Metha Aβ40 | 135.76 | (96.62) | 121.24 | (91.77) | 0.063 | 0.298 |
| Metha Aβ 42 | 46.06 | (24.59) | 42.67 | (22.04) | 0.091 | 0.302 |
| Methta Aβ 40/42 | 0.37 | (0.17) | 0.38 | (0.18) | 0.514 | 0.787 |
| ratio | | | | | | |

TABLE 7 (e)
plasma ApoE as measured by ELISA. The age, gender and ApoE ε4
carrier status adjusted marginal means (sd) for the low PiB loaders
(<1.3 SUVR) and the high PiB loaders (>=1.3 SUVR).

| | Low NAB | High NAB | |
|---|---|---|---|
| | [mean (SD) | [mean (SD) | p |
| ApoE | 14.88 (3.96) | 14.53 (3.97) | 0.30711 |

TABLE 8
a list of possible biomarkers, including peptides, polypeptides,
proteins, oligonucleotides, fragments thereof, and/or other
markers, such as metals, metabolites or vitamins and the like,
that could be used in providing an indication of the likelihood
of an individual having AD or an AD-like disease. Those with
an asterisk relate to Rules Based Medicine (RBM) measurements.

6Ckine
ABeta 42* (AB42)
Adiponectin*
Agouti-Related Protein
Aldose Reductase
Alpha.2.Macroglobulin*
Alpha-1-Antichymotrypsin
Alpha-1-Antitrypsin-A1AT*
Alpha-1-Microglobulin
Alpha-2-Macroglobulin
alpha1 acid glycoprotein
alanine transaminase-ALT*
albumin-Alb
alkaline phosphatase-AP*
alpha syncline
Alpha-Fetoprotein-AFP*
Amphiregulin
Angiogenin
Antithrombin 3-AT3
Angiopoietin-2-ANGPT.2*
Angiotensin-Converting Enzyme-
ACE..CD143.*
Angiotensinogen*
Annexin A1
ApoE_ECU*
Apolipoprotein AII + A285 Dimer
Apolipoprotein B-Apo.B*
Apolipoprotein C-I
Apolipoprotein D-Apo.D*
Apolipoprotein E-Apo.E*
Apolipoprotein H-Apo.H*
Apolipoprotein(a)
Apolipoprotein.CIII*
Ast
AXL Receptor Tyrosine Kinase- [symbol]
B cell-activating factor
B Lymphocyte Chemoattractant
BLC*
B12*
Baso
Bcl-2-like protein 2
Beta-2-Microglobulin-B2M*
Betacellulin*
Bilirubin*
Bone Morphogenetic Protein 6-
BMP.6*
Brain-Derived Neurotrophic Fac [symbol]
BDNF*
C3*
Caer*
Calbindin
Calcitonin
Cancer Antigen 125
Cancer Antigen 15-3
Cancer Antigen 19-9
Cancer Antigen 72-4
Carcinoembryonic Antigen-CEA*
Cathepsin D
CD 40 antigen-CD40*
CD40.Ligand*
CD5 Antigen-like
ceurolplasmin
CgA*
chemokine (C-X-C motif)*
Chemokine CC-4-CK.MB
Chromogranin-A
Ciliary Neurotrophic Factor
Cl*
Clusterin
Collagen IV
Complement C3
Complement.Factor.H*
Connective Tissue Growth Factor
Cortisol*
C-Peptide
C-Reactive Protein-CRP*
Creatine Kinase-MB
chromogranin B
Endoglin
Endostatin
Endothelin-1
Eos
Eotaxin (all subunits)*
Epidermal Growth Factor-EGF*
Epidermal Growth Factor Receptor-
EGF.R
Epiregulin
Epithelial cell adhesion molecule
Erythropoietin
E-Selectin
extracellular newly identified RAGE-
binding protein-EN.RAGE*
Ezrin
erythrocyte sedimentation rate-
ESR*
estimated glomerular
filtration rate-eGFR*
Factor.VII*
FAS*
FASLG Receptor
Family with sequence similarity 3,
member C (FAM3C (I))
Fatty Acid-Binding Protein
Ferritin*
Fetuin-A
Fibrinogen*
Fibroblast Growth
Factor 4
Fibroblast Growth Factor basic
Fibulin-1C
Follicle-Stimulating Hormone-FSH*
FT3*
G*
Galectin-3
Gelsolin
gamma glutamyl transpeptidase-
GGT*
Glucagon*
Glucagon-like Peptide 1, total-
GLP.1.total*
Glucose-6-phosphate
Isomerase
Glutamate-Cysteine Ligase
Regulatory subunit
Glutathione S-Transferase alpha
Glutathione S-Transferase Mu 1
Granulocyte Colony-Stimulating
Factor-G.CSF*
GRO.alpha*
Growth Hormone-GH*
Haptoglobin*
HCC.4*
HCY*
HE4
Heat Shock Protein 60
Heparin-Binding EGF-Like Growth
Factor-HB.EGF*
Hepatocyte Growth Factor-HGF
Hepatocyte Growth
Factor receptor
Hepsin
hemopexin
Human Chorionic
Gonadotropin beta
Human Epidermal Growth Fact [symbol]
Receptor 2
Hemoglobin-Hb*
high density liporprotein-HDL*
iron-Fe*
Immunoglobulin A-IgA*
Immunoglobulin E-IgE*
Immunoglobulin M-IgM*
Inno_AB_ratio*
Inno_AB40*
Inno_AB42*
Insulin*
Insulin-like Growth Factor Bindi [symbol]
Protein 4
Insulin-like Growth Factor Bindi [symbol]
Protein 5
Insulin-like Growth Factor Bindi [symbol]
Protein 6
Insulin-like Growth Factor-Bindi [symbol]
Protein 1
Insulin-like Growth
Factor-Binding Protein 2
Insulin-like Growth
Factor-Binding Protein
2-IGF.BP.2*
Insulin-like Growth Factor-Bindi [symbol]
Protein 3
Intercellular Adhesion Molecule
ICAM.1*
Interferon gamma
Interferon gamma Induced Prot [symbol]
10
Interferon-inducible T-cell alpha
chemoattractant
Interleukin-1 alpha
Interleukin-1 beta
Interleukin-1 receptor antagonist
Interleukin-1 receptor antagonist-
IL.1ra*
Interleukin-10
Interleukin-10-IL.10*
Interleukin-12 Subunit p40
Interleukin-12 Subunit p70
Interleukin-12 Subunit
p70 IL.12p70*
Interleukin-13-IL.13*
Interleukin-15-IL.15*
Interleukin-16-IL.16*
Interleukin-17-IL.17*
Interleukin-18-IL.18*
Interleukin-2
Interleukin-2 receptor alpha
Interleukin-25
Interleukin-3-IL.3*
Interleukin-4-IL.4*
Interleukin-5-IL.5*
Interleukin-6 receptor
Interleukin-7
Interleukin-8-IL.8*
Kallikrein 5
Kallikrein-7
Kidney Injury Molecule-1
Lactoylglutathione lyase
Latency-Associated
Long-chain plasma
ceramides C22:0
Long-chain plasma
ceramides C24:0
Peptide of Transforming
Growth Factor beta 1
Lectin-Like Oxidized LDL Receptor 1
Leptin*
Lipoprotein.a.*
Luteinizing Hormone-
LH*
Lymphotactine-Lymp*
low density lipoprotein-LDL*
Macrophage Colony-
Stimulating Factor 1-
M.CSF*
Macrophage inflammatory protein
3 beta
Macrophage Inflammatory Protein-
1 alpha
Macrophage [symbol]
flammatory Protein-1
beta
Macrophage Inflammatory Protein-
3 alpha
Macrophage Migration Inhibitory
Factor
Macrophage-Derived Chemokine-
MDC*
Macrophage-
Stimulating Protein
Malondialdehyde-Modified Low-
Density Lipoprotein
Maspin
Matrix Metalloproteinase-1
Matrix Metalloproteinase-10
Matrix
Metalloproteinase-2-
MMP.2*
Matrix Metalloproteinase-3
Matrix Metalloproteinase-7
Matrix
Metalloproteinase-9-
MMP.9*
mean corpuscular hemoglobin
concentration-MCHC*
mean platelet volume-MPV*
melanin concentrating hormone
MCH*
membrane cofactor protein 1-
MCP.1*
modified citrullinated vimentin-
MCV*
Mehta_AB_ratio*
Mehta_AB40*
Mehta_AB42*
Mesothelin
Metals.Mean.Chromium.isotope
Metals.Mean.Chromium.isotope
Metals.Mean.Copper.isotope.6
Metals.Mean.Iron.isotope.57*
Metals.Mean.Rubidium.isotope. [symbol]
Metals.Mean.Selenium.isotope. [symbol]
Metals.Mean.Zinc.isotope.66*
MHC class I chain-related protei [symbol]
Microalbumin
MIP.1 beta*
MIP.1alpha*
Mono*
Monocyte Chemotactic Protein
Monocyte Chemotactic
Protein 2
Monocyte Chemotactic Protein 3
Monocyte Chemotactic Protein 4
Monokine Induced by Gamma
Interferon
MPO*
Myoglobin*
Myeloid Progenitor Inhibitory
Factor 1-MIF*
Myeloperoxidase
neutrophil-activating peptide-
ENA.78*
Nerve Growth Factor
beta
Neuronal Cell Adhesion Molecule-
NrCAM*
Neuron-Specific Enolase
Neuropilin-1
Neutrophil Gelatinase-
Associated Lipocalin
Neutrophils*
N-terminal prohormone of brain
natriuretic peptide
Nucleoside diphosphate kinase B
Oestradiol*
Osteopontin
Osteoprotegerin
Parkinson protein 5
Parkinson protein 7
Platelet count-Plt*
potassium-K*
PPY-
Pancreatic.polypeptide*
Packed cell volume-PCV
PAI.1*
Pancreatic ribonuclease
PARC*
Pepsinogen I
Peptide YY
Peroxiredoxin-4
Phosphoserine Aminotransferase
Placenta Growth Factor
Plasminogen Activator
Inhibitor 1
Platelet-Derived Growth Factor BB-
PDGF*
Pregnancy-Associated
Plasma Protein A-
PAPP.A*
PRL*
Progesterone
Proinsulin, Intact
Proinsulin, Total
Prolactin
Prostasin
Protein I-309*
Prostate-Specific
Antigen, Free-
.PSA..Free.*
prostaglandin D synthase
Prostatic Acid Phosphatase-PAP*
Protein S100-A4
Protein S100-A6
Pulmonary and Activation-
Regulated Chemokine
Receptor tyrosine-protein kinase
erbB-3
Resistin*
red blood cell distribution width-
RDW*
Red Cell count-RCC*
red cell folate-rFol*
Saposin A
Saposin B
Saposin D
saturated transferrin-tr.sat*
serum folate-sFol*
sodium-Na*
stem cell factor-SCF*
S100 calcium-binding
protein B
Secretin
Serotransferrin
secretoneurin
Serum Amyloid P-Component- [symbol]
Serum Glutamic
Oxaloacetic
Transaminase-SGOT*
Sex Hormone-Binding Globulin-
SHBG*
Sortilin*
Squamous Cell Carcinoma Antig [symbol]
sRAGE*
Stromal cell-derived
factor-1
Superoxide Dismutase 1, solubl [symbol]
SOD*
T Lymphocyte-Secreted
Tamm-Horsfall Urinary
Glycoprotein
T-Cell-Specific Protein RANTES-
RANTES*
TECK*
Tenascin-C
Testosterone, Total
Tetranectin
Thrombomodulin
Thrombopoietin*
thymosin beta
Thrombospondin-1
Thyroglobulin
Thyroid-Stimulating Hormone-TSH*
Thyroxine-Binding Globulin-TBG*
Tissue Factor
Tissue Inhibitor of
Metalloproteinases 1-TIMP.1*
Tissue type Plasminogen activator
testosterone-Testo*
Thrombospondin 1-THBS1*
Thyroxine FT4*
total calcium Ca.total*
total protein-tPr*
TNC
TNF-Related Apoptosis-Inducing
Ligand Receptor 3
TRAIL.R3*
transferrin*
Transforming Growth Factor alpha
Transforming Growth Factor beta-3
Transthyretin
Trefoil Factor 3
Trig* [symbol]
umor Necrosis Factor alpha
Tumor Necrosis Factor beta
Tumor necrosis factor
receptor 2-TNF.RII*
Tumor Necrosis Factor Receptor I
Tyrosine kinase with Ig and EGF
homology domains 2
Urea*
Urokinase-type Plasminogen
Activator
Urokinase-type plasminogen
activator receptor
ubiquitin 3
ubiquitin 4
Vascular Cell Adhesion Molecule-1
Vascular Endothelial Growth Factor
Vascular endothelial
growth factor B
Vascular Endothelial Growth Fa [symbol] C
Vascular endothelial growth fac [symbol] D
Vascular Endothelial Growth Fa [symbol]
Receptor 1
Vascular Endothelial
Growth Factor Receptor 2
Vascular endothelial growth fac [symbol]
receptor 3
Vitamin K-Dependent Protein S
Vitronectin
von Willebrand Factor
YKL-40
Vascular cell adhesion protein 1-
VCAM.1*
Vascular endothelial growth fact [symbol]
VEGF*
von Willebrand factor-vWF*
white cell count-WCC* [symbol]
indicates data missing or illegible when filed

### 2.4.4 Application to the Non-Imaged AIBL Samples

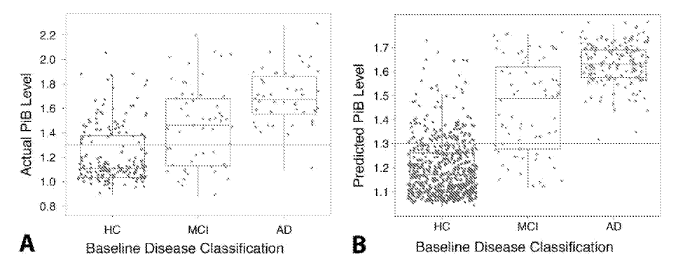

The full RF model (M4) was applied to the 817 AIBL participants who had not undergone the imaging protocol, to predict their expected NAB. All AD, 87% of MCI and 35% of HC participants were predicted to have high NAB which is comparable to the 98% AD, 69% MCI and 34% HC deemed to have high NAB for the AIBL imaged cohort through imaging protocol (FIG. 7).

### 5. Conclusions

The further list of biomarkers, namely: age, Aβ₁₋₄₂, ApoE genotype, BLC, IgM 1, IL 17, PPY and VCAM 1 were ascertained under the analysis described above. The inclusion of a clinical based cognitive score, CDR sum of boxes, slightly improves the sensitivity and specificity. Sensitivity and specificity of 79.6% and 82.4% respectively were achieved for assessing high NAB versus low NAB. When this model was applied to the ADNI cohort, reasonable predictions were made, sensitivity and specificity of 78.3% and 76.3%, respectively. This model was then also applied over the non-imaged AIBL participants to predicted percentages of individuals with high NAB for individual clinical diagnosis groups (FIG. 7). The model predicted 100% of AD, 60-75% of MCI and 20-35% of HCs have high NAB which is comparable with literature prediction percentages.

The use of the support vector machine model showed similar, slightly lower performance statistics, using different set of biomarkers (age, ApoE genotype, ANGPT-2, CD40, CRP, PPY and IGF-BP2). The only overlapping biomarkers between the Random Forest and SVM models were age, ApoE genotype, and PPY.

The only common blood based biomarker of the Random Forest and the SVM model was pancreatic polypeptide (PPY). While PPY levels are positively correlated with age, here a significant increase in the high NAB group, after adjusting for age, was seen.

While previous reported blood-based measures have shown good efficacy in discriminating between AD sufferers and aged matched controls, the results presented here identified blood-based measures, which are able to estimate the level of NAB in an individual with high accuracy. As NAB represents the amyloidal load in the brain, whose accumulation is believed to be an early event in the cascade of AD, this panel may provide much earlier blood-based disease identification.

Given the widely supported hypothesis that NAB is a predictor for progression to AD and that the findings presented here can be validated and formatted to a suitable medium for widespread use, it follows that this work may be the first step in developing an economical screen for early detection of individuals at risk of developing AD, allowing optimum treatment and intervention strategies to be employed. Such a test could also, in-turn, justify for further confirmatory tests such as PET imaging or CSF measurements.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

### REFERENCES

Agrawal et al., 1993 “Mining association rules between sets of items in large databases.” In Proc. of the ACM SIGMOD Conference on Management of Data, pages 207-216, Washington, D.C., May 1993.
Dubois, B., H. H. Feldman, C. Jacova, J. L. Cummings, S. T. DeKosky, P. Barberger-Gateau, A. Delacourte, G. Frisoni, N. C. Fox, D. Galasko, S. Gauthier, H. Hampel, G. A. Jicha, K.
Fodero-Tavoletti M T, Rowe C C, et al. Characterization of PiB Binding to White Matter in Alzheimer Disease and Other Dementias. J Nucl Med 2009; 50(2):198-204.
Folstein et al., J. Psychiatr. Res 1975; 12:1289-198
Gottardo, Statistical analysis of microarray data, A Bayesian approach. Biostatistics (2001), 11, pp 1-37
Hampel, H., K. Buerger, S. J. Teipel, A. L. W. Bokde, H. Zetterberg & K. Blennow (2008) Core candidate neurochemical and imaging biomarkers of Alzheimer's disease. Alzheimers & Dementia, 4, 38-48.
Hampel, H., R. Frank, K. Broich, S. J. Teipel, R. G. Katz, J. Hardy, K. Herholz, A. L. W. Bokde, F. Jessen, Y. C. Hoessler, W. R. Sanhai, H. Zetterberg, J. Woodcock & K. Blennow (2010) Biomarkers for Alzheimer's disease: academic, industry and regulatory perspectives. Nature Reviews Drug Discovery, 9, 560-574.
Hastie et al., Genome Biology 2001, 2: research 0003.1-0003.12
Jack, C. R., D. S. Knopman, W. J. Jagust, L. M. Shaw, P. S. Aisen, M. W. Weiner, R. C. Petersen & J. Q. Trojanowski (2010) Hypothetical model of dynamic biomarkers of the Alzheimer's pathological cascade. Lancet Neurology, 9, 119-128.
Kemppainen, N. M., S. Aalto, I. A. Wilson, K. Nagren, S. Helin, A. Bruck, V. Oikonen, M. Kailajarvi, M. Scheinin, M. Viitanen, R. Parkkola & J. O. Rinne (2006) Voxel-based analysis of PET amyloid ligand C-11 PiB uptake in Alzheimer disease. Neurology, 67, 1575-1580.
Klunk, W. E., H. Engler, A. Nordberg, Y. M. Wang, G. Blomqvist, D. P. Holt, M. Bergstrom, I. Savitcheva, G. F. Huang, S. Estrada, B. Ausen, M. L. Debnath, J. Barletta, J. C. Price, J. Sandell, B. J. Lopresti, A. Wall, P. Koivisto, G. Antoni, C. A. Mathis & B. Langstrom (2004) Imaging brain amyloid in Alzheimer's disease with Pittsburgh Compound-B. Annals of Neurology, 55, 306-319.
Klunk W E, Wang Y, et al. The binding of 2-(4′-methylaminophenyl)benzothiazole to postmortem brain homogenates is dominated by the amyloid component. J Neurosci 2003; 23(6):2086-92.
Kohonen, 1982b, Biological Cybernetics 43(1):59-69.
Liaw, A. and Wiener, M (2002) Classification and Regression by random Forest, R News, 2(3), pp 18-22.
Mueller, S. G., M. W. Weiner, L. J. Thal, R. C. Petersen, C. Jack, W. Jagust, J. Q. Trojanowski, A. W. Toga & L. Beckett (2005) The Alzheimer's disease neuroimaging initiative. Neuroimaging Clinics of North America, 15, 869
Ng, S., V. L. Villemagne, S. Berlangieri, S. T. Lee, M. Cherk, S. J. Gong, U. Ackermann, T. Saunder, H. Tochon-Danguy, G. Jones, C. Smith, G. O'Keefe, C. L. Masters & C. C. Rowe (2007) Visual assessment versus quantitative assessment of C-11-PiB PET and F-18-FDG PET for detection of Alzheimer's disease. Journal of Nuclear Medicine, 48, 547-552.
O'Bryant, S. E., G. Xiao, R. Barber, J. Reisch, R. Doody, T. Fairchild, P. Adams, S. Waring, R. Diaz-Arrastia & C. Texas Alzheimer's Res (2010) A Serum Protein-Based Algorithm
Ray, S., M. Britschgi, C. Herbert, Y. Takeda-Uchimura, A. Boxer, K. Blennow, L. F. Friedman, D. R. Galasko, M. Jutel, A. Karydas, J. A. Kaye, J. Leszek, B. L. Miller, L. Minthon, J. F. Quinn, G. D. Rabinovici, W. H. Robinson, M. N. Sabbagh, Y. T. So, D. L. Sparks, M. Tabaton, J. Tinklenberg, J. A. Yesavage, R. Tibshirani & T. Wyss-Coray (2007) Classification and prediction of clinical Alzheimer's diagnosis based on plasma signaling proteins. Nature Medicine, 13, 1359-1362.
Rowe, C. (2008) A beta deposits in older non-demented individuals with cognitive decline are indicative of preclinical Alzheimer's disease. Neuropsychologia, 46, 1688-1697.
Rowe, C. C., K. A. Ellis, M. Rimajova, P. Bourgeat, K. E. Pike, G. Jones, J. Fripp, H. Tochon-Danguy, L. Morandeau, G. O'Keefe, R. Price, P. Raniga, P. Robins, O. Acosta, N. Lenzo, C. Szoeke, O. Salvado, R. Head, R. Martins, C. L. Masters, D. Ames & V. L. Villemagne (2010) Amyloid imaging results from the Australian Imaging, Biomarkers and Lifestyle (AIBL) study of aging. Neurobiology of Aging, 31, 1275-1283.
Shaw, L. M., M. Korecka, C. M. Clark, V. M. Y. Lee & J. Q. Trojanowski (2007) Biomarkers of neurodegeneration for diagnosis and monitoring therapeutics. Nature Reviews Drug Discovery, 6, 295-303.
Tusher et al., 2001, Proc. Natl. Acad. Sci. U.S.A. 98(9):5116-21
Villemagne, V. L., K. E. Pike, D. Darby, P. Maruff, G. Savage, S. Ng, U. Ackermann, T. F. Cowie, J. Currie, S. G. Chan, G. Jones, H. Tochon-Danguy, G. O'Keefe, C. L. Masters & C.
Zweig, M. H. and Campbell, G. (1993) Receiver-Operating Characteristic (ROC) Plots: A Fundamental Evaluation Tool in Clinical Medicine, Clin. Chem. 39(4), pp 561-577.

1. A method for generating a set of relevant coefficients for predicting neocortical amyloid beta levels, comprising:
a) applying a classification algorithm to a plurality of biomarker values from a plurality of predetermined validated samples; and
b) applying the classification algorithm to a plurality of amyloid beta levels obtained from the same plurality of predetermined validated samples of step a);
wherein applying the classification algorithm generates a set of relevant coefficients capable of predicting the amyloid beta levels by correlating biomarkers to amyloid beta levels.

2. The method of claim 1, wherein the classification algorithm is simultaneously applied to the plurality of biomarker values of step a) and the plurality of amyloid beta levels from step b).

3. The method according to claim 1, wherein the classification algorithm is selected from the group consisting of Random Forests, Variable Importance Measures, Linear Discriminant Analysis (LDA), Diagonal Linear Discriminant Analysis (DLDA), Diagonal Quadratic Discriminant Analysis (DQDA), Support Vector Machines (SVM), Support Vector Regression (SVR) Neural Network, Analysis of Covariance (ANCOVA) and k-Nearest Neighbour method.

4. The method according to claim 1, wherein the plurality of predetermined validated samples comprises samples clinically determined to have a neurological disease associated with elevated amyloid beta levels.

5. The method of claim 4, wherein the neurological disease is Alzheimer's Disease (AD), an amyloid plaque forming disease, or an AD like disease.

6. The method according to claim 1, wherein the amyloid beta levels are clinically determined by scanning analysis using an amyloid specific radiotracer.

7. The method of claim 6, wherein the radiotracer is PiB or F-18 AV-45.

8. The method according to claim 1, wherein the biomarkers are selected from any of those listed in TABLE 8.

9. The method according to claim 1, wherein two of the biomarkers are Amyloidβ42 and ApolipoproteinE or naturally occurring variants thereof.

10. The method of claim 9, wherein further biomarkers are BLC, cortisol, IgM, Pancreatic Polypeptide, VCAM1 and/or IL-17, or naturally occurring variants thereof.

11. The method according to claim 1, wherein the plurality of biomarkers forms a biomarker signature indicative of a theoretical neocortical amyloid loading.

12. The method according to claim 1, wherein the classification algorithm is also applied to clinical marker data obtained from the same plurality of predetermined validated samples determined in step a) or step b) in generating a set of relevant coefficients.

13. The method of claim 12, wherein the clinical marker data includes Clinical Dementia Rating or Body Mass Index, Age or CDR sum of boxes.

14. The method according to claim 1, wherein the classification algorithm undergoes cross-validation analysis.

15. The method according to claim 1, wherein the plurality of predetermined validated samples are obtained from a biological fluid.

16. The method of claim 15, wherein the biological fluid is blood, plasma, serum, urine, or cerebrospinal fluid.

17. A method for predicting the level of amyloid beta in a subject, comprising:
i) obtaining a set of relevant coefficients according to claim 1;
ii) obtaining a test biological sample from the subject;
iii) determining biomarker values for a plurality of biomarkers from the test biological sample, where the plurality of biomarkers corresponds to those determined in obtaining the set of relevant coefficients; and
iv) applying a classification algorithm to the biomarker values determined from step iii) and correlating this with the set of relevant coefficients to derive a theoretical amyloid beta level in the subject;
wherein the theoretical level amyloid beta predicts the risk of the subject developing a neurological disease.

18. The method of claim 17, wherein the set of relevant coefficients in step iv) is analysed by ROC or AUC.

19. The method of claim 17, wherein clinical marker data is obtained from the subject.

20. The method according to claim 17, wherein the test biological sample is a biological fluid.

21. The method of claim 20, wherein the biological fluid is blood, plasma, serum, urine, or cerebrospinal fluid.

22. A method for predicting a level of amyloid beta in a subject comprising:
a) employing a classification algorithm to a plurality of biomarker values and a plurality of amyloid beta level values obtained from a plurality of prevalidated samples to generate a set of relevant coefficients;
b) obtaining a test biological sample from the subject;
c) determining values in the test biological sample of b) for each of the biomarkers
d) applying the classification algorithm incorporating the set of relevant coefficients to the values determined from step c) to derive a theoretical amyloid beta level; and
e) determining a categorisation of a neurological disease state according to the output of step d),
wherein the theoretical level of beta amyloid predicts the risk of a subject developing a neurological disease.

23. The method of claim 22, wherein the classification algorithm is selected from the group consisting of Random Forest, Variable Importance Measures, Linear Discriminant Analysis (LDA), Diagonal Linear Discriminant Analysis (DLDA), Diagonal Quadratic Discriminant Analysis (DQDA), Support Vector Machines (SVM), Support Vector Regression (SVR) Neural Network, Analysis of Covariance (ANCOVA) and k-Nearest Neighbour method.

24. The method of claim 23, wherein the classification algorithm is Random Forest.

25. The method according to claim 1, wherein the plurality of predetermined validated samples comprises samples clinically determined to have a neurological disease associated with elevated amyloid levels.

26. The method according to claim 22, wherein the neurological disease is Alzheimer's Disease (AD), an amyloid plaque forming disease, or an AD like disease.

27. The method of claim 26, wherein the disease is Alzheimer's disease (AD).

28. The method according to claim 22, wherein the amyloid beta loading scores are clinically determined by scanning analysis using a radiotracer.

29. The method of claim 28, wherein the radiotracer is PiB or F-18 AV-45.

30. The method according to claim 22, wherein the biomarkers are selected from the biomarkers listed in TABLE 8.

31. The method according to claim 22, wherein two of the biomarkers are Amyloidβ42 and ApolipoproteinE or naturally occurring variants thereof.

32. The method of claim 31, wherein further biomarkers are BLC, cortisol, IgM, Pancreatic Polypeptide, VCAM1 and/or IL-17, or naturally occurring variants thereof.

33. The method according to claim 22, wherein the plurality of biomarkers forms a biomarker signature indicative of a theoretical neocortical amyloid loading.

34. The method according to claim 22, wherein the classification algorithm is also applied to clinical marker data obtained from the same plurality of predetermined validated samples determined in step a) in generating a set of relevant coefficients.

35. The method of claim 34, wherein the clinical marker data includes Clinical Dementia Rating or Body Mass Index, Age or CDR sum of boxes.

36. The method according to claim 22, wherein the clinical markers selected are from those listed in TABLE 2 or TABLE 3.

37. The method according to claim 22, wherein the classification algorithm undergoes cross-validation analysis.

38. The method according to claim 22, wherein the plurality of predetermined validated samples are obtained from a biological fluid.

39. The method of claim 38, wherein the biological fluid is blood, plasma, serum, urine, or cerebrospinal fluid.

40. The method according to claim 22, wherein the set of relevant coefficients in step d) are analysed by ROC or AUC.

41. A kit comprising the peptides, polypeptides, proteins, oligonucleotides or fragments thereof when used according to the method of claim 1, wherein the kit is used to determine the presence of biomarkers in a biological from a subject to determine whether the subject possesses or will develop a neurological disease.

42. A method of identifying drug or target compounds capable of assisting or treating a neurological disease by administering a drug or target compound to a patient and monitoring the change in the neocortical amyloid beta levels in a patient.

43. The method of claim 42, wherein the level of neocortical amyloid beta levels are monitored in a patient over a time period with the method according to claim 1.

44. The method of claim 42, wherein a drug or target compounds that causes a decrease of in the neocortical amyloid beta levels in a patient is indicative of a drug or target compound that may assist or treat a neurological disease associated with elevated amyloid beta levels in a patient.

45. The method according to claim 42, wherein the neurological disease is Alzheimer's Disease (AD), an amyloid plaque forming disease, or an AD like disease.

46. A computer system when used for predicting a level of amyloid beta in a subject, comprising:
i) inputting a set of relevant coefficients obtained according to claim 1;
ii) obtaining a test biological sample from the subject;
iii) determining values for a plurality of biomarkers from the test biological sample, where the plurality of biomarkers corresponds to those determined in obtaining the set of relevant coefficients; and
iv) applying a classification algorithm to the biomarker values determined from step iii) and correlating this with the set of relevant coefficients to derive a theoretical amyloid beta level in the subject;
wherein the theoretical amyloid beta level predicts the risk of the subject developing a neurological disease.

47. The computer system of claim 46, wherein the set of relevant coefficients in step iv) are analysed by ROC or AUC.

48. The computer system of claim 46, wherein clinical marker data is obtained from the subject.

49. The method according to claim 46, wherein the test biological sample is a biological fluid.

50. The method of claim 49, wherein the biological fluid is blood, plasma, serum, urine, or cerebrospinal fluid.

51. The method according to claim 46, wherein the biomarkers are selected from the biomarkers listed in TABLE 8.

52. The method according to claim 46, wherein two of the biomarkers are Amyloidβ42 and ApolipoproteinE or naturally occurring variants thereof.

53. The method of claim 52, wherein further biomarkers are BLC, cortisol, IgM, Pancreatic Polypeptide, VCAM1 and/or IL-17, or naturally occurring variants thereof:

54. A computer readable format comprising values and/or reference values obtained by the method of claim 1.

55. The method according to claim 34, wherein the clinical markers selected are from those listed in TABLE 2 or TABLE 3.

56. A kit comprising the peptides, polypeptides, proteins, oligonucleotides or fragments thereof when used according to the method of claim 22, wherein the kit is used to determine the presence of biomarkers in a biological from a subject to determine whether the subject possesses or will develop a neurological disease.

57. The method of claim 42, wherein the level of neocortical amyloid beta levels are monitored in a patient over a time period with the method according to claim 22.

58. A computer readable format comprising values and/or reference values obtained by the method of claim 22.
